(19) United States
(12) Patent Application Publication
(10) Pub. No.: US 2024/0060071 A1
(43) Pub. Date: Feb. 22, 2024

(54) TREATMENT OF CANCER ASSOCIATED WITH DYSREGULATED NOVEL OPEN READING FRAME PRODUCTS

(71) Applicants: Cambridge Enterprise Limited, Cambridge (GB); International Centre for Genetic Engineering and Biotechnology, Delhi (GB)

(72) Inventor: Sudhakaran PRABAKARAN, Acton, MA (US)

(21) Appl. No.: 18/267,327

(22) PCT Filed: Dec. 15, 2021
(86) PCT No.: PCT/IB2021/061801

§ 371 Date: Jun. 14, 2023

Related U.S. Application Data
(60) Provisional application No. 63/126,309, filed on Dec. 16, 2020.

(51) Int. Cl. C12N 15/113; C12Q 1/6886; C12Q 1/6809; C12N 15/86; C07K 16/32; A61K 48/00; A61P 35/00

(52) U.S. Cl. CPC C12N 15/1135; C12Q 1/6886; C12Q 1/6809; C12N 15/86; C07K 16/32; A61K 48/0041; A61P 35/00; C12Q 2600/158; C12N 2740/15043; C12N 2760/20222

(57) ABSTRACT

The present application features methods of treating a cancer associated with a dysregulated novel open reading frame (nORF) in which increased or reduced expression of the dysregulated nORF is associated with cancer.

### CROSS-REFERENCE TO RELATED APPLICATIONS

This application claims the benefit of U.S. Provisional Application No. 63/126,309 filed on Dec. 16, 2020, which is incorporated herein by reference in its entirety.

### BACKGROUND OF THE INVENTION

Many cancers are caused by genetic dysregulation of canonical genes known to be associated with the cancer. However, identifying how genetic dysregulation is linked to cancer pathology under these circumstances. Furthermore, providing an effective therapeutic remains a challenging endeavor. Accordingly, new methods of diagnosis and treatment are needed to better understand how these genetic dysregulations cause a wide range of cancers.

### SUMMARY OF THE INVENTION

In one aspect, the invention features a method of treating a cancer in a by identifying a sequence of a novel open reading frame (nORF) and a cancer associated therewith, wherein the sequence of the nORF is distinct from a canonical open reading frame (cORF) of a gene. The nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a noncancerous cell. The method further includes administering to the subject an inhibitor that reduces expression of the nORF to treat the cancer.

In another aspect, the invention features method of treating a cancer in a subject by administering to the subject an inhibitor that reduces expression of a nORF. The subject may have previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a noncancerous cell.

In some embodiments of either of the foregoing aspects, the method reduces expression of the nORF, e.g., by at least 5%, 10%, 15%, 20%, 25%, 30%, 40%, 50%, 60%, 70%, 80%, 90%, 95%, 97%, or 99%. The nORF may exhibit an increase (e.g. by at least 5%, 10%, 15%, 20%, 25%, 30%, 40%, 50%, 60%, 70%, 80%, 90%, 100%, 200%, 300%, 400%, 500%, or more) in expression, e.g., as compared to the nORF in normal (e.g., noncancerous) tissue.

In some embodiments of either of the above aspects, the inhibitor is a small molecule, a polynucleotide, or a polypeptide. The polynucleotide may include a miRNA, an antisense RNA, an shRNA, or an siRNA. The polypeptide may include an antibody or antigen-binding fragment thereof (e.g., an scFv).

In some embodiments, the inhibitor is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In another aspect, the invention features a method of treating a cancer in a subject by identifying a sequence of a nORF and a cancer associated therewith, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell. The method further includes administering to the subject an activator that increases expression of nORF to treat the cancer.

In another aspect, the invention features a method of treating a cancer in a subject by administering to the subject an activator that increases expression of a nORF. The subject may have previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell.

In some embodiments of either of the foregoing aspects, the method increases expression of the nORF, e.g., by at least 5%, 10%, 15%, 20%, 25%, 30%, 40%, 50%, 60%, 70%, 80%, 90%, 100%, 200%, 300%, 400%, 500%, or more. The nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the nORF in normal (e.g., noncancerous) tissue.

In some embodiments, the activator is a small molecule, a polynucleotide, or a polypeptide. The polynucleotide may include an antisense RNA. The polypeptide may include an antibody or antigen-binding fragment thereof (e.g., an scFv).

In some embodiments, the activator is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an AAV vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In another aspect, the invention features a method of treating a cancer in a subject by identifying a sequence of a nORF and a cancer associated therewith, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell. The method further includes providing a protein encoded by the nORF to the subject treat the cancer.

In another aspect, the invention features a method of treating a cancer in a subject by providing a protein encoded by a nORF to the subject. The subject may have previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell.

In some embodiments of either of the foregoing aspects, the method includes restoring the encoded protein product of the nORF. The method may include providing the protein product or a polynucleotide encoding the protein product. The method may include providing a vector (e.g., a viral vector) including the polynucleotide encoding the protein product.

In some embodiments, the viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In some embodiments of any of the above aspects, the encoded protein product of the nORF is less than about 100 amino acids.

In some embodiments, the method further includes performing a statistical analysis between the nORF and the cancer. The statistical analysis may measure a positive or negative association between the nORF and the cancer.

In some embodiments, the cancer is selected from the list consisting of breast invasive carcinoma, colon adenocarcinoma, esophageal carcinoma, head and neck squamous cell carcinoma, kidney chromophobe, kidney clear cell carcinoma, kidney papillary cell carcinoma, liver hepatocellular carcinoma, lung adenocarcinoma, lung squamous cell carcinoma, prostrate adenocarcinoma, stomach adenocarcinoma, thyroid carcinoma, and uterine corpus endometrioid carcinoma

In some embodiments, the nORF is selected from Table 1.

In some embodiments, the nORF is selected from Table 2.

In some embodiments, the nORF is selected from Table 3.

In some embodiments, the nORF is selected from Table 4.

In some embodiments, the nORF is selected from Table 5.

In some embodiments, the nORF is not HOXB-AS3.

In some embodiments, the cancer is not colorectal cancer.

In some embodiments, the nORF is not PINT87aa (LING-PINT).

In some embodiments, the cancer is not glioblastoma.

### Definitions

As used herein, a “novel open reading frame” or “nORF” refers to an open reading frame that is transcribed in a cell and consists of a sequence that is distinct from a canonical open reading frame (cORF) transcribed from a gene. The nORF may be present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene. The nORF may be any unannotated genetic sequence that is transcribed in a cell.

As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene comprising the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

### BRIEF DESCRIPTION OF THE DRAWINGS

FIG. 1 is a schematic representation of nORFs and their genomic locations. nORFs (yellow boxes) include short ORFs (sORFs) which are ORFs less than 100 aa, alternative ORFs (altORFs) present in alternative frames of canonical ORFs within protein-coding genes and undefined ORFs which have as of yet not been identified by other studies. These nORFs can be found both within protein-coding (including 5′UTR, 3′UTR, CDS or overlapping CDS and the UTRs) and noncoding regions. They can also be present antisense to genes. ORFs identified within Pseudogenes and Denovogenes are also included under the categorization of nORFs. Reg.: Regulatory regions

FIGS. 2A-2E are graphs showing differentially expressed nORF transcripts in cancer. FIG. 2A shows total number of differentially expressed nORF transcripts by cancer type compared with NAT. FIG. 2B shows total number of differentially expressed nORF transcripts by cancer type compared with GTEx FIG. 2C shows nORF transcripts uniquely up- or down-regulated in a single cancer type compared with NAT. FIG. 2D shows nORF transcripts uniquely up- or down-regulated in a single cancer type compared with GTEx normal tissue. FIG. 2E shows reproducibility of differential expression results using normal adjacent tissue and GTEx normal tissue. nORF transcripts identified as differentially expressed when comparing cancer tissue with normal adjacent tissue, showing the proportion of nORF transcripts also differentially expressed when comparing cancer tissue with GTEx tissue (left panel: up-regulated nORF transcripts, right panel: down-regulated nORF transcripts)

FIGS. 3A and 3B are graphs showing survival analysis of nORF transcripts. FIG. 3A shows association of nORF transcript expression with overall patient survival. Number of differentially expressed nORF transcripts significantly associated with survival at different adjusted p value thresholds, by cancer type. FIG. 3B shows Kaplan Meier curves showing overall patient survival in high and low expression groups for reproducibly differentially expressed nORF transcripts. Showing Kaplan Meier curves, nORF transcript ID and further transcript details for the four nORF transcripts most significantly associated with prognosis, in Kidney Clear Cell Carcinoma. The cohort was divided into high and low nORF transcript expression groups using the Maximally Selected Rank Statistic, and Kaplan Meier survival curves were generated with a 95% confidence interval. Survival probabilities were compared using the log-rank test and p values adjusted for multiple testing. Overall survival times were fitted to a Cox proportional hazards regression model and hazard ratio calculated from the fitted coefficients.

FIG. 4 is a schematic diagram showing the scope of the anlaysis. We obtained RNA-Seq transcript-level expected counts for samples in TCGA and GTEx, match normal and cancer tissues, identify expressed nORF transcripts and perform differential expression and survival analysis.

FIG. 5 is a schematic drawing identifying expressed transcripts encoding novel open reading frames. Computational pipeline used to identify transcripts containing novel open reading frames 1, and the types of mapping between nORF and transcript genomic coordinates accepted and rejected in this pipeline.

FIGS. 6A-6E are graphs identifying expressed transcripts encoding novel open reading frames. Frequency of canonical transcript Ensembl biotypes for noncoding transcripts containing nORFs, for all nORF transcripts (FIG. 6A) and expressed nORF transcripts (FIG. 6B) considered in this study. FIG. 6C shows a rainfall graph showing the genomic distribution of expressed nORF transcripts, measured in nucleotides from the nORF start site, with a pseudo-count of 0.0001. FIG. 6D shows frequency of expressed nORF transcripts by chromosome and strand. FIG. 6E shows distribution of ORF length for novel and canonical ORFs, by chromosome.

FIG. 7 is a graph showing expression of nORF transcripts in normal tissues. Mean CPM value (TMM normalized) for nORF transcripts by tissue, log transformed with a pseudo-count of 0.0001. Mean expression of nORF transcripts compared with protein coding, long intergenic non-coding and antisense transcripts across GTEx normal tissues.

FIGS. 8A and 8B are graphs showing transcript expression across GTEx tissues. Means and standard deviations for TMM normalized expression counts (CPM) are calculated tissue-wise across all tissues included from the GTEx dataset and a median coefficient of variation (CV) is calculated from tissue-wise variations. Transcripts are classified as canonical protein coding, non-coding or novel based on the workflow presented in FIGS. 5 and 6A and as detailed in Materials and Methods. FIG. 8A shows tissue-wise mean and standard deviation for lung tissue—a random sample of 1000 transcripts from each class is shown to limit overplotting. FIG. 8B shows CV distributions for each transcript class are compared using a non-parametric Wilcoxon statistical test, and p-values are displayed. Transcript subsets for ‘non-coding’ and ‘novel’ transcripts are produced by stratifying by transcript type, and CV comparisons for antisense and lincRNA transcripts are performed in isolation.

FIGS. 9A-9D are graphs showing frequently expressed nORF transcripts across cancer and normal reference samples. Percentage of samples exhibiting transcript expression greater than 0.5 CPM for each expressed nORF transcript. Representative plot shown for breast invasive carcinoma tissue compared with normal adjacent tissue (FIG. 9A) and GTEx normal tissue (FIG. 9B). nORF transcripts identified as frequently expressed are annotated in FIGS. 9C and 9D. Most frequent profiles of frequently expressed nORF transcripts across cancer types, considering cancer and normal adjacent tissue (FIG. 9C) and cancer and GTEx normal tissue (FIG. 9D).

FIGS. 10A-10G are graphs showing differentially expressed nORF transcripts in cancer, corresponding analysis using a fold change threshold of 1.5, with associated survival analysis. FIG. 10A shows total number of differentially expressed nORF transcripts by cancer type compared with NAT. FIG. 10B shows total number of differentially expressed nORF transcripts by cancer type compared with GTEx. FIG. 10C shows nORF transcripts uniquely up- or down-regulated in a single cancer type compared with NAT. FIG. 10D shows nORF transcripts uniquely up- or down-regulated in a single cancer type compared with GTEx normal tissue. FIG. 10E shows reproducibility of differential expression results using normal adjacent tissue and GTEx normal tissue. nORF transcripts identified as differentially expressed when comparing cancer tissue with normal adjacent tissue, showing the proportion of nORF transcripts also differentially expressed when comparing cancer tissue with GTEx tissue (upper: up-regulated nORF transcripts, lower: down-regulated nORF transcripts). FIG. 10F shows association of nORF transcript expression with overall patient survival. Number of differentially expressed nORF transcripts significantly associated with survival at different adjusted p value thresholds, by cancer type. FIG. 10G shows Kaplan Meier curves showing overall patient survival in high and low expression groups for reproducibly differentially expressed nORF transcripts. Showing Kaplan Meier curves, nORF transcript ID and further transcript details for four nORF transcripts uniquely and reproducibly up-expressed in a single disease, and where high expression is associated with poor prognosis. The cohort was divided into high and low nORF transcript expression groups using the Maximally Selected Rank Statistic, and Kaplan Meier survival curves were generated with a 95% confidence interval. Survival probabilities were compared using the log-rank test and p values adjusted for multiple testing. Overall survival times were fitted to a Cox proportional hazards regression model and hazard ratio calculated from the fitted coefficients.

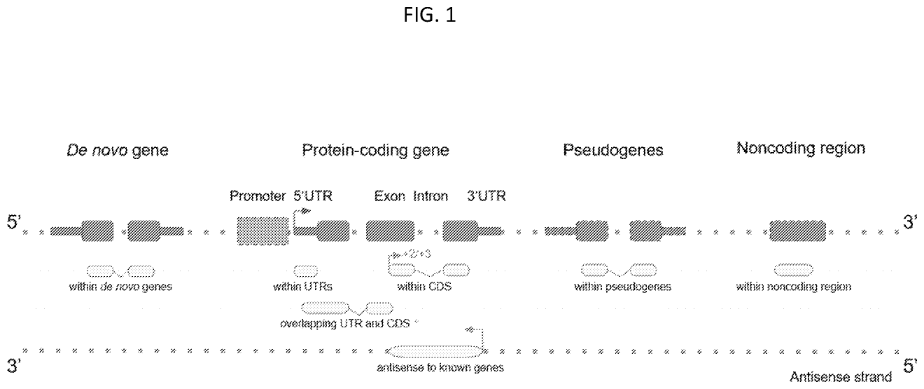

### DETAILED DESCRIPTION

Described herein are methods of diagnosing and treating a cancer associated with dysregulated novel open reading frames (nORFs). Many cancers are caused by dysregulation (e.g., upregulation or downregulation) in a gene or a genetic variant that is associated with the cancer. However, it was previously unclear how certain cancers are caused in which no dysregulation of a canonical gene or a canonical open reading frame (cORF) associated with the gene is present and no variant is known. The present invention is premised, in part, upon the discovery of dysregulation of certain novel open reading frames (nORFs) that are distinct from canonical open reading frames (cORF) of genes. In these instances, the dysregulation (e.g., upregulation or downregulation) imparts a deleterious effect on the nORF, in some instances, with or without substantially impacting a protein encoded by a cORF. In particular, the present invention features methods of treating cancer associated with a dysregulated nORF in which differential expression (e.g., increased or decreased expression) of the nORF is observed. With increased or decreased expression, the gene product encoded by the dysregulated nORF is increased or decreased as compared to the nORF, e.g., in a noncancerous cell. The methods of diagnosis and treatment are described in more detail below.

### Methods of Diagnosis

Genetic testing offers one avenue by which a patient may be diagnosed as having or is at risk of developing a particular cancer. For example, a genetic analysis can be used to determine whether a patient has a nORF associated with a cancer. The nORF may be present in any region of a gene, such as within the cORF, a 5′ untranslated region (UTR) of the cORF, a 3′ UTR of the cORF, an intronic region of the cORF, or an intergenic region of the cORF, The nORF may be present within an overlapping region of the cORF in an alternate reading frame, a 5′ UTR of the cORF, a 3′ UTR of the cORF, an intronic region of the cORF, or an intergenic region of the cORF. The nORF may be present in a region that is not associated with the cORF of the gene.

Exemplary genetic tests that can be used to determine whether a patient has such nORF include polymerase chain reaction (PCR) methods known in the art, such as DNA and RNA sequencing. nORF sequences may be identified de novo, e.g., using computational or statistical methods. Furthermore, nORF sequences may be identified from publicly available databases in genomic sequences in which the nORF was not previously identified and/or annotated as a sequence that was transcribed, and/or translated.

nORF sequences may be identified as being linked to a particular cancer by using a statistical analysis between the dysregulated nORF and the cancer. The statistical analysis may measure a positive or negative association between the dysregulated nORF and the cancer (see, e.g., Example 1).

To examine the functional importance of a nORF separately from a canonical coding sequence, datasets, such as the Genome Aggregation Database, may be used.

### Methods of Treatment

The invention features methods of treating a subject having a dysregulated nORF that has differential expression (e.g., increased or decreased expression). The dysregulated nORF may exhibit an increase (e.g., by at least 5%, 10%, 15%, 20%, 25%, 30%, 40%, 50%, 60%, 70%, 80%, 90%, 100%, 200%, 300%, 400%, 500%, or more) in expression, e.g., as compared to the nORF in normal (e.g., noncancerous) tissue. The dysregulated nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the dysregulated nORF in normal (e.g., noncancerous) tissue. The subject may be first determined to have the dysregulated nORF and then may subsequently be treated for the cancer. The subject may have previously been determined to have the dysregulated nORF and is then treated for the cancer. The treatment varies according to the dysregulated nORF associated with the cancer. For example, the treatment may include an inhibitor that targets the dysregulated nORF to decrease (e.g., by at least 5%, 10%, 15%, 20%, 25%, 30%, 40%, 50%, 60%, 70%, 80%, 90%, 95%, 97%, or 99%) expression of an upregulated nORF. The treatment may include an activator that targets the dysregulated nORF to increase (e.g., by at least 5%, 10%, 15%, 20%, 25%, 30%, 40%, 50%, 60%, 70%, 80%, 90%, 100%, 200%, 300%, 400%, 500%, or more) expression of a downregulated nORF. Alternatively, or in addition, the treatment may include providing the nORF or a protein encoded by the nORF to restore levels of the nORF.

### Inhibitors

The methods of treatment and diagnosis described herein may include providing an inhibitor that targets the dysregulated nORF. The inhibitor may reduce (e.g., by at least 5%, 10%, 15%, 20%, 25%, 30%, 40%, 50%, 60%, 70%, 80%, 90%, 95%, 97%, or 99%) an amount or activity of the dysregulated nORF, such as to prevent the deleterious effect of the dysregulated nORF. The inhibitor may target the polynucleotide containing the nORF or the protein encoded by the nORF. The inhibitor may be a small molecule, a polynucleotide, or a polypeptide. Suitable small molecules may be determined or identified by using computational analysis based on the structure of the dysregulated nORF as determined by a protein folding algorithm. The small molecule may target any region of the dysregulated nORF. The small molecule may target the nORF or the protein encoded by the nORF. Suitable polypeptides for reducing an activity or amount of the dysregulated nORF include, for example, an antibody or antigen-binding fragment thereof that binds to the dysregulated nORF (e.g., a single chain antibody or antigen-binding fragment thereof). Suitable polynucleotides that can reduce an amount or activity of the dysregulated nORF include RNA. For example, an RNA for reducing an activity or amount of the dysregulated nORF may be, for example, a miRNA, an antisense RNA, an shRNA, or an siRNA. The miRNA, antisense RNA, shRNA, or siRNA may target a region of RNA (e.g., dysregulated nORF gene) to reduce expression of the dysregulated nORF. The polynucleotide may be an aptamer, e.g., an RNA aptamer that binds to and/or reduces an amount and/or activity of the dysregulated nORF or the protein encoded by the dysregulated nORF. The inhibitor may be provided directly or may be provided by a vector (e.g., a viral vector) encoding the inhibitor. The inhibitor may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

Nucleic Acid Mediated Knockdown

Using the compositions and methods described herein, a patient with a cancer may be administered an interfering RNA molecule, a composition containing the same, or a vector encoding the same, so as to reduce or suppress the expression of a dysregulated nORF. Exemplary interfering RNA molecules that may be used in conjunction with the compositions and methods described herein are siRNA molecules, miRNA molecules, shRNA molecules, and antisense RNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the dysregulated nORF gene with complementary nucleotide sequences by degrading mRNA (via the Dicer and RISC pathways) after transcription, thereby preventing translation. miRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly(A) tail. shRNA is an artificial RNA molecule with a tight hairpin turn that can be used to silence target gene expression via RNA interference. Antisense RNA are also short single stranded molecules that hybridize to a target RNA and prevent translation by occluding the translation machinery, thereby reducing expression of the target (e.g., the dysregulated nORF).

Antibody Mediated Knockdown

Using the compositions and methods described herein, a patient with a cancer may be provided an antibody or antigen-binding fragment thereof, a composition containing the same, a vector encoding the same, or a composition of cells containing a vector encoding the same, so as to suppress or reduce the activity of the dysregulated nORF. In some embodiments of the compositions and methods described herein, an antibody or antigen-biding fragment thereof may be used that binds to and reduces or eliminates the activity of the dysregulated nORF. The antibody may be monoclonal or polyclonal. In some embodiments, the antigen-binding fragment is an antibody that lacks the Fc portion, an F(ab′)2, a Fab, an Fv, or an scFv. The antigen-binding fragment may be an scFv.

One of ordinary skill in the art will appreciate that an antibody may include four polypeptides: two identical copies of a heavy chain polypeptide and two copies of a light chain polypeptide. Each of the heavy chains contains one N-terminal variable (V_{H}) region and three C-terminal constant (CH1, CH2 and CH3) regions, and each light chain contains one N-terminal variable (VL) region and one C-terminal constant (C_{L}) region. Thus, one of skill in the art would appreciate that as described herein, a vector that includes a transgene that encodes a polypeptide that is an antibody may be a single transgene that encodes a plurality of polypeptides. Also contemplated is a vector that includes a plurality of transgenes, each transgene encoding a separate polypeptide of the antibody. All variations are contemplated herein. The variable regions of each pair of light and heavy chains form the antigen binding site of an antibody. The transgene which encodes an antibody directed against the dysregulated nORF can include one or more transgene sequences, each of which encodes one or more of the heavy and/or light chain polypeptides of an antibody. In this respect, the transgene sequence which encodes an antibody directed against the dysregulated nORF can include a single transgene sequence that encodes the two heavy chain polypeptides and the two light chain polypeptides of an antibody. Alternatively, the transgene sequence which encodes an antibody directed against the dysregulated nORF can include a first transgene sequence that encodes both heavy chain polypeptides of an antibody, and a second transgene sequence that encodes both light chain polypeptides of an antibody. In yet another embodiment, the transgene sequence which encodes an antibody can include a first transgene sequence encoding a first heavy chain polypeptide of an antibody, a second transgene sequence encoding a second heavy chain polypeptide of an antibody, a third transgene sequence encoding a first light chain polypeptide of an antibody, and a fourth transgene sequence encoding a second light chain polypeptide of an antibody.

In some embodiments, the transgene that encodes the antibody includes a single open reading frame encoding a heavy chain and a light chain, and each chain is separated by a protease cleavage site.

In some embodiments, the transgene encodes a single open reading frame encoding both heavy chains and both light chains, and each chain is separate by protease cleavage site.

In some embodiments, full-length antibody expression can be achieved from a single transgene cassette using 2A peptides, such as foot-and-mouth disease virus (FMDV) equine rhinitis A, porcine teschovirus-1, and Thosea asigna virus 2A peptides, which are used to link two or more genes and allow the translated polypeptide to be self-cleaved into individual polypeptide chains (e.g., heavy chain and light chain, or two heavy chains and two light chains). Thus, in some embodiments, the transgene encodes a 2A peptide in between the heavy and light chains, optionally with a flexible linker flanking the 2A peptide (e.g., GSG linker). The transgene may further include one or more engineered cleavage sequences, e.g., a furin cleavage sequence to remove the 2A peptide residues attached to the heavy chain or light chain. Exemplary 2A peptides are described, e.g., in Chng et al MAbs 7: 403-412, 201f5, and Lin et al. Front. Plant Sci 9:1379, 2018, the disclosures of which are hereby incorporated by reference in their entirety.

In some embodiments, the antibody is a single-chain antibody or antigen-binding fragment thereof expressed from a single transgene.

### Activators

The methods of treatment and diagnosis described herein may include providing an activator that targets the dysregulated nORF. The activator may increase (e.g., by at least 5%, 10%, 15%, 20%, 25%, 30%, 40%, 50%, 60%, 70%, 80%, 90%, 100%, 200%, 300%, 400%, 500%, or more) an amount or activity of the dysregulated nORF, such as to prevent the deleterious effect of the dysregulated nORF. The activator may target the polynucleotide containing the nORF or the protein encoded by the nORF. The activator may be a small molecule, a polynucleotide, or a polypeptide. Suitable small molecules may be determined or identified by using computational analysis based on the structure of the dysregulated nORF as determined by a protein folding algorithm. The small molecule may target any region of the dysregulated nORF. The small molecule may target the nORF or the protein encoded by the nORF. Suitable polypeptides for increasing an activity or amount of the dysregulated nORF include, for example, an antibody or antigen-binding fragment thereof that binds to the dysregulated nORF (e.g., a single chain antibody or antigen-binding fragment thereof). Suitable polynucleotides that can increase an amount or activity of the dysregulated nORF include RNA. For example, an RNA for increasing an activity or amount of the dysregulated nORF may be, for example, an antisense RNA. The antisense RNA may target a region of RNA (e.g., dysregulated nORF gene) upstream of the primary nORF open reading frame to reduce expression of the upstream nORFs, thereby dedicating the translation machinery to the primary nORF in order to increase expression of the primary nORF. The polynucleotide may be an aptamer, e.g., an RNA aptamer that binds to and/or increases an amount and/or activity of the dysregulated nORF or the protein encoded by the dysregulated nORF. The activator may be provided directly or may be provided by a vector (e.g., a viral vector) encoding the activator. The activator may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

nORF Replacement

The present invention also features methods of treating a cancer by administering or providing a nORF or a protein encoded by the nORF. The therapy may restore the encoded protein product of the nORF, such as to replace the nORF that is no longer present due to downregulation. The therapy may include providing the protein product or a polynucleotide encoding the protein product. The method may include providing a vector (e.g., a viral vector) that encodes the protein product. Alternatively, the protein encoded by the nORF may be administered directly, e.g., as an enzyme replacement therapy. The nORF or a polynucleotide encoding the nORF (e.g., a vector, e.g., a viral vector) may be formulated, e.g., in a pharmaceutical composition containing a pharmaceutically acceptable carrier. The composition can be administered by any suitable method known in the art to the skilled artisan. The composition may be formulated in a virus or a virus-like particle.

In some embodiments, the length of the nORF is less than about 100 amino acids (e.g., from about 50 to 100, 50 to 90, 50 to 80, 60 to 90, 60 to 80, 70 to 100, 70 to 90, 70 to 80, 80 to 100, or 90 to 100 amino acids).

### Viral Vectors for Expression

Viral genomes provide a rich source of vectors that can be used for the efficient delivery of exogenous genes into a mammalian cell. The gene to be delivered may include an activator or inhibitor that targets a dysregulated nORF, such as an RNA (e.g., an aptamer, a miRNA, an antisense RNA, an shRNA, or an siRNA). Alternatively, the gene to be delivered may include the nORF for replacement. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

Retro viral Vectors

The delivery vector used in the methods described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klunnp et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be a ubiquitous promoter. Alternatively, the promoter may be a tissue specific promoter, such as a myeloid cell-specific or hepatocyte-specific promoter. Suitable promoters that may be used with the compositions described herein include CD11 b promoter, sp146/p47 promoter, CD68 promoter, sp146/gp9 promoter, elongation factor 1 α (EF1α) promoter, EF1α short form (EFS) promoter, phosphoglycerate kinase (PGK) promoter, α-globin promoter, and β-globin promoter. Other promoters that may be used include, e.g., DC172 promoter, human serum albumin promoter, alpha1 antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region ((3LCR).

Methods of Measuring nORF Gene Expression

Preferably, the compositions and methods of the disclosure are used to facilitate expression of a nORF at physiologically normal levels in a patient (e.g., a human patient), decrease expression of an upregulated nORF, or increase expression of a downregulated nORF. The therapeutic agents of the disclosure, for example, may reduce the dysregulated nORF expression in a human subject. For example, the therapeutic agents of the disclosure may reduce dysregulated nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%. Alternatively, the therapeutic agents of the disclosure may increase the dysregulated nORF expression in a human subject. For example, the therapeutic agents of the disclosure may increase dysregulated nORF expression, e.g., by at least 5%, 10%, 15%, 20%, 25%, 30%, 40%, 50%, 60%, 70%, 80%, 90%, 100%, 200%, 300%, 400%, 500%, or more.

The expression level of the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the dysregulated nORF. Protein concentrations can also be assessed using functional assays, such as MDP detection assays. Expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the nORF) detection that may be used in conjunction with the compositions and methods described herein include imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., the nORF in a noncancerous cell or the dysregulated nORF). Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Cancer

A number of cancers are known in the art that are contemplated in conjunction with the methods described herein. The present invention contemplates treatment of a cancer in which a nORF exhibits increased or decreased expression, e.g., relative to a noncancerous cell.

The method may reduce the size (e.g., by 5%, 10%, 15%, 20%, 25%, 30%, 40%, 50%, 60%, 70%, 80%, 90%, 95%, 97%, or 99%) of a tumor (e.g., a breast tumor). The method may decrease or slow (e.g., by at least 5%, 10%, 15%, 20%, 25%, 30%, 40%, 50%, 60%, 70%, 80%, 90%, 95%, 97%, or 99%) the progression of cancer. The method may decrease (e.g., by at least 5%, 10%, 15%, 20%, 25%, 30%, 40%, 50%, 60%, 70%, 80%, 90%, 95%, 97%, or 99%) the risk of developing cancer. The method may decrease (e.g., by at least 5%, 10%, 15%, 20%, 25%, 30%, 40%, 50%, 60%, 70%, 80%, 90%, 95%, 97%, or 99%) the risk of developing cancer.

In some embodiments, the cancer is selected from the list consisting of breast invasive carcinoma, colon adenocarcinoma, esophageal carcinoma, head and neck squamous cell carcinoma, kidney chromophobe, kidney clear cell carcinoma, kidney papillary cell carcinoma, liver hepatocellular carcinoma, lung adenocarcinoma, lung squamous cell carcinoma, prostrate adenocarcinoma, stomach adenocarcinoma, thyroid carcinoma, and uterine corpus endometrioid carcinoma.

In some embodiments, the nORF is selected from Table 1.

In some embodiments, the nORF is selected from Table 2.

In some embodiments, the nORF is selected from Table 3.

In some embodiments, the nORF is selected from Table 4.

In some embodiments, the nORF is selected from Table 5.

TABLE 1

| | | | | | | | | | prop_ex- | | | |
|---|---|---|---|---|---|---|---|---|---|---|---|---|
| | | | tags.un- | | | | decide- | | pressed_dis- | prop_ex- | is.freq.ex- | |
| transcript | disease | tags.logFC | shrunk.logFC | tags.logCPM | tags.PValue | tags.FDR | test | tissue | ease | pressed_tissue | pressed | n |
| ENST00000523301.1 | Breast.Invasive.Carcinoma | −3.30991 | −3.32261 | −0.58509 | 2.21E−144 | 2.47E−142 | Down | Breast | 0.124542 | 0.932584 | TRUE | 1 |
| ENST00000437764.5 | Colon.Adenocarcinoma | −3.19909 | −3.20441 | 0.71864 | 3.57E−83 | 1.54E−81 | Down | Colon | 0.491289 | 0.830619 | TRUE | 1 |
| ENST00000546949.5 | Colon.Adenocarcinoma | −2.26234 | −2.27135 | 0.821437 | 2.10E−56 | 4.37E−55 | Down | Colon | 0.209059 | 0.970684 | TRUE | 1 |
| ENST00000606723.2 | Colon.Adenocarcinoma | −1.51514 | −1.51623 | 2.316787 | 2.43E−16 | 1.30E−15 | Down | Colon | 1 | 0.986971 | TRUE | 1 |
| ENST00000559012.1 | Colon.Adenocarcinoma | −1.59794 | −1.60438 | 0.562682 | 8.05E−16 | 4.20E−15 | Down | Colon | 0.243902 | 0.889251 | TRUE | 1 |
| ENST00000423477.2 | Colon.Adenocarcinoma | −1.53882 | −1.54262 | 1.309002 | 4.35E−11 | 1.81E−10 | Down | Colon | 0.407666 | 0.856678 | TRUE | 1 |
| ENST00000510937.1 | Colon.Adenocarcinoma | −1.55399 | −1.55826 | 2.153752 | 1.42E−04 | 3.88E−04 | Down | Colon | 0.209059 | 0.736156 | TRUE | 1 |
| ENST00000503525.2 | Glioblastoma.Multiforme | −4.36742 | −4.37867 | 0.359183 | 2.04E−30 | 4.88E−29 | Down | Brain | 0.196078 | 0.882404 | TRUE | 1 |
| ENST00000564460.2 | Glioblastoma.Multiforme | −2.28238 | −2.29681 | −1.49028 | 1.11E−26 | 2.26E−25 | Down | Brain | 0.071895 | 0.737805 | TRUE | 1 |
| ENST00000527986.5 | Glioblastoma.Multiforme | −1.33039 | −1.33063 | 4.07868 | 1.80E−11 | 1.48E−10 | Down | Brain | 1 | 0.994774 | TRUE | 1 |
| ENST00000517833.1 | Glioblastoma.Multiforme | −1.78958 | −1.79505 | −0.72001 | 1.14E−07 | 6.87E−07 | Down | Brain | 0.339869 | 0.847561 | TRUE | 1 |
| ENST00000425474.5 | Liver.Hepatocellular.Carcinoma | −3.54054 | −3.54576 | −1.6575 | 1.26E−48 | 9.95E−47 | Down | Liver | 0.441734 | 0.981818 | TRUE | 1 |
| ENST00000530595.1 | Liver.Hepatocellular.Carcinoma | −2.69137 | −2.71285 | −0.80043 | 2.98E−37 | 1.23E−35 | Down | Liver | 0.073171 | 0.809091 | TRUE | 1 |
| ENST00000446875.1 | Liver.Hepatocellular.Carcinoma | −2.00581 | −2.02304 | −1.68454 | 3.77E−20 | 5.47E−19 | Down | Liver | 0.03252 | 0.727273 | TRUE | 1 |
| ENST00000606083.1 | Liver.Hepatocellular.Carcinoma | −2.48178 | −2.4869 | −2.69207 | 8.34E−20 | 1.18E−18 | Down | Liver | 0.390244 | 0.909091 | TRUE | 1 |
| ENST00000599831.6 | Lung.Squamous.Cell.Carcinoma | −3.33282 | −3.34567 | −0.8325 | 7.73E−50 | 8.12E−49 | Down | Lung | 0.116466 | 0.826389 | TRUE | 1 |
| ENST00000593298.5 | Lung.Squamous.Cell.Carcinoma | −2.36761 | −2.36909 | 1.857262 | 3.17E−20 | 1.48E−19 | Down | Lung | 0.544177 | 0.979167 | TRUE | 1 |
| ENST00000500365.2 | Lung.Squamous.Cell.Carcinoma | −1.72548 | −1.73188 | −0.78217 | 1.99E−14 | 7.57E−14 | Down | Lung | 0.285141 | 0.954861 | TRUE | 1 |
| ENST00000562172.2 | Ovarian.Serous.Cystadenocarcinoma | −3.73651 | −3.7393 | −0.07435 | 7.84E−64 | 2.69E−62 | Down | Ovary | 0.768496 | 0.988636 | TRUE | 1 |
| ENST00000603191.2 | Ovarian.Serous.Cystadenocarcinoma | −2.27985 | −2.28878 | −0.66083 | 1.02E−33 | 1.14E−32 | Down | Ovary | 0.162291 | 0.965909 | TRUE | 1 |
| ENST00000544983.1 | Ovarian.Serous.Cystadenocarcinoma | −2.68333 | −2.70084 | −1.31479 | 7.91E−16 | 3.84E−15 | Down | Ovary | 0.083532 | 0.920455 | TRUE | 1 |
| ENST00000609585.1 | Ovarian.Serous.Cystadenocarcinoma | −2.23904 | −2.25938 | −2.55654 | 2.82E−10 | 1.00E−09 | Down | Ovary | 0.057279 | 0.727273 | TRUE | 1 |
| ENST00000624128.1 | Ovarian.Serous.Cystadenocarcinoma | −1.7461 | −1.7567 | −0.94322 | 3.12E−07 | 9.02E−07 | Down | Ovary | 0.133652 | 0.829545 | TRUE | 1 |
| ENST00000599889.1 | Ovarian.Serous.Cystadenocarcinoma | −1.52001 | −1.5301 | −1.92213 | 1.37E−06 | 3.79E−06 | Down | Ovary | 0.083532 | 0.829545 | TRUE | 1 |
| ENST00000607074.1 | Pancreatic.Adenocarcinoma | −4.59636 | −4.60378 | −1.62461 | 5.36E−89 | 1.01E−86 | Down | Pancreas | 0.230337 | 0.976048 | TRUE | 1 |
| ENST00000434233.1 | Pancreatic.Adenocarcinoma | −4.25976 | −4.26292 | −1.04319 | 9.48E−30 | 1.64E−28 | Down | Pancreas | 0.303371 | 0.982036 | TRUE | 1 |
| ENST00000623374.1 | Pancreatic.Adenocarcinoma | −3.94505 | −3.95137 | 0.172245 | 4.24E−27 | 6.39E−26 | Down | Pancreas | 0.140449 | 0.754491 | TRUE | 1 |
| ENST00000416769.1 | Pancreatic.Adenocarcinoma | −1.69818 | −1.6985 | 2.941975 | 2.03E−24 | 2.67E−23 | Down | Pancreas | 1 | 0.988024 | TRUE | 1 |
| ENST00000316124.3 | Pancreatic.Adenocarcinoma | −2.16342 | −2.17482 | −0.27753 | 3.22E−15 | 2.52E−14 | Down | Pancreas | 0.106742 | 0.916168 | TRUE | 1 |
| ENST00000602367.1 | Pancreatic.Adenocarcinoma | −1.73944 | −1.74017 | 2.677233 | 2.25E−11 | 1.36E−10 | Down | Pancreas | 1 | 0.988024 | TRUE | 1 |
| ENST00000441095.2 | Pancreatic.Adenocarcinoma | −1.94004 | −1.94654 | −0.34137 | 2.30E−07 | 1.00E−06 | Down | Pancreas | 0.140449 | 0.886228 | TRUE | 1 |
| ENST00000598755.1 | Pancreatic.Adenocarcinoma | −1.46614 | −1.4731 | −1.16106 | 5.53E−05 | 1.89E−04 | Down | Pancreas | 0.179775 | 0.874251 | TRUE | 1 |
| ENST00000510714.1 | Prostate.Adenocarcinoma | −2.47935 | −2.50661 | −1.67853 | 3.99E−38 | 1.39E−36 | Down | Prostate | 0.008097 | 0.78 | TRUE | 1 |
| ENST00000590398.5 | Prostate.Adenocarcinoma | −1.73127 | −1.73401 | −0.28322 | 6.99E−05 | 2.29E−04 | Down | Prostate | 0.423077 | 0.74 | TRUE | 1 |
| ENST00000324348.7 | Skin.Cutaneous.Melanoma | −3.33778 | −3.33826 | 3.013132 | 4.64E−153 | 5.77E−151 | Down | Skin | 1 | 1 | TRUE | 1 |
| ENST00000593151.2 | Skin.Cutaneous.Melanoma | −3.83717 | −3.86006 | −1.7356 | 2.59E−34 | 2.83E−33 | Down | Skin | 0.088235 | 0.976577 | TRUE | 1 |
| ENST00000440578.1 | Skin.Cutaneous.Melanoma | −4.06572 | −4.07313 | −0.77426 | 9.39E−24 | 6.88E−23 | Down | Skin | 0.254902 | 0.983784 | TRUE | 1 |
| ENST00000438290.2 | Skin.Cutaneous.Melanoma | −3.54853 | −3.55433 | 1.540235 | 8.84E−22 | 5.97E−21 | Down | Skin | 0.205882 | 0.992793 | TRUE | 1 |
| ENST00000534398.1 | Skin.Cutaneous.Melanoma | −2.72236 | −2.73633 | 0.197512 | 4.67E−18 | 2.66E−17 | Down | Skin | 0.088235 | 0.897297 | TRUE | 1 |
| ENST00000592556.5 | Skin.Cutaneous.Melanoma | −3.46282 | −3.47829 | −1.28403 | 1.09E−16 | 5.82E−16 | Down | Skin | 0.137255 | 0.906306 | TRUE | 1 |
| ENST00000592918.5 | Skin.Cutaneous.Melanoma | −2.19119 | 2.19311 | 2.396674 | 1.77E−09 | 6.38E−09 | Down | Skin | 0.333333 | 0.994595 | TRUE | 1 |
| ENST00000555438.2 | Skin.Cutaneous.Melanoma | −2.24489 | −2.25699 | −0.14271 | 6.71E−07 | 2.03E−06 | Down | Skin | 0.196078 | 0.704505 | TRUE | 1 |
| ENST00000579458.1 | Stomach.Adenocarcinoma | −3.15279 | −3.16564 | 0.789008 | 5.31E−34 | 5.78E−33 | Down | Stomach | 0.130751 | 0.729885 | TRUE | 1 |
| ENST00000413825.6 | Testicular.Germ.Cell.Tumor | −9.00634 | −9.86543 | −3.44749 | 0 | 0 | Down | Testis | 0 | 0.890909 | TRUE | 1 |
| ENST00000593954.5 | Testicular.Germ.Cell.Tumor | −8.85845 | −9.50119 | −3.35077 | 0 | 0 | Down | Testis | 0.006757 | 0.890909 | TRUE | 1 |
| ENST00000555725.1 | Testicular.Germ.Cell.Tumor | −8.61534 | −9.00248 | −3.16317 | 0 | 0 | Down | Testis | 0.006757 | 0.90303 | TRUE | 1 |
| ENST00000448198.2 | Testicular.Germ.Cell.Tumor | −8.10773 | −8.62919 | −3.6049 | 0 | 0 | Down | Testis | 0.006757 | 0.878788 | TRUE | 1 |
| ENST00000435973.1 | Testicular.Germ.Cell.Tumor | −8.08735 | −9.18418 | −3.91616 | 0 | 0 | Down | Testis | 0 | 0.884848 | TRUE | 1 |
| ENST00000504766.1 | Testicular.Germ.Cell.Tumor | −7.38491 | −8.21602 | −4.03338 | 0 | 0 | Down | Testis | 0 | 0.727273 | TRUE | 1 |
| ENST00000435444.1 | Testicular.Germ.Cell.Tumor | −7.2011 | −7.99844 | −4.05444 | 3.2046069129245e−310 | 4.15E−308 | Down | Testis | 0 | 0.739394 | TRUE | 1 |
| ENST00000505650.2 | Testicular.Germ.Cell.Tumor | −8.1066 | −8.32701 | −3.03311 | 2.30E−282 | 2.39E−280 | Down | Testis | 0.006757 | 0.90303 | TRUE | 1 |
| ENST00000456163.1 | Testicular.Germ.Cell.Tumor | −9.05378 | −9.09314 | −0.3284 | 2.89E−281 | 2.96E−279 | Down | Testis | 0.013514 | 0.921212 | TRUE | 1 |
| ENST00000275590.9 | Testicular.Germ.Cell.Tumor | −5.47725 | −5.48564 | −0.55273 | 1.83E−210 | 1.09E−208 | Down | Testis | 0.27027 | 0.993939 | TRUE | 1 |
| ENST00000568500.1 | Testicular.Germ.Cell.Tumor | −4.12368 | −4.12842 | 0.025228 | 4.89E−203 | 2.73E−201 | Down | Testis | 0.682432 | 0.993939 | TRUE | 1 |
| ENST00000467017.1 | Testicular.Germ.Cell.Tumor | −6.99544 | −7.59782 | −4.01394 | 2.72E−198 | 1.46E−196 | Down | Testis | 0 | 0.745455 | TRUE | 1 |
| ENST00000563807.1 | Testicular.Germ.Cell.Tumor | −8.45291 | −8.98677 | −3.40494 | 3.27E−192 | 1.68E−190 | Down | Testis | 0 | 0.89697 | TRUE | 1 |
| ENST00000500741.2 | Testicular.Germ.Cell.Tumor | −3.3819 | −3.38271 | 2.859783 | 7.64E−177 | 3.50E−175 | Down | Testis | 1 | 1 | TRUE | 1 |
| ENST00000566232.1 | Testicular.Germ.Cell.Tumor | −8.51637 | −8.88962 | −2.72993 | 1.73E−174 | 7.77E−173 | Down | Testis | 0 | 0.90303 | TRUE | 1 |
| ENST00000546421.1 | Testicular.Germ.Cell.Tumor | −7.32784 | −7.75988 | −3.796 | 7.94E−164 | 3.26E−162 | Down | Testis | 0 | 0.739394 | TRUE | 1 |
| ENST00000598832.1 | Testicular.Germ.Cell.Tumor | −7.44757 | −7.72148 | −3.44848 | 2.05E−154 | 7.79E−153 | Down | Testis | 0 | 0.890909 | TRUE | 1 |
| ENST00000445088.1 | Testicular.Germ.Cell.Tumor | −7.68435 | −7.71219 | −1.05436 | 1.32E−136 | 4.27E−135 | Down | Testis | 0.033784 | 0.945455 | TRUE | 1 |
| ENST00000489259.5 | Testicular.Germ.Cell.Tumor | −6.34285 | −6.39404 | −2.35728 | 1.95E−132 | 6.05E−131 | Down | Testis | 0.006757 | 0.90303 | TRUE | 1 |
| ENST00000606496.1 | Testicular.Germ.Cell.Tumor | −3.25426 | −3.25862 | 0.947714 | 2.56E−124 | 7.24E−123 | Down | Testis | 0.702703 | 0.993939 | TRUE | 1 |
| ENST00000412690.1 | Testicular.Germ.Cell.Tumor | −6.77326 | −6.78198 | −0.38754 | 1.27E−119 | 3.44E−118 | Down | Testis | 0.22973 | 0.957576 | TRUE | 1 |
| ENST00000438431.1 | Testicular.Germ.Cell.Tumor | −6.60728 | −7.02649 | −3.93821 | 4.05E−118 | 1.08E−116 | Down | Testis | 0 | 0.739394 | TRUE | 1 |
| ENST00000510795.1 | Testicular.Germ.Cell.Tumor | −7.34006 | −7.71008 | −1.5668 | 8.67E−116 | 2.26E−114 | Down | Testis | 0 | 0.890909 | TRUE | 1 |
| ENST00000382849.2 | Testicular.Germ.Cell.Tumor | −6.21777 | −6.2784 | −1.62411 | 2.58E−111 | 6.38E−110 | Down | Testis | 0.02027 | 0.945455 | TRUE | 1 |
| ENST00000424312.2 | Testicular.Germ.Cell.Tumor | −6.53029 | −6.68353 | −3.48661 | 1.21E−107 | 2.87E−106 | Down | Testis | 0 | 0.89697 | TRUE | 1 |
| ENST00000612584.1 | Testicular.Germ.Cell.Tumor | −6.569 | −6.74807 | −2.9297 | 8.17E−103 | 1.84E−101 | Down | Testis | 0 | 0.872727 | TRUE | 1 |
| ENST00000435478.1 | Testicular.Germ.Cell.Tumor | −7.05519 | −7.46665 | −3.70388 | 5.57E−102 | 1.24E−100 | Down | Testis | 0 | 0.866667 | TRUE | 1 |
| ENST00000452229.1 | Testicular.Germ.Cell.Tumor | −7.33702 | −7.60902 | −3.43423 | 6.70E−101 | 1.47E−99 | Down | Testis | 0 | 0.884848 | TRUE | 1 |
| ENST00000560864.1 | Testicular.Germ.Cell.Tumor | −6.98039 | −7.67536 | −3.9094 | 5.54E−96 | 1.14E−94 | Down | Testis | 0 | 0.818182 | TRUE | 1 |
| ENST00000417786.1 | Testicular.Germ.Cell.Tumor | −5.86262 | −5.97689 | −2.79449 | 2.58E−91 | 4.99E−90 | Down | Testis | 0 | 0.915152 | TRUE | 1 |
| ENST00000443576.3 | Testicular.Germ.Cell.Tumor | −6.50731 | −7.03473 | −3.89299 | 2.62E−90 | 5.02E−89 | Down | Testis | 0 | 0.70303 | TRUE | 1 |
| ENST00000527594.1 | Testicular.Germ.Cell.Tumor | −5.62176 | −5.73254 | −3.07632 | 3.01E−88 | 5.60E−87 | Down | Testis | 0 | 0.878788 | TRUE | 1 |
| ENST00000502221.2 | Testicular.Germ.Cell.Tumor | −5.00441 | −5.03085 | −3.0186 | 1.51E−81 | 2.54E−80 | Down | Testis | 0.033784 | 0.939394 | TRUE | 1 |
| ENST00000608817.2 | Testicular.Germ.Cell.Tumor | −6.10818 | −6.12761 | −1.72702 | 4.57E−77 | 7.12E−76 | Down | Testis | 0.060811 | 0.957576 | TRUE | 1 |
| ENST00000551421.1 | Testicular.Germ.Cell.Tumor | −3.26156 | −3.26467 | −0.09373 | 2.27E−70 | 3.17E−69 | Down | Testis | 0.756757 | 0.975758 | TRUE | 1 |
| ENST00000621561.4 | Testicular.Germ.Cell.Tumor | −6.15884 | −6.19829 | −2.4097 | 3.21E−66 | 4.20E−65 | Down | Testis | 0.027027 | 0.909091 | TRUE | 1 |
| ENST00000615826.1 | Testicular.Germ.Cell.Tumor | −7.11382 | −7.33475 | −1.74761 | 1.34E−65 | 1.74E−64 | Down | Testis | 0.006757 | 0.733333 | TRUE | 1 |
| ENST00000608612.1 | Testicular.Germ.Cell.Tumor | −4.31285 | −4.38205 | −2.11387 | 3.26E−65 | 4.18E−64 | Down | Testis | 0 | 0.806061 | TRUE | 1 |
| ENST00000530002.1 | Testicular.Germ.Cell.Tumor | −5.66045 | −5.80826 | −3.0061 | 4.59E−64 | 5.79E−63 | Down | Testis | 0 | 0.842424 | TRUE | 1 |
| ENST00000480546.1 | Testicular.Germ.Cell.Tumor | −6.33701 | −6.35246 | −1.23576 | 1.85E−63 | 2.32E−62 | Down | Testis | 0.067568 | 0.945455 | TRUE | 1 |
| ENST00000439875.1 | Testicular.Germ.Cell.Tumor | −5.5975 | −5.61012 | −0.1751 | 8.22E−62 | 9.96E−61 | Down | Testis | 0.162162 | 0.981818 | TRUE | 1 |
| ENST00000553102.1 | Testicular.Germ.Cell.Tumor | −4.11029 | −4.20356 | −2.88461 | 3.35E−52 | 3.37E−51 | Down | Testis | 0 | 0.715152 | TRUE | 1 |
| ENST00000538355.1 | Testicular.Germ.Cell.Tumor | −4.23449 | −4.25328 | −1.96547 | 2.59E−48 | 2.41E−47 | Down | Testis | 0.047297 | 0.90303 | TRUE | 1 |
| ENST00000452565.1 | Testicular.Germ.Cell.Tumor | −2.80044 | −2.80798 | −0.54828 | 3.07E−46 | 2.74E−45 | Down | Testis | 0.304054 | 0.933333 | TRUE | 1 |
| ENST00000444843.1 | Testicular.Germ.Cell.Tumor | −4.27613 | −4.28149 | −1.39331 | 4.89E−46 | 4.35E−45 | Down | Testis | 0.506757 | 0.933333 | TRUE | 1 |
| ENST00000428752.1 | Testicular.Germ.Cell.Tumor | −4.61476 | −4.66294 | −2.25096 | 2.15E−45 | 1.89E−44 | Down | Testis | 0.013514 | 0.890909 | TRUE | 1 |
| ENST00000577297.5 | Testicular.Germ.Cell.Tumor | −3.75455 | −3.76855 | −2.07446 | 1.27E−43 | 1.08E−42 | Down | Testis | 0.094595 | 0.915152 | TRUE | 1 |
| ENST00000453554.1 | Testicular.Germ.Cell.Tumor | −4.77764 | −4.80531 | −1.8054 | 7.49E−42 | 6.10E−41 | Down | Testis | 0.027027 | 0.90303 | TRUE | 1 |
| ENST00000607801.5 | Testicular.Germ.Cell.Tumor | −3.57286 | −3.62303 | −2.04325 | 2.34E−39 | 1.80E−38 | Down | Testis | 0.006757 | 0.824242 | TRUE | 1 |
| ENST00000454280.1 | Testicular.Germ.Cell.Tumor | −3.11825 | −3.12162 | −1.60013 | 8.27E−39 | 6.26E−38 | Down | Testis | 0.47973 | 0.951515 | TRUE | 1 |
| ENST00000435039.3 | Testicular.Germ.Cell.Tumor | −4.14071 | −4.15999 | −1.4378 | 1.93E−37 | 1.42E−36 | Down | Testis | 0.074324 | 0.90303 | TRUE | 1 |
| ENST00000605945.1 | Testicular.Germ.Cell.Tumor | −2.32955 | −2.3346 | −0.47864 | 3.06E−37 | 2.23E−36 | Down | Testis | 0.594595 | 0.945455 | TRUE | 1 |
| ENST00000456582.1 | Testicular.Germ.Cell.Tumor | −3.24994 | −3.28306 | −1.32862 | 5.47E−37 | 3.97E−36 | Down | Testis | 0.02027 | 0.842424 | TRUE | 1 |
| ENST00000504756.1 | Testicular.Germ.Cell.Tumor | −3.42233 | −3.44307 | −2.047 | 6.71E−33 | 4.39E−32 | Down | Testis | 0.027027 | 0.90303 | TRUE | 1 |
| ENST00000449426.1 | Testicular.Germ.Cell.Tumor | −2.91665 | −2.93974 | −2.62155 | 1.23E−32 | 7.98E−32 | Down | Testis | 0 | 0.787879 | TRUE | 1 |
| ENST00000528023.3 | Testicular.Germ.Cell.Tumor | −2.46068 | −2.46671 | −1.27701 | 1.34E−31 | 8.47E−31 | Down | Testis | 0.385135 | 0.909091 | TRUE | 1 |
| ENST00000556328.1 | Testicular.Germ.Cell.Tumor | −2.4172 | −2.43772 | −1.80022 | 3.27E−29 | 1.94E−28 | Down | Testis | 0.02027 | 0.775758 | TRUE | 1 |
| ENST00000588144.2 | Testicular.Germ.Cell.Tumor | −2.53753 | −2.54791 | −1.84656 | 2.99E−28 | 1.73E−27 | Down | Testis | 0.114865 | 0.927273 | TRUE | 1 |
| ENST00000382988.3 | Testicular.Germ.Cell.Tumor | −3.38787 | −3.4203 | −3.6014 | 3.02E−26 | 1.66E−25 | Down | Testis | 0.027027 | 0.939394 | TRUE | 1 |
| ENST00000433544.1 | Testicular.Germ.Cell.Tumor | −2.73537 | −2.75212 | −1.26997 | 6.52E−24 | 3.35E−23 | Down | Testis | 0.027027 | 0.842424 | TRUE | 1 |
| ENST00000607997.1 | Testicular.Germ.Cell.Tumor | −2.30035 | −2.32195 | −2.12986 | 2.78E−23 | 1.40E−22 | Down | Testis | 0.013514 | 0.781818 | TRUE | 1 |
| ENST00000504989.1 | Testicular.Germ.Cell.Tumor | −4.24428 | −4.25824 | −1.5725 | 9.32E−23 | 4.62E−22 | Down | Testis | 0.141892 | 0.969697 | TRUE | 1 |
| ENST00000427188.1 | Testicular.Germ.Cell.Tumor | −3.24812 | −3.28398 | −1.42557 | 1.31E−22 | 6.48E−22 | Down | Testis | 0 | 0.927273 | TRUE | 1 |
| ENST00000501133.2 | Testicular.Germ.Cell.Tumor | −1.72921 | −1.73333 | −0.19471 | 2.46E−22 | 1.20E−21 | Down | Testis | 0.709459 | 0.987879 | TRUE | 1 |
| ENST00000570077.1 | Testicular.Germ.Cell.Tumor | −3.56167 | −3.59224 | −2.81332 | 8.94E−22 | 4.29E−21 | Down | Testis | 0.054054 | 0.884848 | TRUE | 1 |
| ENST00000444924.1 | Testicular.Germ.Cell.Tumor | −3.67581 | −3.7461 | −3.64178 | 5.85E−21 | 2.74E−20 | Down | Testis | 0.006757 | 0.70303 | TRUE | 1 |
| ENST00000623970.1 | Testicular.Germ.Cell.Tumor | −1.63669 | −1.6384 | 1.600032 | 2.95E−20 | 1.35E−19 | Down | Testis | 0.993243 | 1 | TRUE | 1 |
| ENST00000612344.1 | Testicular.Germ.Cell.Tumor | −3.20497 | −3.20701 | −1.5456 | 2.39E−18 | 1.02E−17 | Down | Testis | 0.601351 | 0.963636 | TRUE | 1 |
| ENST00000434051.1 | Testicular.Germ.Cell.Tumor | −3.60517 | −3.60989 | −0.15965 | 4.94E−18 | 2.10E−17 | Down | Testis | 0.418919 | 0.981818 | TRUE | 1 |
| ENST00000565523.1 | Testicular.Germ.Cell.Tumor | −2.56044 | −2.57386 | −3.68467 | 9.69E−17 | 3.92E−16 | Down | Testis | 0.121622 | 0.854545 | TRUE | 1 |
| ENST00000437593.1 | Testicular.Germ.Cell.Tumor | −2.30525 | −2.31402 | −0.79003 | 7.73E−15 | 2.92E−14 | Down | Testis | 0.209459 | 0.963636 | TRUE | 1 |
| ENST00000383686.2 | Testicular.Germ.Cell.Tumor | −2.73246 | −2.74676 | −2.43329 | 2.50E−14 | 9.25E−14 | Down | Testis | 0.027027 | 0.933333 | TRUE | 1 |
| ENST00000585890.1 | Testicular.Germ.Cell.Tumor | −2.84199 | −2.84969 | −1.59409 | 4.45E−14 | 1.63E−13 | Down | Testis | 0.283784 | 0.945455 | TRUE | 1 |
| ENST00000457998.2 | Testicular.Germ.Cell.Tumor | −1.62877 | −1.64097 | 0.298637 | 5.72E−13 | 2.00E−12 | Down | Testis | 0.074324 | 0.818182 | TRUE | 1 |
| ENST00000454346.1 | Testicular.Germ.Cell.Tumor | −1.72845 | −1.73842 | −1.47387 | 7.19E−13 | 2.50E−12 | Down | Testis | 0.033784 | 0.842424 | TRUE | 1 |
| ENST00000423121.1 | Testicular.Germ.Cell.Tumor | −1.84791 | −1.86053 | −1.72099 | 2.43E−12 | 8.26E−12 | Down | Testis | 0.087838 | 0.824242 | TRUE | 1 |
| ENST00000445070.1 | Testicular.Germ.Cell.Tumor | −2.32394 | −2.33314 | −2.01774 | 3.45E−12 | 1.17E−11 | Down | Testis | 0.263514 | 0.866667 | TRUE | 1 |
| ENST00000597336.1 | Testicular.Germ.Cell.Tumor | −1.70113 | −1.70558 | −0.14198 | 3.76E−12 | 1.27E−11 | Down | Testis | 0.486486 | 0.975758 | TRUE | 1 |
| ENST00000498979.6 | Testicular.Germ.Cell.Tumor | −2.20626 | −2.21492 | −1.36554 | 1.19E−11 | 3.94E−11 | Down | Testis | 0.22973 | 0.89697 | TRUE | 1 |
| ENST00000405916.2 | Testicular.Germ.Cell.Tumor | −2.25404 | −2.26704 | −3.22926 | 4.37E−11 | 1.41E−10 | Down | Testis | 0.168919 | 0.90303 | TRUE | 1 |
| ENST00000570025.1 | Testicular.Germ.Cell.Tumor | −1.60276 | −1.60768 | −0.37332 | 4.96E−11 | 1.59E−10 | Down | Testis | 0.412162 | 0.927273 | TRUE | 1 |
| ENST00000454117.1 | Testicular.Germ.Cell.Tumor | −2.00132 | −2.01897 | −0.91859 | 9.04E−11 | 2.87E−10 | Down | Testis | 0.040541 | 0.757576 | TRUE | 1 |
| ENST00000581181.5 | Testicular.Germ.Cell.Tumor | −2.29637 | −2.31299 | −1.50295 | 5.52E−10 | 1.69E−09 | Down | Testis | 0.081081 | 0.842424 | TRUE | 1 |
| ENST00000606878.1 | Testicular.Germ.Cell.Tumor | −1.75172 | −1.7654 | −1.55726 | 1.89E−09 | 5.63E−09 | Down | Testis | 0.040541 | 0.836364 | TRUE | 1 |
| ENST00000437258.5 | Testicular.Germ.Cell.Tumor | −2.23203 | −2.23888 | −1.11324 | 2.50E−09 | 7.41E−09 | Down | Testis | 0.195946 | 0.872727 | TRUE | 1 |
| ENST00000393023.2 | Testicular.Germ.Cell.Tumor | −2.17941 | −2.19294 | −3.84975 | 2.86E−09 | 8.47E−09 | Down | Testis | 0.081081 | 0.854545 | TRUE | 1 |
| ENST00000607333.1 | Testicular.Germ.Cell.Tumor | −1.94697 | −1.95309 | −0.71862 | 1.11E−08 | 3.18E−08 | Down | Testis | 0.162162 | 0.921212 | TRUE | 1 |
| ENST00000559120.1 | Testicular.Germ.Cell.Tumor | −2.0568 | −2.06937 | −3.76353 | 3.42E−08 | 9.60E−08 | Down | Testis | 0.121622 | 0.836364 | TRUE | 1 |
| ENST00000625168.1 | Testicular.Germ.Cell.Tumor | −1.45915 | −1.46015 | 1.148603 | 3.89E−08 | 1.09E−07 | Down | Testis | 0.986486 | 1 | TRUE | 1 |
| ENST00000469070.1 | Testicular.Germ.Cell.Tumor | −1.48681 | −1.49273 | −1.42307 | 5.03E−07 | 1.32E−06 | Down | Testis | 0.297297 | 0.969697 | TRUE | 1 |
| ENST00000432807.1 | Testicular.Germ.Cell.Tumor | −1.65483 | −1.6573 | 1.170656 | 6.64E−07 | 1.73E−06 | Down | Testis | 0.783784 | 0.993939 | TRUE | 1 |
| ENST00000501143.1 | Testicular.Germ.Cell.Tumor | −1.61048 | −1.61521 | −2.1715 | 2.36E−06 | 5.96E−06 | Down | Testis | 0.398649 | 0.951515 | TRUE | 1 |
| ENST00000551108.1 | Testicular.Germ.Cell.Tumor | −1.98839 | −1.99883 | −3.36144 | 7.70E−06 | 1.88E−05 | Down | Testis | 0.141892 | 0.878788 | TRUE | 1 |
| ENST00000441932.1 | Testicular.Germ.Cell.Tumor | −1.3967 | −1.40184 | 0.588765 | 1.13E−05 | 2.74E−05 | Down | Testis | 0.304054 | 0.963636 | TRUE | 1 |
| ENST00000591866.2 | Testicular.Germ.Cell.Tumor | −1.53073 | −1.54057 | −1.53556 | 1.65E−05 | 3.94E−05 | Down | Testis | 0.108108 | 0.878788 | TRUE | 1 |
| ENST00000427863.1 | Testicular.Germ.Cell.Tumor | −1.66454 | −1.67093 | −0.46263 | 2.47E−05 | 5.84E−05 | Down | Testis | 0.405405 | 0.909091 | TRUE | 1 |
| ENST00000561423.2 | Testicular.Germ.Cell.Tumor | −1.47485 | −1.48287 | −1.0371 | 9.58E−05 | 2.17E−04 | Down | Testis | 0.141892 | 0.909091 | TRUE | 1 |
| ENST00000548760.2 | Testicular.Germ.Cell.Tumor | −1.30784 | −1.30812 | 1.275759 | 2.10E−04 | 4.65E−04 | Down | Testis | 1 | 1 | TRUE | 1 |
| ENST00000611425.1 | Testicular.Germ.Cell.Tumor | −1.7015 | −1.70483 | −2.76232 | 2.50E−04 | 5.49E−04 | Down | Testis | 0.324324 | 0.890909 | TRUE | 1 |
| ENST00000441399.2 | Uterine.Carcinosarcoma | −2.57614 | −2.57957 | 0.735314 | 1.94E−24 | 4.00E−23 | Down | Uterus | 0.701754 | 1 | TRUE | 1 |
| ENST00000457601.1 | Uterine.Carcinosarcoma | −2.19664 | −2.21385 | −0.93472 | 5.47E−06 | 1.99E−05 | Down | Uterus | 0.052632 | 0.794872 | TRUE | 1 |

TABLE 2

| | | | tags.un- | | | | decide- | | prop_ex- | prop_ex- | is.freq.ex- | |
|---|---|---|---|---|---|---|---|---|---|---|---|---|
| transcript | disease | tags.logFC | shrunk.logFC | tags.logCPM | tags.PValue | tags.FDR | test | tissue | pressed_disease | pressed_tissue | pressed | n |
| ENST00000399586.2 | Breast.Invasive.Carcinoma | 1.266799 | 1.267379 | 2.455588 | 1.37E−04 | 3.47E−04 | Up | Breast | 0.980769 | 1 | TRUE | 1 |
| ENST00000522600.1 | Colon.Adenocarcinoma | 2.337505 | 2.356744 | −1.91549 | 1.28E−24 | 9.57E−24 | Up | Colon | 0.707317 | 0.100977 | TRUE | 1 |
| ENST00000555918.1 | Esophageal.Carcinoma | 2.071875 | 2.08131 | −1.01448 | 2.22E−52 | 3.00E−51 | Up | Esophagus | 0.745856 | 0.154908 | TRUE | 1 |
| ENST00000447334.1 | Glioblastoma.Multiforme | 2.425447 | 2.432156 | 0.759434 | 1.01E−70 | 1.13E−68 | Up | Brain | 0.947712 | 0.522648 | TRUE | 1 |
| ENST00000481651.1 | Glioblastoma.Multiforme | 2.456037 | 2.457348 | 2.287813 | 1.61E−67 | 1.64E−65 | Up | Brain | 0.993464 | 0.95122 | TRUE | 1 |
| ENST00000434063.3 | Glioblastoma.Multiforme | 2.876831 | 2.888343 | 0.347577 | 5.82E−56 | 3.89E−54 | Up | Brain | 0.75817 | 0.055749 | TRUE | 1 |
| ENST00000547851.1 | Glioblastoma.Multiforme | 1.998352 | 2.008634 | −1.01295 | 1.72E−19 | 2.36E−18 | Up | Brain | 0.738562 | 0.071429 | TRUE | 1 |
| ENST00000478818.1 | Glioblastoma.Multiforme | 1.832035 | 1.840467 | −1.22073 | 2.30E−14 | 2.29E−13 | Up | Brain | 0.718954 | 0.166376 | TRUE | 1 |
| ENST00000549565.1 | Glioblastoma.Multiforme | 1.330759 | 1.335233 | −0.48969 | 6.93E−07 | 3.90E−06 | Up | Brain | 0.888889 | 0.392857 | TRUE | 1 |
| ENST00000514146.1 | Liver.Hepatocellular.Carcinoma | 1.586355 | 1.58789 | 2.689413 | 8.22E−15 | 8.05E−14 | Up | Liver | 1 | 0.990909 | TRUE | 1 |
| ENST00000606089.1 | Liver.Hepatocellular.Carcinoma | 1.387358 | 1.393175 | −1.01608 | 3.96E−05 | 1.54E−04 | Up | Liver | 0.769648 | 0.363636 | TRUE | 1 |
| ENST00000593298.5 | Liver.Hepatocellular.Carcinoma | 1.847287 | 1.84815 | 1.857262 | 4.84E−05 | 1.86E−04 | Up | Liver | 0.766938 | 0.781818 | TRUE | 1 |
| ENST00000565118.1 | Lung.Adenocarcinoma | 1.954566 | 1.961693 | −0.66653 | 2.84E−14 | 1.38E−13 | Up | Lung | 0.773879 | 0.333333 | TRUE | 1 |
| ENST00000558388.6 | Lung.Adenocarcinoma | 2.0142 | 2.020875 | −0.82023 | 2.35E−12 | 1.05E−11 | Up | Lung | 0.834308 | 0.333333 | TRUE | 1 |
| ENST00000438290.2 | Lung.Squamous.Cell.Carcinoma | 6.148665 | 6.184055 | 1.540235 | 8.37E−140 | 6.02E−138 | Up | Lung | 0.875502 | 0.038194 | TRUE | 1 |
| ENST00000335142.5 | Lung.Squamous.Cell.Carcinoma | 3.098362 | 3.123215 | −0.86747 | 4.72E−74 | 8.70E−73 | Up | Lung | 0.74498 | 0 | TRUE | 1 |
| ENST00000412224.6 | Lung.Squamous.Cell.Carcinoma | 1.92986 | 1.935134 | 0.551161 | 6.27E−57 | 7.83E−56 | Up | Lung | 0.98996 | 0.590278 | TRUE | 1 |
| ENST00000441363.1 | Lung.Squamous.Cell.Carcinoma | 1.879991 | 1.887384 | 0.392702 | 3.78E−35 | 2.73E−34 | Up | Lung | 0.839357 | 0.291667 | TRUE | 1 |
| ENST00000414554.6 | Lung.Squamous.Cell.Carcinoma | 2.231759 | 2.246959 | −0.80667 | 2.33E−24 | 1.24E−23 | Up | Lung | 0.753012 | 0.097222 | TRUE | 1 |
| ENST00000429962.1 | Lung.Squamous.Cell.Carcinoma | 1.370724 | 1.373108 | 0.813275 | 6.01E−09 | 1.82E−08 | Up | Lung | 0.961847 | 0.819444 | TRUE | 1 |
| ENST00000426194.1 | Ovarian.Serous.Cystadenocarcinoma | 7.60561 | 7.810671 | −2.12283 | 7.23E−88 | 5.11E−86 | Up | Ovary | 0.966587 | 0 | TRUE | 1 |
| ENST00000358393.1 | Ovarian.Serous.Cystadenocarcinoma | 5.765467 | 5.784991 | −0.40257 | 3.48E−45 | 6.19E−44 | Up | Ovary | 0.909308 | 0.022727 | TRUE | 1 |
| ENST00000608013.1 | Ovarian.Serous.Cystadenocarcinoma | 5.070649 | 5.126281 | −1.18439 | 1.57E−36 | 1.98E−35 | Up | Ovary | 0.894988 | 0 | TRUE | 1 |
| ENST00000498979.6 | Ovarian.Serous.Cystadenocarcinoma | 3.915237 | 3.954691 | −1.36554 | 1.89E−34 | 2.18E−33 | Up | Ovary | 0.789976 | 0 | TRUE | 1 |
| ENST00000598755.1 | Ovarian.Serous.Cystadenocarcinoma | 2.767661 | 2.781434 | −1.16106 | 2.17E−26 | 1.75E−25 | Up | Ovary | 0.830549 | 0.045455 | TRUE | 1 |
| ENST00000471299.1 | Ovarian.Serous.Cystadenocarcinoma | 3.283173 | 3.303535 | −1.42297 | 1.61E−24 | 1.19E−23 | Up | Ovary | 0.811456 | 0.034091 | TRUE | 1 |
| ENST00000518831.1 | Ovarian.Serous.Cystadenocarcinoma | 2.726102 | 2.737257 | −1.5406 | 5.47E−22 | 3.61E−21 | Up | Ovary | 0.763723 | 0.090909 | TRUE | 1 |
| ENST00000608651.1 | Ovarian.Serous.Cystadenocarcinoma | 2.272966 | 2.275645 | 0.979449 | 1.48E−20 | 9.13E−20 | Up | Ovary | 0.973747 | 0.954545 | TRUE | 1 |
| ENST00000517833.1 | Ovarian.Serous.Cystadenocarcinoma | 2.480983 | 2.489693 | −0.72001 | 2.91E−11 | 1.09E−10 | Up | Ovary | 0.713604 | 0.25 | TRUE | 1 |
| ENST00000614292.1 | Ovarian.Serous.Cystadenocarcinoma | 1.852456 | 1.85996 | −1.01344 | 1.54E−10 | 5.53E−10 | Up | Ovary | 0.880668 | 0.25 | TRUE | 1 |
| ENST00000529253.5 | Ovarian.Serous.Cystadenocarcinoma | 1.98225 | 1.984849 | 0.763589 | 1.11E−09 | 3.78E−09 | Up | Ovary | 0.976134 | 0.784091 | TRUE | 1 |
| ENST00000398275.4 | Ovarian.Serous.Cystadenocarcinoma | 1.690947 | 1.693619 | 1.082225 | 6.66E−07 | 1.88E−06 | Up | Ovary | 0.933174 | 0.715909 | TRUE | 1 |
| ENST00000527620.5 | Ovarian.Serous.Cystadenocarcinoma | 1.900809 | 1.902744 | 2.168839 | 1.28E−06 | 3.54E−06 | Up | Ovary | 0.954654 | 0.909091 | TRUE | 1 |
| ENST00000437764.5 | Pancreatic.Adenocarcinoma | 3.373386 | 3.392231 | 0.71864 | 3.85E−55 | 2.05E−53 | Up | Pancreas | 0.910112 | 0.065868 | TRUE | 1 |
| ENST00000457107.5 | Pancreatic.Adenocarcinoma | 2.094801 | 2.098351 | −0.39667 | 7.77E−09 | 3.87E−08 | Up | Pancreas | 0.893258 | 0.712575 | TRUE | 1 |
| ENST00000608395.1 | Pancreatic.Adenocarcinoma | 1.601435 | 1.603567 | 4.075536 | 9.76E−08 | 4.41E−07 | Up | Pancreas | 0.994382 | 0.952096 | TRUE | 1 |
| ENST00000316124.3 | Prostate.Adenocarcinoma | 3.13455 | 3.145605 | −0.27753 | 2.87E−32 | 7.21E−31 | Up | Prostate | 0.88664 | 0.18 | TRUE | 1 |
| ENST00000548416.1 | Prostate.Adenocarcinoma | 3.981801 | 3.988493 | −0.78655 | 1.24E−29 | 2.69E−28 | Up | Prostate | 0.809717 | 0.34 | TRUE | 1 |
| ENST00000579458.1 | Prostate.Adenocarcinoma | 3.91606 | 3.918201 | 0.789008 | 4.24E−25 | 6.92E−24 | Up | Prostate | 0.95749 | 0.73 | TRUE | 1 |
| ENST00000414022.5 | Prostate.Adenocarcinoma | 3.875599 | 3.937004 | −1.91624 | 2.05E−22 | 2.83E−21 | Up | Prostate | 0.714575 | 0.01 | TRUE | 1 |
| ENST00000589518.1 | Prostate.Adenocarcinoma | 2.385254 | 2.394401 | −1.62453 | 3.74E−17 | 3.59E−16 | Up | Prostate | 0.902834 | 0.25 | TRUE | 1 |
| ENST00000467458.2 | Prostate.Adenocarcinoma | 1.885841 | 1.886884 | 1.758937 | 1.36E−13 | 9.93E−13 | Up | Prostate | 1 | 1 | TRUE | 1 |
| ENST00000576313.1 | Prostate.Adenocarcinoma | 2.04763 | 2.062647 | −1.98743 | 5.16E−11 | 3.07E−10 | Up | Prostate | 0.793522 | 0.07 | TRUE | 1 |
| ENST00000398832.2 | Prostate.Adenocarcinoma | 1.954896 | 1.964893 | −0.48283 | 3.96E−10 | 2.19E−09 | Up | Prostate | 0.836032 | 0.16 | TRUE | 1 |
| ENST00000510795.1 | Prostate.Adenocarcinoma | 2.259578 | 2.264495 | −1.5668 | 9.33E−10 | 4.99E−09 | Up | Prostate | 0.961538 | 0.48 | TRUE | 1 |
| ENST00000317114.1 | Skin.Cutaneous.Melanoma | 2.645879 | 2.649299 | 2.072076 | 1.81E−81 | 6.61E−80 | Up | Skin | 1 | 0.85045 | TRUE | 1 |
| ENST00000450133.5 | Skin.Cutaneous.Melanoma | 2.569003 | 2.580033 | −0.57787 | 1.66E−13 | 7.58E−13 | Up | Skin | 0.843137 | 0.223423 | TRUE | 1 |
| ENST00000427063.6 | Skin.Cutaneous.Melanoma | 1.384476 | 1.384888 | 3.346456 | 2.52E−09 | 9.00E−09 | Up | Skin | 1 | 1 | TRUE | 1 |
| ENST00000501133.2 | Skin.Cutaneous.Melanoma | 1.378402 | 1.383195 | −0.19471 | 5.09E−09 | 1.78E−08 | Up | Skin | 0.980392 | 0.520721 | TRUE | 1 |
| ENST00000444583.6 | Skin.Cutaneous.Melanoma | 1.63482 | 1.63628 | 1.44482 | 6.15E−07 | 1.87E−06 | Up | Skin | 0.980392 | 0.846847 | TRUE | 1 |
| ENST00000606723.2 | Skin.Cutaneous.Melanoma | 1.258983 | 1.259864 | 2.316787 | 1.96E−04 | 4.87E−04 | Up | Skin | 1 | 0.992793 | TRUE | 1 |
| ENST00000456333.2 | Stomach.Adenocarcinoma | 1.825226 | 1.836805 | −0.91965 | 9.05E−28 | 7.82E−27 | Up | Stomach | 0.745763 | 0.086207 | TRUE | 1 |
| ENST00000478808.2 | Stomach.Adenocarcinoma | 2.130083 | 2.13952 | −0.64873 | 8.11E−16 | 4.23E−15 | Up | Stomach | 0.709443 | 0.235632 | TRUE | 1 |
| ENST00000426200.1 | Stomach.Adenocarcinoma | 1.419262 | 1.420469 | 1.712905 | 8.67E−06 | 2.53E−05 | Up | Stomach | 0.985472 | 0.942529 | TRUE | 1 |
| ENST00000505632.1 | Testicular.Germ.Cell.Tumor | 7.206516 | 7.365795 | −3.25489 | 2.08E−181 | 9.86E−180 | Up | Testis | 0.844595 | 0 | TRUE | 1 |
| ENST00000325042.2 | Testicular.Germ.Cell.Tumor | 5.041499 | 5.122812 | −2.06398 | 1.57E−60 | 1.86E−59 | Up | Testis | 0.756757 | 0 | TRUE | 1 |
| ENST00000592918.5 | Testicular.Germ.Cell.Tumor | 4.581959 | 4.584341 | 2.396674 | 2.92E−59 | 3.36E−58 | Up | Testis | 0.945946 | 0.957576 | TRUE | 1 |
| ENST00000591299.1 | Testicular.Germ.Cell.Tumor | 4.588225 | 4.62148 | −1.05679 | 3.73E−45 | 3.26E−44 | Up | Testis | 0.763514 | 0 | TRUE | 1 |
| ENST00000427501.5 | Testicular.Germ.Cell.Tumor | 2.604432 | 2.623247 | −0.89861 | 4.17E−25 | 2.22E−24 | Up | Testis | 0.75 | 0.012121 | TRUE | 1 |
| ENST00000449713.1 | Testicular.Germ.Cell.Tumor | 1.895307 | 1.897835 | −0.92621 | 5.61E−06 | 1.38E−05 | Up | Testis | 0.75 | 0.915152 | TRUE | 1 |
| ENST00000424846.3 | Testicular.Germ.Cell.Tumor | 1.275645 | 1.27626 | 2.68932 | 1.45E−04 | 3.25E−04 | Up | Testis | 1 | 1 | TRUE | 1 |
| ENST00000583271.5 | Testicular.Germ.Cell.Tumor | 1.280313 | 1.280435 | 5.002368 | 2.05E−04 | 4.54E−04 | Up | Testis | 1 | 1 | TRUE | 1 |
| ENST00000456481.1 | Uterine.Carcinosarcoma | 3.101251 | 3.124663 | −1.80802 | 1.08E−25 | 2.46E−24 | Up | Uterus | 0.789474 | 0.012821 | TRUE | 1 |
| ENST00000587506.1 | Uterine.Carcinosarcoma | 1.719483 | 1.7249 | −0.61675 | 1.25E−07 | 5.52E−07 | Up | Uterus | 0.807018 | 0.538462 | TRUE | 1 |
| ENST00000588226.5 | Uterine.Carcinosarcoma | 1.709931 | 1.713284 | 0.049321 | 5.99E−05 | 1.90E−04 | Up | Uterus | 0.859649 | 0.820513 | TRUE | 1 |

TABLE 3

| | | | tags.un- | | | | decide- | | prop_ex- | prop_ex- | is.freq.ex- | |
|---|---|---|---|---|---|---|---|---|---|---|---|---|
| transcript | disease | tags.logFC | shrunk.logFC | tags.logCPM | tags.PValue | tags.FDR | test | tissue | pressed_disease | pressed_tissue | pressed | n |
| ENST00000523301.1 | Breast.Invasive.Carcinoma | −3.00641 | −3.01676 | −1.68755 | 4.37E−69 | 7.70E−67 | Down | Breast | 0.124542 | 0.734513 | TRUE | 1 |
| ENST00000594624.6 | Breast.Invasive.Carcinoma | −2.95318 | −2.96235 | −1.81582 | 6.08E−46 | 6.44E−44 | Down | Breast | 0.17674 | 0.902655 | TRUE | 1 |
| ENST00000530595.1 | Breast.Invasive.Carcinoma | −1.57974 | −1.58873 | −1.80367 | 5.04E−06 | 4.36E−05 | Down | Breast | 0.087912 | 0.761062 | TRUE | 1 |
| ENST00000428939.3 | Breast.Invasive.Carcinoma | −1.57099 | −1.57599 | 0.0112 | 6.56E−06 | 5.59E−05 | Down | Breast | 0.261905 | 0.876106 | TRUE | 1 |
| ENST00000426483.1 | Colon.Adenocarcinoma | −2.68415 | −2.69984 | −2.77211 | 3.82E−19 | 5.58E−17 | Down | Colon | 0.020906 | 0.829268 | TRUE | 1 |
| ENST00000420367.1 | Colon.Adenocarcinoma | −1.63393 | −1.63614 | 0.582774 | 4.29E−08 | 1.11E−06 | Down | Colon | 0.885017 | 1 | TRUE | 1 |
| ENST00000452079.5 | Colon.Adenocarcinoma | −1.49826 | −1.49913 | 3.602258 | 2.87E−05 | 3.78E−04 | Down | Colon | 0.878049 | 1 | TRUE | 1 |
| ENST00000377722.2 | Kidney.Chromophobe | −6.16682 | −6.23613 | 1.244532 | 6.33E−33 | 6.76E−31 | Down | Kidney | 0.030303 | 0.899225 | TRUE | 1 |
| ENST00000500741.2 | Kidney.Chromophobe | −1.3783 | −1.37871 | 3.256661 | 1.33E−06 | 1.30E−05 | Down | Kidney | 1 | 1 | TRUE | 1 |
| ENST00000447668.2 | Kidney.Chromophobe | −1.52601 | −1.52734 | 1.226064 | 2.44E−06 | 2.29E−05 | Down | Kidney | 0.924242 | 1 | TRUE | 1 |
| ENST00000602367.1 | Kidney.Chromophobe | −1.73983 | −1.7411 | 2.82483 | 1.74E−05 | 1.41E−04 | Down | Kidney | 0.833333 | 1 | TRUE | 1 |
| ENST00000397841.5 | Kidney.Chromophobe | −1.45074 | −1.45335 | 0.607343 | 1.01E−04 | 7.14E−04 | Down | Kidney | 0.530303 | 0.992248 | TRUE | 1 |
| ENST00000612598.1 | Kidney.Clear.Cell.Carcinoma | −1.56786 | −1.56908 | 2.49122 | 4.30E−12 | 5.36E−11 | Down | Kidney | 0.983019 | 1 | TRUE | 1 |
| ENST00000592918.5 | Kidney.Clear.Cell.Carcinoma | −2.00093 | −2.00217 | 2.004612 | 3.64E−08 | 3.02E−07 | Down | Kidney | 0.573585 | 1 | TRUE | 1 |
| ENST00000522674.1 | Kidney.Clear.Cell.Carcinoma | −2.06729 | −2.07301 | 1.801662 | 7.54E−08 | 6.01E−07 | Down | Kidney | 0.230189 | 0.984496 | TRUE | 1 |
| ENST00000458624.1 | Kidney.Clear.Cell.Carcinoma | −1.77935 | −1.78815 | 0.789754 | 3.47E−07 | 2.56E−06 | Down | Kidney | 0.084906 | 0.782946 | TRUE | 1 |
| ENST00000541704.2 | Kidney.Clear.Cell.Carcinoma | −1.52802 | −1.53279 | −1.87011 | 1.39E−05 | 8.31E−05 | Down | Kidney | 0.286792 | 0.899225 | TRUE | 1 |
| ENST00000501079.5 | Kidney.Clear.Cell.Carcinoma | −1.22676 | −1.22696 | 3.187274 | 1.74E−04 | 8.81E−04 | Down | Kidney | 1 | 1 | TRUE | 1 |
| ENST00000531871.3 | Kidney.Papillary.Cell.Carcinoma | −2.08875 | −2.09566 | −0.39188 | 3.85E−05 | 2.12E−04 | Down | Kidney | 0.184028 | 0.72093 | TRUE | 1 |
| ENST00000421685.2 | Kidney.Papillary.Cell.Carcinoma | −1.2452 | −1.24602 | 1.622244 | 5.69E−05 | 3.05E−04 | Down | Kidney | 0.993056 | 1 | TRUE | 1 |
| ENST00000576252.1 | Liver.Hepatocellular.Carcinoma | −2.4547 | −2.45838 | −2.07179 | 1.88E−09 | 7.97E−08 | Down | Liver | 0.268293 | 0.8 | TRUE | 1 |
| ENST00000517833.1 | Liver.Hepatocellular.Carcinoma | −2.22911 | −2.23775 | −1.0499 | 1.08E−06 | 2.45E−05 | Down | Liver | 0.159892 | 0.82 | TRUE | 1 |
| ENST00000466734.5 | Lung.Adenocarcinoma | −1.34872 | −1.34956 | 1.843548 | 7.07E−05 | 5.08E−04 | Down | Lung | 0.966862 | 1 | TRUE | 1 |
| ENST00000577176.1 | Lung.Squamous.Cell.Carcinoma | −3.57575 | −3.61989 | −1.76147 | 4.05E−37 | 1.86E−35 | Down | Lung | 0.02008 | 0.770642 | TRUE | 1 |
| ENST00000599831.6 | Lung.Squamous.Cell.Carcinoma | −3.91664 | −3.92772 | −0.38443 | 5.19E−37 | 2.37E−35 | Down | Lung | 0.116466 | 0.963303 | TRUE | 1 |
| ENST00000426475.1 | Lung.Squamous.Cell.Carcinoma | −2.0209 | −2.02486 | −0.44337 | 2.24E−17 | 3.12E−16 | Down | Lung | 0.447791 | 1 | TRUE | 1 |
| ENST00000429172.5 | Lung.Squamous.Cell.Carcinoma | −2.03874 | −2.04826 | −1.59602 | 2.12E−14 | 2.42E−13 | Down | Lung | 0.128514 | 0.908257 | TRUE | 1 |
| ENST00000577066.2 | Lung.Squamous.Cell.Carcinoma | −1.48973 | −1.48986 | 4.695719 | 3.44E−13 | 3.59E−12 | Down | Lung | 1 | 1 | TRUE | 1 |
| ENST00000478808.2 | Lung.Squamous.Cell.Carcinoma | −1.73226 | −1.73525 | 0.035784 | 8.63E−08 | 5.69E−07 | Down | Lung | 0.516064 | 1 | TRUE | 1 |
| ENST00000609497.5 | Lung.Squamous.Cell.Carcinoma | −1.51583 | −1.51882 | 0.797076 | 1.87E−07 | 1.20E−06 | Down | Lung | 0.53012 | 1 | TRUE | 1 |
| ENST00000438210.1 | Lung.Squamous.Cell.Carcinoma | −1.6896 | −1.69565 | −1.76806 | 1.52E−06 | 8.79E−06 | Down | Lung | 0.220884 | 0.853211 | TRUE | 1 |
| ENST00000507794.2 | Lung.Squamous.Cell.Carcinoma | −1.37292 | −1.3761 | 0.019125 | 3.18E−05 | 1.58E−04 | Down | Lung | 0.508032 | 0.954128 | TRUE | 1 |
| ENST00000547851.1 | Lung.Squamous.Cell.Carcinoma | −1.49716 | −1.5018 | −1.24397 | 8.05E−05 | 3.80E−04 | Down | Lung | 0.327309 | 0.944954 | TRUE | 1 |
| ENST00000609153.1 | Stomach.Adenocarcinoma | −1.58131 | −1.5848 | −0.49467 | 1.46E−05 | 2.63E−04 | Down | Stomach | 0.447942 | 0.75 | TRUE | 1 |
| ENST00000438290.2 | Stomach.Adenocarcinoma | −2.28747 | −2.28837 | 0.798526 | 2.34E−05 | 3.98E−04 | Down | Stomach | 0.464891 | 0.777778 | TRUE | 1 |
| ENST00000500537.2 | Thyroid.Carcinoma | −2.3339 | −2.34812 | −3.32391 | 4.64E−07 | 1.75E−05 | Down | Thyroid | 0.107143 | 0.864407 | TRUE | 1 |
| | | | | | | | | Gland | | | | |
| ENST00000562172.2 | Uterine.Corpus.Endometrioid.Carcinoma | −2.82404 | −2.82593 | −0.75177 | 1.23E−11 | 8.31E−10 | Down | Endometrium | 0.838889 | 1 | TRUE | 1 |
| ENST00000437764.5 | Uterine.Corpus.Endometrioid.Carcinoma | −2.45985 | −2.46156 | 0.628165 | 3.72E−10 | 1.94E−08 | Down | Endometrium | 0.761111 | 1 | TRUE | 1 |
| ENST00000573951.1 | Uterine.Corpus.Endometrioid.Carcinoma | −1.63359 | −1.63716 | −0.01682 | 2.84E−06 | 5.40E−05 | Down | Endometrium | 0.55 | 1 | TRUE | 1 |
| ENST00000439875.1 | Uterine.Corpus.Endometrioid.Carcinoma | −2.3557 | −2.36337 | −0.72016 | 7.92E−05 | 9.24E−04 | Down | Endometrium | 0.2 | 0.869565 | TRUE | 1 |

TABLE 4

| | | | tags.un- | | | | decide- | | prop_ex- | prop_ex- | is.freq.ex- | |
|---|---|---|---|---|---|---|---|---|---|---|---|---|
| transcript | disease | tags.logFC | shrunk.logFC | tags.logCPM | tags.PValue | tags.FDR | test | tissue | pressed_disease | pressed_tissue | pressed | n |
| ENST00000559008.2 | Breast.Invasive.Carcinoma | 1.665734 | 1.66699 | 2.037803 | 1.16E−04 | 8.15E−04 | Up | Breast | 0.880952 | 0.823009 | TRUE | 1 |
| ENST00000500112.1 | Colon.Adenocarcinoma | 4.640765 | 4.648384 | 1.195102 | 3.03E−12 | 1.74E−10 | Up | Colon | 0.972125 | 0.195122 | TRUE | 1 |
| ENST00000411824.1 | Colon.Adenocarcinoma | 3.895436 | 3.905732 | −1.01074 | 9.22E−09 | 2.74E−07 | Up | Colon | 0.783972 | 0.04878 | TRUE | 1 |
| ENST00000417721.5 | Colon.Adenocarcinoma | 1.595769 | 1.596616 | 2.200341 | 4.80E−06 | 7.77E−05 | Up | Colon | 0.996516 | 1 | TRUE | 1 |
| ENST00000449500.1 | Colon.Adenocarcinoma | 1.590162 | 1.59219 | 1.335273 | 5.76E−05 | 6.99E−04 | Up | Colon | 0.989547 | 0.97561 | TRUE | 1 |
| ENST00000455557.2 | Head . . . Neck.SquamousCell.Carcinoma | 3.795759 | 3.80235 | 0.320549 | 8.36E−12 | 3.36E−10 | Up | Head and | 0.714286 | 0.090909 | TRUE | 1 |
| | | | | | | | | Neck region | | | | |
| ENST00000629441.1 | Head . . . Neck.SquamousCell.Carcinoma | 2.046502 | 2.050326 | 1.080903 | 4.74E−10 | 1.54E−08 | Up | Head and | 0.953668 | 0.386364 | TRUE | 1 |
| | | | | | | | | Neck region | | | | |
| ENST00000440326.1 | Head . . . Neck.SquamousCell.Carcinoma | 2.889075 | 2.897726 | −1.00815 | 1.20E−08 | 3.22E−07 | Up | Head and | 0.797297 | 0.181818 | TRUE | 1 |
| | | | | | | | | Neck region | | | | |
| ENST00000555918.1 | Head . . . Neck.SquamousCell.Carcinoma | 1.597474 | 1.603062 | −1.04289 | 1.21E−05 | 1.90E−04 | Up | Head and | 0.828185 | 0.204545 | TRUE | 1 |
| | | | | | | | | Neck region | | | | |
| ENST00000454935.1 | Kidney.Chromophobe | 2.974637 | 2.975434 | 2.671972 | 7.04E−54 | 2.46E−51 | Up | Kidney | 1 | 1 | TRUE | 1 |
| ENST00000412483.1 | Kidney.Chromophobe | 4.259647 | 4.282639 | −2.87174 | 2.49E−22 | 1.26E−20 | Up | Kidney | 0.742424 | 0.007752 | TRUE | 1 |
| ENST00000555562.1 | Kidney.Chromophobe | 2.166804 | 2.168153 | 1.192743 | 6.70E−18 | 2.34E−16 | Up | Kidney | 0.984848 | 1 | TRUE | 1 |
| ENST00000445184.1 | Kidney.Chromophobe | 1.695813 | 1.701129 | −0.4045 | 5.86E−07 | 6.08E−06 | Up | Kidney | 0.80303 | 0.263566 | TRUE | 1 |
| ENST00000499732.2 | Kidney.Chromophobe | 1.791219 | 1.791239 | 7.203578 | 3.15E−06 | 2.90E−05 | Up | Kidney | 1 | 1 | TRUE | 1 |
| ENST00000541196.2 | Kidney.Chromophobe | 1.642473 | 1.642562 | 5.092878 | 3.09E−05 | 2.40E−04 | Up | Kidney | 1 | 1 | TRUE | 1 |
| ENST00000586421.1 | Kidney.Chromophobe | 1.753202 | 1.757574 | −1.33018 | 5.89E−05 | 4.35E−04 | Up | Kidney | 0.757576 | 0.503876 | TRUE | 1 |
| ENST00000441184.1 | Kidney.Clear.Cell.Carcinoma | 9.154483 | 9.516906 | −1.16397 | 1.49E−111 | 6.56E−109 | Up | Kidney | 0.707547 | 0 | TRUE | 1 |
| ENST00000478818.1 | Kidney.Clear.Cell.Carcinoma | 5.149938 | 5.190719 | −1.10395 | 1.19E−95 | 3.74E−93 | Up | Kidney | 0.877358 | 0.007752 | TRUE | 1 |
| ENST00000609153.1 | Kidney.Clear.Cell.Carcinoma | 2.413685 | 2.41867 | −0.49467 | 1.21E−43 | 9.71E−42 | Up | Kidney | 0.943396 | 0.395349 | TRUE | 1 |
| ENST00000608794.1 | Kidney.Clear.Cell.Carcinoma | 2.97377 | 2.979017 | −0.55779 | 1.65E−39 | 1.13E−37 | Up | Kidney | 0.943396 | 0.434109 | TRUE | 1 |
| ENST00000481651.1 | Kidney.Clear.Cell.Carcinoma | 2.025692 | 2.026281 | 2.37328 | 6.45E−19 | 1.41E−17 | Up | Kidney | 0.996226 | 1 | TRUE | 1 |
| ENST00000591360.1 | Kidney.Clear.Cell.Carcinoma | 2.604186 | 2.61243 | −1.8961 | 2.95E−11 | 3.39E−10 | Up | Kidney | 0.786792 | 0.217054 | TRUE | 1 |
| ENST00000511543.1 | Kidney.Clear.Cell.Carcinoma | 1.626799 | 1.631377 | −0.45215 | 1.55E−08 | 1.34E−07 | Up | Kidney | 0.8 | 0.356589 | TRUE | 1 |
| ENST00000428939.3 | Kidney.Papillary.Cell.Carcinoma | 2.470259 | 2.474072 | 0.0112 | 8.15E−18 | 1.70E−16 | Up | Kidney | 0.854167 | 0.589147 | TRUE | 1 |
| ENST00000429630.1 | Kidney.Papillary.Cell.Carcinoma | 1.434365 | 1.434969 | 1.717205 | 3.85E−07 | 2.77E−06 | Up | Kidney | 1 | 1 | TRUE | 1 |
| ENST00000568654.1 | Kidney.Papillary.Cell.Carcinoma | 1.624048 | 1.626416 | 0.937888 | 7.47E−07 | 5.18E−06 | Up | Kidney | 0.947917 | 0.860465 | TRUE | 1 |
| ENST00000593491.2 | Kidney.Papillary.Cell.Carcinoma | 1.494278 | 1.498583 | −0.16692 | 2.06E−05 | 1.18E−04 | Up | Kidney | 0.795139 | 0.395349 | TRUE | 1 |
| ENST00000518073.1 | Liver.Hepatocellular.Carcinoma | 1.732605 | 1.735323 | 0.496941 | 9.72E−06 | 1.72E−04 | Up | Liver | 0.872629 | 0.7 | TRUE | 1 |
| ENST00000480284.1 | Lung.Adenocarcinoma | 5.102445 | 5.175009 | −0.00678 | 2.53E−35 | 3.88E−33 | Up | Lung | 0.717349 | 0 | TRUE | 1 |
| ENST00000608442.1 | Lung.Adenocarcinoma | 6.39745 | 6.401334 | 3.785682 | 5.73E−35 | 8.52E−33 | Up | Lung | 0.894737 | 0.495413 | TRUE | 1 |
| ENST00000578759.1 | Lung.Adenocarcinoma | 1.837704 | 1.841528 | 0.184133 | 3.11E−08 | 3.59E−07 | Up | Lung | 0.719298 | 0.577982 | TRUE | 1 |
| ENST00000500853.1 | Lung.Adenocarcinoma | 1.332907 | 1.3343 | 1.241197 | 8.24E−05 | 5.86E−04 | Up | Lung | 0.992203 | 1 | TRUE | 1 |
| ENST00000536835.2 | Lung.Adenocarcinoma | 1.620184 | 1.622618 | 1.105765 | 1.35E−04 | 9.23E−04 | Up | Lung | 0.826511 | 0.862385 | TRUE | 1 |
| ENST00000426615.3 | Lung.Squamous.Cell.Carcinoma | 4.692553 | 4.724449 | 0.154975 | 2.01E−54 | 2.34E−52 | Up | Lung | 0.795181 | 0 | TRUE | 1 |
| ENST00000335142.5 | Lung.Squamous.Cell.Carcinoma | 3.10978 | 3.130682 | −0.59886 | 3.86E−40 | 2.12E−38 | Up | Lung | 0.74498 | 0.009174 | TRUE | 1 |
| ENST00000602367.1 | Lung.Squamous.Cell.Carcinoma | 2.939312 | 2.94034 | 2.82483 | 2.21E−36 | 9.72E−35 | Up | Lung | 0.995984 | 0.990826 | TRUE | 1 |
| ENST00000438290.2 | Lung.Squamous.Cell.Carcinoma | 4.000009 | 4.006314 | 0.798526 | 8.47E−24 | 1.79E−22 | Up | Lung | 0.875502 | 0.293578 | TRUE | 1 |
| ENST00000602579.1 | Lung.Squamous.Cell.Carcinoma | 4.120598 | 4.128628 | 1.129311 | 3.97E−21 | 7.13E−20 | Up | Lung | 0.783133 | 0.174312 | TRUE | 1 |
| ENST00000521369.2 | Lung.Squamous.Cell.Carcinoma | 2.430787 | 2.435166 | 1.241594 | 6.47E−19 | 1.00E−17 | Up | Lung | 0.931727 | 0.605505 | TRUE | 1 |
| ENST00000508973.5 | Lung.Squamous.Cell.Carcinoma | 1.698774 | 1.700541 | 0.89685 | 8.67E−17 | 1.16E−15 | Up | Lung | 0.997992 | 1 | TRUE | 1 |
| ENST00000608756.5 | Lung.Squamous.Cell.Carcinoma | 1.397704 | 1.398206 | 2.74038 | 2.78E−09 | 2.11E−08 | Up | Lung | 1 | 1 | TRUE | 1 |
| ENST00000599421.1 | Lung.Squamous.Cell.Carcinoma | 2.104998 | 2.115153 | −0.55698 | 1.65E−08 | 1.17E−07 | Up | Lung | 0.759036 | 0.100917 | TRUE | 1 |
| ENST00000425081.2 | Lung.Squamous.Cell.Carcinoma | 1.452344 | 1.453427 | 1.953302 | 2.47E−08 | 1.72E−07 | Up | Lung | 1 | 0.990826 | TRUE | 1 |
| ENST00000412224.6 | Lung.Squamous.Cell.Carcinoma | 1.436948 | 1.439627 | 0.672832 | 1.13E−07 | 7.39E−07 | Up | Lung | 0.98996 | 0.807339 | TRUE | 1 |
| ENST00000439199.1 | Lung.Squamous.Cell.Carcinoma | 1.640485 | 1.644196 | −0.33051 | 1.86E−06 | 1.07E−05 | Up | Lung | 0.712851 | 0.577982 | TRUE | 1 |
| ENST00000342584.3 | Lung.Squamous.Cell.Carcinoma | 1.408359 | 1.408577 | 4.682606 | 6.90E−06 | 3.71E−05 | Up | Lung | 1 | 1 | TRUE | 1 |
| ENST00000609497.5 | Prostate.Adenocarcinoma | 1.7669 | 1.768577 | 0.797076 | 5.04E−07 | 2.76E−05 | Up | Prostate | 0.995951 | 0.941176 | TRUE | 1 |
| ENST00000548416.1 | Prostate.Adenocarcinoma | 2.873145 | 2.87548 | −0.27316 | 7.44E−06 | 2.89E−04 | Up | Prostate | 0.809717 | 0.352941 | TRUE | 1 |
| ENST00000562172.2 | Thyroid.Carcinoma | 2.201206 | 2.210978 | −0.75177 | 1.69E−08 | 8.56E−07 | Up | Thyroid | 0.876984 | 0.101695 | TRUE | 1 |
| | | | | | | | | Gland | | | | |
| ENST00000565118.1 | Thyroid.Carcinoma | 1.945381 | 1.94987 | 0.384857 | 1.92E−05 | 5.03E−04 | Up | Thyroid | 0.944444 | 0.440678 | TRUE | 1 |
| | | | | | | | | Gland | | | | |

TABLE 5

| ID | transcript | disease | tags.logFC | tags.unshrunk.logFC | tags.logCPM | tags.PValue | tags.FDR | decidetest |
|---|---|---|---|---|---|---|---|---|
| 9 | ENST00000540175.1 | Breast.Invasive.Carcinoma | 3.298631 | 3.315058 | 0.387586 | 3.24E−37 | 2.53E−35 | Up |
| 10 | ENST00000452320.3 | Breast.Invasive.Carcinoma | −2.19826 | −2.1986 | 3.433335 | 3.18E−33 | 2.14E−31 | Down |
| 22 | ENST00000548760.2 | Breast.Invasive.Carcinoma | 1.815116 | 1.81777 | 1.520554 | 2.28E−20 | 7.55E−19 | Up |
| 31 | ENST00000443294.5 | Breast.Invasive.Carcinoma | 2.480298 | 2.489582 | 0.297901 | 2.76E−17 | 7.34E−16 | Up |
| 39 | ENST00000574306.1 | Breast.Invasive.Carcinoma | −1.68776 | −1.68803 | 3.669806 | 5.43E−16 | 1.31E−14 | Down |
| 49 | ENST00000534398.1 | Breast.Invasive.Carcinoma | 2.043919 | 2.048391 | 0.778271 | 2.65E−13 | 5.07E−12 | Up |
| 52 | ENST00000608395.1 | Breast.Invasive.Carcinoma | −1.75765 | −1.7583 | 2.267482 | 6.51E−13 | 1.21E−11 | Down |
| 58 | ENST00000416221.5 | Breast.Invasive.Carcinoma | 1.765284 | 1.767079 | 2.000384 | 4.81E−11 | 7.47E−10 | Up |
| 81 | ENST00000562107.1 | Breast.Invasive.Carcinoma | 1.888175 | 1.889231 | 3.463399 | 2.99E−08 | 3.45E−07 | Up |
| 87 | ENST00000597156.1 | Breast.Invasive.Carcinoma | 2.054481 | 2.056969 | 0.487244 | 2.06E−07 | 2.14E−06 | Up |
| 92 | ENST00000527620.5 | Breast.Invasive.Carcinoma | 1.753555 | 1.75567 | 1.543816 | 1.02E−06 | 9.73E−06 | Up |
| 113 | ENST00000414457.5 | Breast.Invasive.Carcinoma | −1.30848 | −1.30977 | 0.386933 | 2.60E−05 | 2.03E−04 | Down |
| 128 | ENST00000559008.2 | Breast.Invasive.Carcinoma | 1.665734 | 1.66699 | 2.037803 | 1.16E−04 | 8.15E−04 | Up |
| 130 | ENST00000452962.1 | Breast.Invasive.Carcinoma | 1.312636 | 1.314646 | 0.227388 | 1.43E−04 | 9.92E−04 | Up |
| 897 | ENST00000342584.3 | Colon.Adenocarcinoma | −3.36685 | −3.36693 | 4.682606 | 9.34E−72 | 2.22E−68 | Down |
| 903 | ENST00000608395.1 | Colon.Adenocarcinoma | −2.53725 | −2.53791 | 2.267482 | 3.67E−19 | 5.38E−17 | Down |
| 909 | ENST00000531363.1 | Colon.Adenocarcinoma | 5.320282 | 5.414151 | −0.26462 | 1.72E−16 | 1.82E−14 | Up |
| 915 | ENST00000500112.1 | Colon.Adenocarcinoma | 4.640765 | 4.648384 | 1.195102 | 3.03E−12 | 1.74E−10 | Up |
| 921 | ENST00000534398.1 | Colon.Adenocarcinoma | 2.645632 | 2.654159 | 0.778271 | 4.92E−11 | 2.31E−09 | Up |
| 933 | ENST00000562298.1 | Colon.Adenocarcinoma | 3.000783 | 3.007491 | 0.790072 | 1.77E−09 | 6.10E−08 | Up |
| 935 | ENST00000572856.1 | Colon.Adenocarcinoma | 2.224274 | 2.226051 | 0.816254 | 4.27E−09 | 1.36E−07 | Up |
| 939 | ENST00000411824.1 | Colon.Adenocarcinoma | 3.895436 | 3.905732 | −1.01074 | 9.22E−09 | 2.74E−07 | Up |
| 941 | ENST00000574306.1 | Colon.Adenocarcinoma | −1.77407 | −1.77437 | 3.669806 | 1.63E−08 | 4.62E−07 | Down |
| 945 | ENST00000420367.1 | Colon.Adenocarcinoma | −1.63393 | −1.63614 | 0.582774 | 4.29E−08 | 1.11E−06 | Down |
| 918 | ENST00000545920.1 | Colon.Adenocarcinoma | 1.967722 | 1.975553 | −0.57706 | 2.19E−07 | 4.86E−06 | Up |
| 949 | ENST00000447221.1 | Colon.Adenocarcinoma | 1.733451 | 1.735968 | 0.802227 | 7.20E−07 | 1.42E−05 | Up |
| 959 | ENST00000417721.5 | Colon.Adenocarcinoma | 1.595769 | 1.596616 | 2.200341 | 4.80E−06 | 7.77E−05 | Up |
| 972 | ENST00000452079.5 | Colon.Adenocarcinoma | −1.49826 | −1.49913 | 3.602258 | 2.87E−05 | 3.78E−04 | Down |
| 976 | ENST00000449500.1 | Colon.Adenocarcinoma | 1.590162 | 1.59219 | 1.335273 | 5.76E−05 | 6.99E−04 | Up |
| 1804 | ENST00000608395.1 | Esophageal.Carcinoma | −2.36514 | −2.36562 | 2.267482 | 4.87E−07 | 4.70E−05 | Down |
| 2707 | ENST00000455557.2 | Head...Neck.Squamous.Cell.Carcinoma | 3.795759 | 3.80235 | 0.320549 | 8.36E−12 | 3.36E−10 | Up |
| 2710 | ENST00000629441.1 | Head...Neck.Squamous.Cell.Carcinoma | 2.046502 | 2.050326 | 1.080903 | 4.74E−10 | 1.54E−08 | Up |
| 2717 | ENST00000440326.1 | Head...Neck.Squamous.Cell.Carcinoma | 2.889075 | 2.897726 | −1.00815 | 1.20E−08 | 3.22E−07 | Up |
| 2730 | ENST00000572856.1 | Head...Neck.Squamous.Cell.Carcinoma | 1.884161 | 1.889108 | 0.816254 | 1.88E−06 | 3.47E−05 | Up |
| 2743 | ENST00000555918.1 | Head...Neck.Squamous.Cell.Carcinoma | 1.597474 | 1.603062 | −1.04289 | 1.21E−05 | 1.90E−04 | Up |
| 3586 | ENST00000454935.1 | Kidney.Chromophobe | 2.974637 | 2.975434 | 2.671972 | 7.04E−54 | 2.46E−51 | Up |
| 3590 | ENST00000449248.1 | Kidney.Chromophobe | 3.218519 | 3.221192 | 0.420296 | 8.56E−43 | 1.69E−40 | Up |
| 3594 | ENST00000342584.3 | Kidney.Chromophobe | −2.77421 | −2.77431 | 4.682606 | 1.01E−30 | 9.36E−29 | Down |
| 3598 | ENST00000412483.1 | Kidney.Chromophobe | 4.259647 | 4.282639 | −2.87174 | 2.49E−22 | 1.26E−20 | Up |
| 3608 | ENST00000555562.1 | Kidney.Chromophobe | 2.166804 | 2.168153 | 1.192743 | 6.70E−18 | 2.34E−16 | Up |
| 3662 | ENST00000562107.1 | Kidney.Chromophobe | −2.18322 | −2.18418 | 3.463399 | 4.59E−07 | 4.83E−06 | Down |
| 3664 | ENST00000445184.1 | Kidney.Chromophobe | 1.695813 | 1.701129 | −0.4045 | 5.86E−07 | 6.08E−06 | Up |
| 3666 | ENST00000503051.1 | Kidney.Chromophobe | −1.4546 | −1.45622 | 0.460423 | 7.74E−07 | 7.87E−06 | Down |
| 3668 | ENST00000500741.2 | Kidney.Chromophobe | −1.3783 | −1.37871 | 3.256661 | 1.33E−06 | 1.30E−05 | Down |
| 3672 | ENST00000322209.3 | Kidney.Chromophobe | −1.82413 | −1.8248 | 1.49832 | 2.42E−06 | 2.27E−05 | Down |
| 3673 | ENST00000447668.2 | Kidney.Chromophobe | −1.52601 | −1.52734 | 1.226064 | 2.44E−06 | 2.29E−05 | Down |
| 3674 | ENST00000499732.2 | Kidney.Chromophobe | 1.791219 | 1.791239 | 7.203578 | 3.15E−06 | 2.90E−05 | Up |
| 3684 | ENST00000548760.2 | Kidney.Chromophobe | 1.493038 | 1.494515 | 1.520554 | 8.78E−06 | 7.52E−05 | Up |
| 3689 | ENST00000602367.1 | Kidney.Chromophobe | −1.73983 | −1.7411 | 2.82483 | 1.74E−05 | 1.41E−04 | Down |
| 3693 | ENST00000485974.1 | Kidney.Chromophobe | 1.502998 | 1.508426 | −0.2057 | 3.04E−05 | 2.36E−04 | Up |
| 3694 | ENST00000541196.2 | Kidney.Chromophobe | 1.642473 | 1.642562 | 5.092878 | 3.09E−05 | 2.40E−04 | Up |
| 3696 | ENST00000586421.1 | Kidney.Chromophobe | 1.753202 | 1.757574 | −1.33018 | 5.89E−05 | 4.35E−04 | Up |
| 3699 | ENST00000444583.6 | Kidney.Chromophobe | 1.638157 | 1.639968 | 1.10706 | 8.87E−05 | 6.34E−04 | Up |
| 4482 | ENST00000441184.1 | Kidney.Clear.Cell.Carcinoma | 9.154483 | 9.516906 | −1.16397 | 1.49E−111 | 6.56E−109 | Up |
| 4485 | ENST00000478818.1 | Kidney.Clear.Cell.Carcinoma | 5.149938 | 5.190719 | −1.10395 | 1.19E−95 | 3.74E−93 | Up |
| 4488 | ENST00000342584.3 | Kidney.Clear.Cell.Carcinoma | −2.60224 | −2.60233 | 4.682606 | 3.65E−79 | 8.14E−77 | Down |
| 4493 | ENST00000455405.6 | Kidney.Clear.Cell.Carcinoma | −3.47741 | −3.47788 | 2.12831 | 1.65E−57 | 2.12E−55 | Down |
| 4497 | ENST00000609153.1 | Kidney.Clear.Cell.Carcinoma | 2.413685 | 2.41867 | −0.49467 | 1.21E−43 | 9.71E−42 | Up |
| 4500 | ENST00000608794.1 | Kidney.Clear.Cell.Carcinoma | 2.97377 | 2.979017 | −0.55779 | 1.65E−39 | 1.13E−37 | Up |
| 4509 | ENST00000322209.3 | Kidney.Clear.Cell.Carcinoma | −2.25509 | −2.25609 | 1.49832 | 3.12E−29 | 1.31E−27 | Down |
| 4519 | ENST00000583271.5 | Kidney.Clear.Cell.Carcinoma | 1.839558 | 1.839912 | 4.134732 | 2.11E−24 | 6.67E−23 | Up |
| 4531 | ENST00000481651.1 | Kidney.Clear.Cell.Carcinoma | 2.025692 | 2.026281 | 2.37328 | 6.45E−19 | 1.41E−17 | Up |
| 4535 | ENST00000503051.1 | Kidney.Clear.Cell.Carcinoma | −1.53313 | −1.53489 | 0.460423 | 1.91E−18 | 4.02E−17 | Down |
| 4537 | ENST00000621948.4 | Kidney.Clear.Cell.Carcinoma | 2.386688 | 2.388649 | 2.340846 | 4.56E−18 | 9.37E−17 | Up |
| 4542 | ENST00000562107.1 | Kidney.Clear.Cell.Carcinoma | −2.18705 | −2.18801 | 3.463399 | 9.30E−17 | 1.73E−15 | Down |
| 4545 | ENST00000448869.1 | Kidney.Clear.Cell.Carcinoma | 2.56632 | 2.577901 | 0.362374 | 3.29E−15 | 5.41E−14 | Up |
| 4546 | ENST00000417112.1 | Kidney.Clear.Cell.Carcinoma | 3.381002 | 3.400392 | −0.31786 | 4.90E−15 | 7.94E−14 | Up |
| 4551 | ENST00000411998.1 | Kidney.Clear.Cell.Carcinoma | 1.545576 | 1.546247 | 2.538026 | 3.43E−13 | 4.73E−12 | Up |
| 4556 | ENST00000527620.5 | Kidney.Clear.Cell.Carcinoma | 2.159296 | 2.160787 | 1.543816 | 1.85E−12 | 2.39E−11 | Up |
| 4558 | ENST00000612598.1 | Kidney.Clear.Cell.Carcinoma | −1.56786 | −1.56908 | 2.49122 | 4.30E−12 | 5.36E−11 | Down |
| 4562 | ENST00000591360.1 | Kidney.Clear.Cell.Carcinoma | 2.604186 | 2.61243 | −1.8961 | 2.95E−11 | 3.39E−10 | Up |
| 4563 | ENST00000452320.3 | Kidney.Clear.Cell.Carcinoma | −1.6654 | −1.66552 | 3.433335 | 4.33E−11 | 4.90E−10 | Down |
| 4577 | ENST00000457107.5 | Kidney.Clear.Cell.Carcinoma | 2.310997 | 2.314322 | 0.077257 | 4.05E−10 | 4.13E−09 | Up |
| 4582 | ENST00000445427.1 | Kidney.Clear.Cell.Carcinoma | −1.83839 | −1.83963 | 0.738744 | 8.86E−10 | 8.72E−09 | Down |
| 4584 | ENST00000542466.2 | Kidney.Clear.Cell.Carcinoma | 1.579937 | 1.580647 | 3.147912 | 1.08E−09 | 1.05E−08 | Up |
| 4595 | ENST00000511543.1 | Kidney.Clear.Cell.Carcinoma | 1.626799 | 1.631377 | −0.45215 | 1.55E−08 | 1.34E−07 | Up |
| 4600 | ENST00000606878.1 | Kidney.Clear.Cell.Carcinoma | 1.647561 | 1.653536 | −1.24256 | 7.16E−08 | 5.72E−07 | Up |
| 4618 | ENST00000606319.1 | Kidney.Clear.Cell.Carcinoma | 1.66922 | 1.671661 | −0.10856 | 2.55E−06 | 1.69E−05 | Up |
| 4622 | ENST00000461007.5 | Kidney.Clear.Cell.Carcinoma | 1.434008 | 1.435758 | 1.346947 | 4.26E−06 | 2.74E−05 | Up |
| 4625 | ENST00000426200.1 | Kidney.Clear.Cell.Carcinoma | −1.50515 | −1.50607 | 1.375218 | 8.46E−06 | 5.23E−05 | Down |
| 4630 | ENST00000441399.2 | Kidney.Clear.Cell.Carcinoma | −1.30663 | −1.3079 | 0.47803 | 1.49E−05 | 8.91E−05 | Down |
| 4638 | ENST00000608651.1 | Kidney.Clear.Cell.Carcinoma | −1.44839 | −1.44962 | 0.409595 | 4.47E−05 | 2.49E−04 | Down |
| 4646 | ENST00000331944.10 | Kidney.Clear.Cell.Carcinoma | 1.35959 | 1.35987 | 3.593675 | 6.33E−05 | 3.45E−04 | Up |
| 4661 | ENST00000501079.5 | Kidney.Clear.Cell.Carcinoma | −1.22676 | −1.22696 | 3.187274 | 1.74E−04 | 8.81E−04 | Down |
| 5379 | ENST00000342584.3 | Kidney.Papillary.Cell.Carcinoma | −3.08653 | −3.08665 | 4.682606 | 2.79E−103 | 2.14E−100 | Down |
| 5381 | ENST00000503051.1 | Kidney.Papillary.Cell.Carcinoma | −1.94079 | −1.94343 | 0.460423 | 8.46E−44 | 1.03E−41 | Down |
| 5390 | ENST00000621948.4 | Kidney.Papillary.Cell.Carcinoma | 3.178187 | 3.180343 | 2.340846 | 9.26E−34 | 6.54E−32 | Up |
| 5394 | ENST00000485974.1 | Kidney.Papillary.Cell.Carcinoma | 2.224716 | 2.231307 | −0.2057 | 2.21E−31 | 1.33E−29 | Up |
| 5399 | ENST00000322209.3 | Kidney.Papillary.Cell.Carcinoma | −2.39518 | −2.39631 | 1.49832 | 6.43E−29 | 3.19E−27 | Down |
| 5400 | ENST00000455405.6 | Kidney.Papillary.Cell.Carcinoma | −2.98074 | −2.98106 | 2.12831 | 1.01E−28 | 4.96E−27 | Down |
| 5401 | ENST00000448869.1 | Kidney.Papillary.Cell.Carcinoma | 3.478525 | 3.491207 | 0.362374 | 3.44E−28 | 1.62E−26 | Up |
| 5409 | ENST00000417112.1 | Kidney.Papillary.Cell.Carcinoma | 4.071537 | 4.091701 | −0.31786 | 2.63E−20 | 6.77E−19 | Up |
| 5414 | ENST00000527620.5 | Kidney.Papillary.Cell.Carcinoma | 2.597729 | 2.599334 | 1.543816 | 1.29E−18 | 2.90E−17 | Up |
| 5415 | ENST00000428939.3 | Kidney.Papillary.Cell.Carcinoma | 2.470259 | 2.474072 | 0.0112 | 8.15E−18 | 1.70E−16 | Up |
| 5447 | ENST00000411998.1 | Kidney.Papillary.Cell.Carcinoma | 1.480662 | 1.481316 | 2.538026 | 7.07E−10 | 6.97E−09 | Up |
| 5454 | ENST00000583271.5 | Kidney.Papillary.Cell.Carcinoma | 1.47792 | 1.478235 | 4.134732 | 3.55E−09 | 3.24E−08 | Up |
| 5456 | ENST00000426200.1 | Kidney.Papillary.Cell.Carcinoma | −1.74791 | −1.7491 | 1.375218 | 8.00E−09 | 7.02E−08 | Down |
| 5467 | ENST00000592638.1 | Kidney.Papillary.Cell.Carcinoma | −1.54994 | −1.55153 | 0.164064 | 1.85E−07 | 1.39E−06 | Down |
| 5473 | ENST00000429630.1 | Kidney.Papillary.Cell.Carcinoma | 1.434365 | 1.434969 | 1.717205 | 3.85E−07 | 2.77E−06 | Up |
| 5478 | ENST00000568654.1 | Kidney.Papillary.Cell.Carcinoma | 1.624048 | 1.626416 | 0.937888 | 7.47E−07 | 5.18E−06 | Up |
| 5482 | ENST00000444583.6 | Kidney.Papillary.Cell.Carcinoma | 1.614463 | 1.61626 | 1.10706 | 1.20E−06 | 8.11E−06 | Up |
| 5484 | ENST00000449248.1 | Kidney.Papillary.Cell.Carcinoma | 1.516384 | 1.518332 | 0.420296 | 1.67E−06 | 1.11E−05 | Up |
| 5490 | ENST00000461007.5 | Kidney.Papillary.Cell.Carcinoma | 1.470691 | 1.472467 | 1.346947 | 3.32E−06 | 2.12E−05 | Up |
| 5493 | ENST00000457107.5 | Kidney.Papillary.Cell.Carcinoma | 1.923672 | 1.926739 | 0.077257 | 5.44E−06 | 3.37E−05 | Up |
| 5501 | ENST00000606878.1 | Kidney.Papillary.Cell.Carcinoma | 1.528552 | 1.534287 | −1.24256 | 1.30E−05 | 7.64E−05 | Up |
| 5503 | ENST00000593491.2 | Kidney.Papillary.Cell.Carcinoma | 1.494278 | 1.498583 | −0.16692 | 2.06E−05 | 1.18E−04 | Up |
| 5507 | ENST00000606319.1 | Kidney.Papillary.Cell.Carcinoma | 1.613912 | 1.616309 | −0.10856 | 2.76E−05 | 1.55E−04 | Up |
| 5511 | ENST00000421685.2 | Kidney.Papillary.Cell.Carcinoma | −1.2452 | −1.24602 | 1.622244 | 5.69E−05 | 3.05E−04 | Down |
| 5532 | ENST00000417194.5 | Kidney.Papillary.Cell.Carcinoma | 1.339807 | 1.340727 | 1.178262 | 1.92E−04 | 9.44E−04 | Up |
| 6294 | ENST00000416221.5 | Liver.Hepatocellular.Carcinoma | 2.248226 | 2.249063 | 2.000384 | 1.54E−11 | 9.83E−10 | Up |
| 6304 | ENST00000452320.3 | Liver.Hepatocellular.Carcinoma | −1.90822 | −1.90847 | 3.433335 | 9.27E−10 | 4.17E−08 | Down |
| 6313 | ENST00000331944.10 | Liver.Hepatocellular.Carcinoma | 1.923082 | 1.923501 | 3.593675 | 5.61E−09 | 2.13E−07 | Up |
| 6314 | ENST00000443294.5 | Liver.Hepatocellular.Carcinoma | 2.46167 | 2.468868 | 0.297901 | 1.11E−08 | 3.93E−07 | Up |
| 6322 | ENST00000447221.1 | Liver.Hepatocellular.Carcinoma | 1.770286 | 1.774956 | 0.802227 | 2.63E−08 | 8.51E−07 | Up |
| 6331 | ENST00000621948.4 | Liver.Hepatocellular.Carcinoma | 2.234873 | 2.238024 | 2.340846 | 1.35E−07 | 3.76E−06 | Up |
| 6334 | ENST00000417194.5 | Liver.Hepatocellular.Carcinoma | 1.771586 | 1.776189 | 1.178262 | 2.81E−07 | 7.29E−06 | Up |
| 6337 | ENST00000548760.2 | Liver.Hepatocellular.Carcinoma | 1.598293 | 1.599529 | 1.520554 | 8.68E−07 | 2.02E−05 | Up |
| 6312 | ENST00000485974.1 | Liver.Hepatocellular.Carcinoma | 1.626669 | 1.631876 | −0.2057 | 3.26E−06 | 6.57E−05 | Up |
| 6345 | ENST00000518073.1 | Liver.Hepatocellular.Carcinoma | 1.732605 | 1.735323 | 0.496941 | 9.72E−06 | 1.72E−04 | Up |
| 7175 | ENST00000416221.5 | Lung.Adenocarcinoma | 2.751616 | 2.754891 | 2.000384 | 4.89E−36 | 7.96E−34 | Up |
| 7176 | ENST00000480284.1 | Lung.Adenocarcinoma | 5.102445 | 5.175009 | −0.00678 | 2.53E−35 | 3.88E−33 | Up |
| 7178 | ENST00000608442.1 | Lung.Adenocarcinoma | 6.39745 | 6.401334 | 3.785682 | 5.73E−35 | 8.52E−33 | Up |
| 7183 | ENST00000540175.1 | Lung.Adenocarcinoma | 3.027936 | 3.045455 | 0.387586 | 1.41E−28 | 1.21E−26 | Up |
| 7203 | ENST00000608395.1 | Lung.Adenocarcinoma | −2.05156 | −2.0524 | 2.267482 | 4.91E−20 | 1.88E−18 | Down |
| 7212 | ENST00000619960.4 | Lung.Adenocarcinoma | 2.194658 | 2.200117 | 0.525588 | 7.64E−17 | 2.18E−15 | Up |
| 7213 | ENST00000574306.1 | Lung.Adenocarcinoma | −1.76397 | −1.76415 | 3.669806 | 8.13E−17 | 2.31E−15 | Down |
| 7220 | ENST00000443294.5 | Lung.Adenocarcinoma | 2.483636 | 2.493804 | 0.297901 | 5.54E−16 | 1.46E−14 | Up |
| 7226 | ENST00000398832.2 | Lung.Adenocarcinoma | −2.00168 | −2.003 | −0.24237 | 5.85E.15 | 1.39E−13 | Down |
| 7236 | ENST00000452320.3 | Lung.Adenocarcinoma | −1.80194 | −1.8022 | 3.433335 | 1.44E−13 | 2.96E−12 | Down |
| 7239 | ENST00000534398.1 | Lung.Adenocarcinoma | 2.10339 | 2.109801 | 0.778271 | 2.97E−13 | 5.90E−12 | Up |
| 7249 | ENST00000612598.1 | Lung.Adenocarcinoma | 1.642524 | 1.644166 | 2.49122 | 2.17E−11 | 3.57E−10 | Up |
| 7258 | ENST00000452962.1 | Lung.Adenocarcinoma | 1.613782 | 1.619177 | 0.227388 | 7.45E−10 | 1.04E−08 | Up |
| 7269 | ENST00000414457.5 | Lung.Adenocarcinoma | −1.4733 | −1.47517 | 0.386933 | 1.22E−08 | 1.47E−07 | Down |
| 7274 | ENST00000548760.2 | Lung.Adenocarcinoma | 1.477624 | 1.480022 | 1.520554 | 2.33E−08 | 2.73E−07 | Up |
| 7275 | ENST00000578759.1 | Lung.Adenocarcinoma | 1.837704 | 1.841528 | 0.184133 | 3.11E−08 | 3.59E−07 | Up |
| 7276 | ENST00000486431.5 | Lung.Adenocarcinoma | 2.146846 | 2.153605 | 0.537806 | 3.95E−08 | 4.50E−07 | Up |
| 7298 | ENST00000625168.1 | Lung.Adenocarcinoma | −1.32241 | −1.32336 | 1.160636 | 4.94E−06 | 4.27E−05 | Down |
| 7300 | ENST00000597156.1 | Lung.Adenocarcinoma | 1.909107 | 1.913501 | 0.487244 | 8.82E−06 | 7.31E−05 | Up |
| 7316 | ENST00000466734.5 | Lung.Adenocarcinoma | −1.34872 | −1.34956 | 1.843548 | 7.07E−05 | 5.08E−04 | Down |
| 7318 | ENST00000500853.1 | Lung.Adenocarcinoma | 1.332907 | 1.3343 | 1.241197 | 8.24E−05 | 5.86E−04 | Up |
| 7321 | ENST00000536835.2 | Lung.Adenocarcinoma | 1.620184 | 1.622618 | 1.105765 | 1.35E−04 | 9.23E−04 | Up |
| 8069 | ENST00000531363.1 | Lung.Squamous.Cell.Carcinoma | 7.543336 | 7.786061 | −0.26462 | 9.36E−64 | 1.61E−61 | Up |
| 8070 | ENST00000426615.3 | Lung.Squamous.Cell.Carcinoma | 4.692553 | 4.724449 | 0.154975 | 2.01E−54 | 2.34E−52 | Up |
| 8073 | ENST00000540175.1 | Lung.Squamous.Cell.Carcinoma | 3.886351 | 3.904969 | 0.387586 | 2.31E−48 | 2.02E−46 | Up |
| 8075 | ENST00000323813.3 | Lung.Squamous.Cell.Carcinoma | 3.216598 | 3.239229 | 0.358171 | 1.56E−45 | 1.17E−43 | Up |
| 8076 | ENST00000416221.5 | Lung.Squamous.Cell.Carcinoma | 2.978124 | 2.981481 | 2.000384 | 1.72E−43 | 1.14E−41 | Up |
| 8077 | ENST00000548760.2 | Lung.Squamous.Cell.Carcinoma | 2.377398 | 2.380419 | 1.520554 | 1.82E−43 | 1.21E−41 | Up |
| 8082 | ENST00000335142.5 | Lung.Squamous.Cell.Carcinoma | 3.10978 | 3.130682 | −0.59886 | 3.86E−40 | 2.12E−38 | Up |
| 8083 | ENST00000562298.1 | Lung.Squamous.Cell.Carcinoma | 4.207956 | 4.230156 | 0.790072 | 8.34E−40 | 4.49E−38 | Up |
| 8087 | ENST00000452320.3 | Lung.Squamous.Cell.Carcinoma | −2.38496 | −2.38541 | 3.433335 | 7.13E−37 | 3.24E−35 | Down |
| 8090 | ENST00000602367.1 | Lung.Squamous.Cell.Carcinoma | 2.939312 | 2.94034 | 2.82483 | 2.21E−36 | 9.72E−35 | Up |
| 8092 | ENST00000612598.1 | Lung.Squamous.Cell.Carcinoma | 2.298197 | 2.300122 | 2.49122 | 2.29E−34 | 8.95E−33 | Up |
| 8093 | ENST00000534398.1 | Lung.Squamous.Cell.Carcinoma | 3.044935 | 3.052275 | 0.778271 | 1.07E−33 | 4.03E−32 | Up |
| 8110 | ENST00000574306.1 | Lung.Squamous.Cell.Carcinoma | −1.96365 | −1.96386 | 3.669806 | 1.77E−25 | 4.14E−24 | Down |
| 8111 | ENST00000619960.4 | Lung.Squamous.Cell.Carcinoma | 2.556172 | 2.561969 | 0.525588 | 3.20E−25 | 7.36E−24 | Up |
| 8116 | ENST00000545920.1 | Lung.Squamous.Cell.Carcinoma | 2.304102 | 2.313743 | −0.57706 | 8.21E−24 | 1.74E−22 | Up |
| 8117 | ENST00000438290.2 | Lung.Squamous.Cell.Carcinoma | 4.000009 | 4.006314 | 0.798526 | 8.47E−24 | 1.79E−22 | Up |
| 8124 | ENST00000486431.5 | Lung.Squamous.Cell.Carcinoma | 3.350835 | 3.358705 | 0.537806 | 5.14E−22 | 9.77E−21 | Up |
| 8130 | ENST00000602579.1 | Lung.Squamous.Cell.Carcinoma | 4.120598 | 4.128628 | 1.129311 | 3.97E−21 | 7.13E−20 | Up |
| 8140 | ENST00000521369.2 | Lung.Squamous.Cell.Carcinoma | 2.430787 | 2.435166 | 1.241594 | 6.47E−19 | 1.00E−17 | Up |
| 8151 | ENST00000508973.5 | Lung.Squamous.Cell.Carcinoma | 1.698774 | 1.700541 | 0.89685 | 8.67E−17 | 1.16E−15 | Up |
| 8176 | ENST00000577066.2 | Lung.Squamous.Cell.Carcinoma | −1.48973 | −1.48986 | 4.695719 | 3.44E−13 | 3.59E−12 | Down |
| 8178 | ENST00000562107.1 | Lung.Squamous.Cell.Carcinoma | 2.309017 | 2.311413 | 3.463399 | 5.45E−13 | 5.61E−12 | Up |
| 8184 | ENST00000625168.1 | Lung.Squamous.Cell.Carcinoma | −1.52693 | −1.52811 | 1.160636 | 2.11E−12 | 2.06E−11 | Down |
| 8211 | ENST00000608756.5 | Lung.Squamous.Cell.Carcinoma | 1.397704 | 1.398206 | 2.74038 | 2.78E−09 | 2.11E−08 | Up |
| 8218 | ENST00000599421.1 | Lung.Squamous.Cell.Carcinoma | 2.104998 | 2.115153 | −0.55698 | 1.65E−08 | 1.17E−07 | Up |
| 8222 | ENST00000425081.2 | Lung.Squamous.Cell.Carcinoma | 1.452344 | 1.453427 | 1.953302 | 2.47E−08 | 1.72E−07 | Up |
| 8227 | ENST00000572856.1 | Lung.Squamous.Cell.Carcinoma | 1.673982 | 1.675729 | 0.816254 | 4.84E−08 | 3.27E−07 | Up |
| 8229 | ENST00000412224.6 | Lung.Squamous.Cell.Carcinoma | 1.436948 | 1.439627 | 0.672832 | 1.13E−07 | 7.39E−07 | Up |
| 8238 | ENST00000621948.4 | Lung.Squamous.Cell.Carcinoma | 1.7732 | 1.774063 | 2.340846 | 4.93E−07 | 3.02E−06 | Up |
| 8242 | ENST00000542466.2 | Lung.Squamous.Cell.Carcinoma | 1.489021 | 1.489388 | 3.147912 | 6.74E−07 | 4.06E−06 | Up |
| 8251 | ENST00000439199.1 | Lung.Squamous.Cell.Carcinoma | 1.640485 | 1.644196 | −0.33051 | 1.86E−06 | 1.07E−05 | Up |
| 8257 | ENST00000342584.3 | Lung.Squamous.Cell.Carcinoma | 1.408359 | 1.408577 | 4.682606 | 6.90E−06 | 3.71E−05 | Up |
| 8270 | ENST00000452962.1 | Lung.Squamous.Cell.Carcinoma | 1.376299 | 1.381225 | 0.227388 | 3.18E−05 | 1.58E−04 | Up |
| 8977 | ENST00000609497.5 | Prostate.Adenocarcinoma | 1.7669 | 1.768577 | 0.797076 | 5.04E−07 | 2.76E−05 | Up |
| 8984 | ENST00000548416.1 | Prostate.Adenocarcinoma | 2.873145 | 2.87548 | −0.27316 | 7.44E−06 | 2.89E−04 | Up |
| 8992 | ENST00000562298.1 | Prostate.Adenocarcinoma | 2.050948 | 2.053316 | 0.790072 | 3.05E−05 | 9.72E−04 | Up |
| 9862 | ENST00000572856.1 | Stomach.Adenocarcinoma | 2.593733 | 2.59941 | 0.816254 | 6.14E−12 | 4.49E−10 | Up |
| 9892 | ENST00000608395.1 | Stomach.Adenocarcinoma | −1.74175 | −1.742 | 2.267482 | 1.29E−05 | 2.36E−04 | Down |
| 10760 | ENST00000597156.1 | Thyroid.Carcinoma | 3.314797 | 3.328631 | 0.487244 | 1.98E−12 | 1.96E−10 | Up |
| 10767 | ENST00000562172.2 | Thyroid.Carcinoma | 2.201206 | 2.210978 | −0.75177 | 1.69E−08 | 8.56E−07 | Up |
| 10783 | ENST00000565118.1 | Thyroid.Carcinoma | 1.945381 | 1.94987 | 0.384857 | 1.92E−05 | 5.03E−04 | Up |
| 11650 | ENST00000608395.1 | Uterine.Corpus.Endometrioid.Carcinoma | −3.78498 | −3.7861 | 2.267482 | 2.13E−39 | 2.81E−36 | Down |
| 11652 | ENST00000452320.3 | Uterine.Corpus.Endometrioid.Carcinoma | −3.39304 | −3.39398 | 3.433335 | 1.80E−31 | 1.15E−28 | Down |
| 11662 | ENST00000562172.2 | Uterine.Corpus.Endometrioid.Carcinoma | −2.82404 | −2.82593 | −0.75177 | 1.23E−11 | 8.31E−10 | Down |
| 11664 | ENST00000437764.5 | Uterine.Corpus.Endometrioid.Carcinoma | −2.45985 | −2.46156 | 0.628165 | 3.72E−10 | 1.94E−08 | Down |
| 11674 | ENST00000416221.5 | Uterine.Corpus.Endometrioid.Carcinoma | 2.551993 | 2.554599 | 2.000384 | 1.29E−08 | 4.76E−07 | Up |
| 11679 | ENST00000443294.5 | Uterine.Corpus.Endometrioid.Carcinoma | 3.221104 | 3.233286 | 0.297901 | 2.07E−08 | 7.20E−07 | Up |
| 11685 | ENST00000323813.3 | Uterine.Corpus.Endometrioid.Carcinoma | 2.486297 | 2.494495 | 0.358171 | 1.80E−07 | 4.93E−06 | Up |
| 11714 | ENST00000452962.1 | Uterine.Corpus.Endometrioid.Carcinoma | 1.897415 | 1.905351 | 0.227388 | 1.03E−05 | 1.64E−04 | Up |

| | | prop_ex- | prop_ex- | is.freq.ex- | surviv- | surviv- | surviv- | surviv- | |
|---|---|---|---|---|---|---|---|---|---|
| ID | tissue | pressed_disease | pressed_tissue | pressed | al_km_pval_fdr | al_wald_pval_fdr | al_wald_test | al_hr | sig_class |
| 9 | Breast | 0.813187 | 0.097345 | TRUE | 0.190633 | 0.193434 | 2.24 | 0.773594 | NA |
| 10 | Breast | 0.995421 | 1 | TRUE | 0.3193 | 0.321885 | 1.12 | 1.196597 | NA |
| 22 | Breast | 0.997253 | 0.902655 | TRUE | 0.182659 | 0.185078 | 2.35 | 0.765553 | NA |
| 31 | Breast | 0.717949 | 0.123894 | TRUE | 0.003076 | 0.00375 | 12.75 | 0.554389 | 0.001 |
| | | | | | | | | | a ‰¤ |
| | | | | | | | | | p < 0.01 |
| 39 | Breast | 0.998168 | 1 | TRUE | 0.031104 | 0.035143 | 7.22 | 0.622799 | 0.01 |
| | | | | | | | | | a ‰¤ |
| | | | | | | | | | p < 0.05 |
| 49 | Breast | 0.90293 | 0.513274 | TRUE | 0.003366 | 0.004171 | 12.27 | 0.551971 | 0.001 |
| | | | | | | | | | a ‰¤ |
| | | | | | | | | | p < 0.01 |
| 52 | Breast | 0.990842 | 1 | TRUE | 0.038159 | 0.04135 | 6.75 | 1.534266 | 0.01 |
| | | | | | | | | | a ‰¤ |
| | | | | | | | | | p < 0.05 |
| 58 | Breast | 0.991758 | 0.99115 | TRUE | 0.335656 | 0.339434 | 1.02 | 1.179974 | NA |
| 81 | Breast | 0.931319 | 0.929204 | TRUE | 0.170726 | 0.177932 | 2.48 | 0.756882 | NA |
| 87 | Breast | 0.894689 | 0.761062 | TRUE | 0.1521 | 0.159597 | 2.82 | 0.759903 | NA |
| 92 | Breast | 0.821429 | 0.646018 | TRUE | 0.027349 | 0.032118 | 7.48 | 1.638274 | 0.01 |
| | | | | | | | | | a ‰¤ |
| | | | | | | | | | p < 0.05 |
| 113 | Breast | 0.92674 | 1 | TRUE | 0.394644 | 0.397741 | 0.76 | 0.864457 | NA |
| 128 | Breast | 0.880952 | 0.823009 | TRUE | 0.3193 | 0.321885 | 1.13 | 0.837471 | NA |
| 130 | Breast | 0.968864 | 0.867257 | TRUE | 0.1552 | 0.162831 | 2.68 | 1.336138 | NA |
| 897 | Colon | 1 | 1 | TRUE | 0.080855 | 0.091987 | 4.52 | 1.824542 | NA |
| 903 | Colon | 0.996516 | 1 | TRUE | 0.228517 | 0.232631 | 1.84 | 1.387289 | NA |
| 909 | Colon | 0.735192 | 0 | TRUE | 0.039465 | 0.04352 | 6.55 | 0.53977 | 0.01 |
| | | | | | | | | | a ‰¤ |
| | | | | | | | | | p < 0.05 |
| 915 | Colon | 0.972125 | 0.195122 | TRUE | 0.302782 | 0.306228 | 1.23 | 1.308857 | NA |
| 921 | Colon | 0.926829 | 0.097561 | TRUE | 0.289507 | 0.29483 | 1.32 | 1.319406 | NA |
| 933 | Colon | 0.930314 | 0.317073 | TRUE | 0.264891 | 0.270575 | 1.55 | 1.356806 | NA |
| 935 | Colon | 0.996516 | 0.95122 | TRUE | 0.369794 | 0.372976 | 0.85 | 0.79124 | NA |
| 939 | Colon | 0.783972 | 0.04878 | TRUE | 0.073053 | 0.079457 | 4.93 | 0.584975 | NA |
| 941 | Colon | 1 | 1 | TRUE | 0.128241 | 0.135218 | 3.31 | 1.556683 | NA |
| 945 | Colon | 0.885017 | 1 | TRUE | 0.075614 | 0.081949 | 4.83 | 0.586484 | NA |
| 918 | Colon | 0.829268 | 0.097561 | TRUE | 0.095928 | 0.103313 | 4.12 | 0.605378 | NA |
| 949 | Colon | 0.986063 | 0.902439 | TRUE | 0.044765 | 0.051488 | 6.02 | 0.553184 | NA |
| 959 | Colon | 0.996516 | 1 | TRUE | 0.394644 | 0.397741 | 0.76 | 0.801669 | NA |
| 972 | Colon | 0.878049 | 1 | TRUE | 0.153837 | 0.159597 | 2.74 | 0.667245 | NA |
| 976 | Colon | 0.989547 | 0.97561 | TRUE | 0.031534 | 0.03859 | 6.96 | 0.477798 | 0.01 |
| | | | | | | | | | a ‰¤ |
| | | | | | | | | | p < 0.05 |
| 1804 | Esophagus | 0.994475 | 1 | TRUE | 0.45156 | 0.454358 | 0.58 | 0.83489 | NA |
| 2707 | Head and | 0.714286 | 0.090909 | TRUE | 0.15479 | 0.159597 | 2.74 | 1.254026 | NA |
| | Neck region | | | | | | | | |
| 2710 | Head and | 0.953668 | 0.386361 | TRUE | 0.040935 | 0.04352 | 6.54 | 0.696097 | 0.01 |
| | Neck region | | | | | | | | a ‰¤ |
| | | | | | | | | | p < 0.05 |
| 2717 | Head and | 0.797297 | 0.181818 | TRUE | 2.82E-04 | 3.73E-04 | 18.23 | 0.551766 | 0.0001 |
| | Neck region | | | | | | | | a ‰¤ |
| | | | | | | | | | p < 0.001 |
| 2730 | Head and | 0.805019 | 0.318182 | TRUE | 0.043569 | 0.046062 | 6.35 | 0.709811 | 0.01 |
| | Neck region | | | | | | | | a ‰¤ |
| | | | | | | | | | p < 0.05 |
| 2743 | Head and | 0.828185 | 0.204545 | TRUE | 0.080855 | 0.089933 | 4.59 | 0.736561 | NA |
| | Neck region | | | | | | | | |
| 3586 | Kidney | 1 | 1 | TRUE | 0.496552 | 0.503237 | 0.46 | 0.61933 | NA |
| 3590 | Kidney | 1 | 0.821705 | TRUE | 0.274121 | 0.287794 | 1.39 | 2.210566 | NA |
| 3594 | Kidney | 1 | 1 | TRUE | 0.135149 | 0.159597 | 2.76 | 0.263817 | NA |
| 3598 | Kidney | 0.742424 | 0.007752 | TRUE | 0.044263 | 0.998098 | 0 | 4.38E+08 | NA |
| 3608 | Kidney | 0.984848 | 1 | TRUE | 0.196752 | 0.21429 | 2.01 | 0.366984 | NA |
| 3662 | Kidney | 0.833333 | 0.984496 | TRUE | 0.327135 | 0.337322 | 1.04 | 0.505123 | NA |
| 3664 | Kidney | 0.80303 | 0.263566 | TRUE | 0.232761 | 0.255828 | 1.65 | 2.805102 | NA |
| 3666 | Kidney | 0.909091 | 1 | TRUE | 0.288655 | 0.306228 | 1.25 | 2.45366 | NA |
| 3668 | Kidney | 1 | 1 | TRUE | 0.04386 | 0.097583 | 4.29 | 0.111095 | NA |
| 3672 | Kidney | 0.727273 | 1 | TRUE | 0.28056 | 0.290807 | 1.34 | 0.458306 | NA |
| 3673 | Kidney | 0.924242 | 1 | TRUE | 0.496552 | 0.503237 | 0.45 | 1.610735 | NA |
| 3674 | Kidney | 1 | 1 | TRUE | 0.096837 | 0.12001 | 3.61 | 3.611385 | NA |
| 3684 | Kidney | 1 | 1 | TRUE | 0.011792 | 0.04135 | 6.78 | 0.12404 | 0.01 |
| | | | | | | | | | a ‰¤ |
| | | | | | | | | | p < 0.05 |
| 3689 | Kidney | 0.833333 | 1 | TRUE | 0.013342 | 0.057186 | 5.65 | 0.079968 | NA |
| 3693 | Kidney | 0.818182 | 0.224806 | TRUE | 0.328317 | 0.340442 | 1.01 | 0.446843 | NA |
| 3694 | Kidney | 1 | 1 | TRUE | 0.1552 | 0.185078 | 2.36 | 3.432537 | NA |
| 3696 | Kidney | 0.757576 | 0.503876 | TRUE | 0.14807 | 0.162831 | 2.66 | 3.170854 | NA |
| 3699 | Kidney | 0.878788 | 0.844961 | TRUE | 0.249221 | 0.270725 | 1.54 | 0.369681 | NA |
| 4482 | Kidney | 0.707547 | 0 | TRUE | 0.283599 | 0.287794 | 1.37 | 1.195 | NA |
| 4485 | Kidney | 0.877358 | 0.007752 | TRUE | 0.1521 | 0.159597 | 2.87 | 0.768561 | NA |
| 4488 | Kidney | 1 | 1 | TRUE | 0.110023 | 0.114256 | 3.71 | 1.343311 | NA |
| 4493 | Kidney | 0.764151 | 0.992248 | TRUE | 0.004982 | 0.0058 | 11.48 | 1.72742 | 0.001 |
| | | | | | | | | | a ‰¤ |
| | | | | | | | | | p < 0.01 |
| 4497 | Kidney | 0.943396 | 0.395349 | TRUE | 0.049687 | 0.055222 | 5.84 | 0.691667 | NA |
| 4500 | Kidney | 0.943396 | 0.434109 | TRUE | 0.274121 | 0.27864 | 1.46 | 1.202835 | NA |
| 4509 | Kidney | 0.898113 | 1 | TRUE | 1.60E-07 | 7.82E-07 | 34.57 | 2.771759 | p < 0.0001 |
| 4519 | Kidney | 0.996226 | 1 | TRUE | 9.58E-07 | 2.55E-06 | 30.23 | 0.415068 | p < 0.0001 |
| 4531 | Kidney | 0.996226 | 1 | TRUE | 0.144371 | 0.150077 | 3.03 | 1.314035 | NA |
| 4535 | Kidney | 0.956604 | 1 | TRUE | 2.15E-04 | 3.11E-04 | 18.77 | 1.952947 | 0.0001 |
| | | | | | | | | | a ‰¤ |
| | | | | | | | | | p < 0.001 |
| 4537 | Kidney | 0.928302 | 0.782946 | TRUE | 0.001323 | 0.001659 | 14.63 | 0.547805 | 0.001 |
| | | | | | | | | | a ‰¤ |
| | | | | | | | | | p < 0.01 |
| 4542 | Kidney | 0.843396 | 0.984496 | TRUE | 0.04386 | 0.047544 | 6.24 | 1.488138 | 0.01 |
| | | | | | | | | | a ‰¤ |
| | | | | | | | | | p < 0.05 |
| 4545 | Kidney | 0.830189 | 0.085271 | TRUE | 0.477122 | 0.479674 | 0.52 | 0.894661 | NA |
| 4546 | Kidney | 0.767925 | 0.031008 | TRUE | 0.10303 | 0.108268 | 3.9 | 1.363388 | NA |
| 4551 | Kidney | 0.998113 | 1 | TRUE | 9.58E-07 | 2.55E-06 | 30.14 | 0.421968 | p < 0.0001 |
| 4556 | Kidney | 0.896226 | 0.620155 | TRUE | 0.001316 | 0.00165 | 14.77 | 0.554291 | 0.001 |
| | | | | | | | | | a ‰¤ |
| | | | | | | | | | p < 0.01 |
| 4558 | Kidney | 0.983019 | 1 | TRUE | 0.011851 | 0.013446 | 9.38 | 0.627255 | 0.01 |
| | | | | | | | | | a ‰¤ |
| | | | | | | | | | p < 0.05 |
| 4562 | Kidney | 0.786792 | 0.217054 | TRUE | 0.075815 | 0.081949 | 4.84 | 0.703166 | NA |
| 4563 | Kidney | 1 | 1 | TRUE | 0.008645 | 0.009671 | 10.38 | 1.633688 | 0.001 |
| | | | | | | | | | a ‰¤ |
| | | | | | | | | | p < 0.01 |
| 4577 | Kidney | 0.913208 | 0.511628 | TRUE | 0.074046 | 0.079457 | 4.96 | 1.405145 | NA |
| 4582 | Kidney | 0.896226 | 1 | TRUE | 0.357398 | 0.359951 | 0.92 | 1.165786 | NA |
| 4584 | Kidney | 0.992453 | 1 | TRUE | 4.32E-05 | 7.04E-05 | 21.84 | 0.486517 | p < 0.0001 |
| 4595 | Kidney | 0.8 | 0.356589 | TRUE | 0.00997 | 0.011157 | 10.05 | 0.612031 | 0.01 |
| | | | | | | | | | a ‰¤ |
| | | | | | | | | | p < 0.05 |
| 4600 | Kidney | 0.771698 | 0.217054 | TRUE | 6.56E-04 | 8.70E-04 | 16.44 | 0.537772 | 0.0001 |
| | | | | | | | | | a ‰¤ |
| | | | | | | | | | p < 0.001 |
| 4618 | Kidney | 0.762264 | 0.736434 | TRUE | 0.10303 | 0.108268 | 3.89 | 1.359761 | NA |
| 4622 | Kidney | 0.915094 | 0.821705 | TRUE | 4.32E-05 | 7.04E-05 | 22 | 0.482088 | p < 0.0001 |
| 4625 | Kidney | 0.839623 | 1 | TRUE | 3.10E-06 | 6.60E-06 | 27.31 | 2.215993 | p < 0.0001 |
| 4630 | Kidney | 0.95283 | 1 | TRUE | 3.10E-06 | 6.60E-06 | 27.69 | 2.231074 | p < 0.0001 |
| 4638 | Kidney | 0.826415 | 1 | TRUE | 8.49E-04 | 0.001111 | 15.81 | 1.85021 | 0.001 |
| | | | | | | | | | a ‰¤ |
| | | | | | | | | | p < 0.01 |
| 4646 | Kidney | 1 | 1 | TRUE | 1.97E-05 | 3.49E-05 | 23.74 | 0.475867 | p < 0.0001 |
| 4661 | Kidney | 1 | 1 | TRUE | 0.00997 | 0.011306 | 9.95 | 1.689497 | 0.01 |
| | | | | | | | | | a ‰¤ |
| | | | | | | | | | p < 0.05 |
| 5379 | Kidney | 1 | 1 | TRUE | 0.088546 | 0.097583 | 4.28 | 1.878193 | NA |
| 5381 | Kidney | 0.777778 | 1 | TRUE | 0.209592 | 0.21451 | 1.98 | 0.590401 | NA |
| 5390 | Kidney | 0.927083 | 0.782946 | TRUE | 0.204979 | 0.212844 | 2.06 | 1.58518 | NA |
| 5394 | Kidney | 0.885417 | 0.224806 | TRUE | 0.268885 | 0.274031 | 1.51 | 0.685739 | NA |
| 5399 | Kidney | 0.756944 | 1 | TRUE | 0.130723 | 0.140753 | 3.17 | 0.498509 | NA |
| 5400 | Kidney | 0.788194 | 0.992248 | TRUE | 0.14807 | 0.157818 | 2.91 | 1.677708 | NA |
| 5401 | Kidney | 0.868056 | 0.085271 | TRUE | 0.116875 | 0.123745 | 3.52 | 0.567266 | NA |
| 5409 | Kidney | 0.84375 | 0.031008 | TRUE | 0.088424 | 0.100013 | 4.2 | 0.429357 | NA |
| 5414 | Kidney | 0.90625 | 0.620155 | TRUE | 0.128241 | 0.135332 | 3.28 | 1.821217 | NA |
| 5415 | Kidney | 0.854167 | 0.589147 | TRUE | 0.13343 | 0.140753 | 3.16 | 1.779232 | NA |
| 5447 | Kidney | 1 | 1 | TRUE | 0.1521 | 0.159597 | 2.76 | 0.536905 | NA |
| 5454 | Kidney | 1 | 1 | TRUE | 0.078685 | 0.091987 | 4.5 | 0.465013 | NA |
| 5456 | Kidney | 0.725694 | 1 | TRUE | 0.282511 | 0.287794 | 1.37 | 0.664079 | NA |
| 5467 | Kidney | 0.927083 | 1 | TRUE | 0.23248 | 0.237226 | 1.79 | 1.501941 | NA |
| 5473 | Kidney | 1 | 1 | TRUE | 0.153405 | 0.159597 | 2.73 | 1.730286 | NA |
| 5478 | Kidney | 0.947917 | 0.860465 | TRUE | 0.15479 | 0.162831 | 2.67 | 1.767865 | NA |
| 5482 | Kidney | 0.913194 | 0.844961 | TRUE | 0.001611 | 0.003144 | 13.19 | 3.036536 | 0.001 |
| | | | | | | | | | a ‰¤ |
| | | | | | | | | | p < 0.01 |
| 5484 | Kidney | 0.979167 | 0.821705 | TRUE | 0.369794 | 0.372976 | 0.85 | 1.322226 | NA |
| 5490 | Kidney | 0.923611 | 0.821705 | TRUE | 0.354309 | 0.358236 | 0.93 | 0.740876 | NA |
| 5493 | Kidney | 0.829861 | 0.511628 | TRUE | 0.1552 | 0.162831 | 2.65 | 1.656177 | NA |
| 5501 | Kidney | 0.708333 | 0.217054 | TRUE | 0.1552 | 0.164291 | 2.62 | 1.637356 | NA |
| 5503 | Kidney | 0.795139 | 0.395349 | TRUE | 0.142308 | 0.150077 | 3.02 | 1.698825 | NA |
| 5507 | Kidney | 0.777778 | 0.736434 | TRUE | 0.075815 | 0.084931 | 4.71 | 1.927322 | NA |
| 5511 | Kidney | 0.993056 | 1 | TRUE | 0.195692 | 0.201757 | 2.16 | 0.640442 | NA |
| 5532 | Kidney | 0.958333 | 0.937981 | TRUE | 0.0264 | 0.034103 | 7.32 | 2.264633 | 0.01 |
| | | | | | | | | | a ‰¤ |
| | | | | | | | | | p < 0.05 |
| 6294 | Liver | 1 | 1 | TRUE | 0.13491 | 0.140753 | 3.18 | 0.723199 | NA |
| 6304 | Liver | 0.902439 | 1 | TRUE | 0.210398 | 0.21429 | 2 | 1.286327 | NA |
| 6313 | Liver | 1 | 1 | TRUE | 8.87E-04 | 0.001249 | 15.43 | 0.490882 | 0.001 |
| | | | | | | | | | a ‰¤ |
| | | | | | | | | | p < 0.01 |
| 6314 | Liver | 0.766938 | 0.24 | TRUE | 0.369794 | 0.372976 | 0.87 | 0.848081 | NA |
| 6322 | Liver | 0.891599 | 0.36 | TRUE | 0.119797 | 0.123745 | 3.53 | 0.706634 | NA |
| 6331 | Liver | 0.826558 | 0.62 | TRUE | 0.236096 | 0.240396 | 1.75 | 0.783901 | NA |
| 6334 | Liver | 0.861789 | 0.46 | TRUE | 0.272098 | 0.274958 | 1.5 | 0.804417 | NA |
| 6337 | Liver | 1 | 1 | TRUE | 0.276623 | 0.280905 | 1.44 | 0.808192 | NA |
| 6312 | Liver | 0.872629 | 0.34 | TRUE | 0.010431 | 0.011938 | 9.72 | 0.563775 | 0.01 |
| | | | | | | | | | a ‰¤ |
| | | | | | | | | | p < 0.05 |
| 6345 | Liver | 0.872629 | 0.7 | TRUE | 0.208959 | 0.213355 | 2.03 | 0.772614 | NA |
| 7175 | Lung | 0.986355 | 0.834862 | TRUE | 0.044765 | 0.050231 | 6.1 | 0.686475 | NA |
| 7176 | Lung | 0.717349 | 0 | TRUE | 0.052186 | 0.057186 | 5.68 | 0.671873 | NA |
| 7178 | Lung | 0.894737 | 0.495413 | TRUE | 0.232761 | 0.237226 | 1.78 | 1.231556 | NA |
| 7183 | Lung | 0.71345 | 0.055046 | TRUE | 0.003366 | 0.004174 | 12.18 | 0.531228 | 0.001 |
| | | | | | | | | | a ‰¤ |
| | | | | | | | | | p < 0.01 |
| 7203 | Lung | 0.992203 | 1 | TRUE | 0.018576 | 0.021025 | 8.29 | 1.538316 | 0.01 |
| | | | | | | | | | a ‰¤ |
| | | | | | | | | | p < 0.05 |
| 7212 | Lung | 0.810916 | 0.302752 | TRUE | 0.052173 | 0.057186 | 5.7 | 0.656175 | NA |
| 7213 | Lung | 1 | 1 | TRUE | 0.015447 | 0.0177 | 8.67 | 1.54809 | 0.01 |
| | | | | | | | | | a ‰¤ |
| | | | | | | | | | p < 0.05 |
| 7220 | Lung | 0.707602 | 0.110092 | TRUE | 0.221204 | 0.224134 | 1.91 | 1.234682 | NA |
| 7226 | Lung | 0.768031 | 1 | TRUE | 0.015447 | 0.0177 | 8.66 | 1.572267 | 0.01 |
| | | | | | | | | | a ‰¤ |
| | | | | | | | | | p < 0.05 |
| 7236 | Lung | 1 | 1 | TRUE | 0.04226 | 0.045536 | 6.42 | 1.527423 | 0.01 |
| | | | | | | | | | a ‰¤ |
| | | | | | | | | | p < 0.05 |
| 7239 | Lung | 0.877193 | 0.183486 | TRUE | 0.046208 | 0.051488 | 5.99 | 0.687264 | NA |
| 7249 | Lung | 0.988304 | 0.990826 | TRUE | 0.208959 | 0.213355 | 2.03 | 0.809328 | NA |
| 7258 | Lung | 0.824561 | 0.229358 | TRUE | 0.130723 | 0.135332 | 3.27 | 0.761691 | NA |
| 7269 | Lung | 0.840156 | 1 | TRUE | 0.003118 | 0.003856 | 12.57 | 1.759556 | 0.001 |
| | | | | | | | | | a ‰¤ |
| | | | | | | | | | p < 0.01 |
| 7274 | Lung | 0.988304 | 0.889908 | TRUE | 0.003118 | 0.003856 | 12.5 | 0.527243 | 0.001 |
| | | | | | | | | | a ‰¤ |
| | | | | | | | | | p < 0.01 |
| 7275 | Lung | 0.719298 | 0.577982 | TRUE | 0.014736 | 0.016976 | 8.84 | 1.572059 | 0.01 |
| | | | | | | | | | a ‰¤ |
| | | | | | | | | | p < 0.05 |
| 7276 | Lung | 0.760234 | 0.311927 | TRUE | 0.3193 | 0.321885 | 1.12 | 0.85269 | NA |
| 7298 | Lung | 0.982456 | 1 | TRUE | 0.302782 | 0.306228 | 1.23 | 0.844478 | NA |
| 7300 | Lung | 0.822612 | 0.495413 | TRUE | 0.063003 | 0.068108 | 5.29 | 1.406562 | NA |
| 7316 | Lung | 0.966862 | 1 | TRUE | 0.057214 | 0.061057 | 5.51 | 1.428661 | NA |
| 7318 | Lung | 0.992203 | 1 | TRUE | 0.09096 | 0.097583 | 4.3 | 0.726649 | NA |
| 7321 | Lung | 0.826511 | 0.862385 | TRUE | 0.008645 | 0.009671 | 10.4 | 0.61747 | 0.001 |
| | | | | | | | | | a ‰¤ |
| | | | | | | | | | p < 0.01 |
| 8069 | Lung | 0.861446 | 0 | TRUE | 0.128241 | 0.131292 | 3.37 | 1.288964 | NA |
| 8070 | Lung | 0.795181 | 0 | TRUE | 0.10303 | 0.108268 | 3.92 | 1.322163 | NA |
| 8073 | Lung | 0.953815 | 0.055046 | TRUE | 0.190633 | 0.193434 | 2.25 | 0.809216 | NA |
| 8075 | Lung | 0.761044 | 0.009174 | TRUE | 0.013681 | 0.015659 | 9.04 | 1.534013 | 0.01 |
| | | | | | | | | | a ‰¤ |
| | | | | | | | | | p < 0.05 |
| 8076 | Lung | 0.993976 | 0.834862 | TRUE | 0.274121 | 0.27864 | 1.46 | 1.202998 | NA |
| 8077 | Lung | 1 | 0.889908 | TRUE | 0.122135 | 0.124845 | 3.49 | 1.29515 | NA |
| 8082 | Lung | 0.74498 | 0.009174 | TRUE | 0.103742 | 0.108268 | 3.84 | 1.322933 | NA |
| 8083 | Lung | 0.87751 | 0.045872 | TRUE | 0.04356 | 0.046062 | 6.33 | 1.433764 | 0.01 |
| | | | | | | | | | a ‰¤ |
| | | | | | | | | | p < 0.05 |
| 8087 | Lung | 0.98996 | 1 | TRUE | 0.011726 | 0.012683 | 9.55 | 0.652157 | 0.01 |
| | | | | | | | | | a ‰¤ |
| | | | | | | | | | p < 0.05 |
| 8090 | Lung | 0.995984 | 0.990826 | TRUE | 0.002601 | 0.003144 | 13.27 | 1.666139 | 0.001 |
| | | | | | | | | | a ‰¤ |
| | | | | | | | | | p < 0.01 |
| 8092 | Lung | 0.997992 | 0.990826 | TRUE | 0.310729 | 0.313215 | 1.18 | 0.859757 | NA |
| 8093 | Lung | 0.959839 | 0.183486 | TRUE | 0.122599 | 0.125538 | 3.46 | 1.318887 | NA |
| 8110 | Lung | 1 | 1 | TRUE | 0.077563 | 0.084931 | 4.72 | 0.710709 | NA |
| 8111 | Lung | 0.945783 | 0.302752 | TRUE | 0.033509 | 0.037638 | 7.05 | 1.44971 | 0.01 |
| | | | | | | | | | a ‰¤ |
| | | | | | | | | | p < 0.05 |
| 8116 | Lung | 0.817269 | 0.12844 | TRUE | 0.10859 | 0.112921 | 3.75 | 1.343025 | NA |
| 8117 | Lung | 0.875502 | 0.293578 | TRUE | 0.182732 | 0.185078 | 2.35 | 1.236864 | NA |
| 8124 | Lung | 0.933735 | 0.311927 | TRUE | 0.040778 | 0.04352 | 6.59 | 1.429285 | 0.01 |
| | | | | | | | | | a ‰¤ |
| | | | | | | | | | p < 0.05 |
| 8130 | Lung | 0.783133 | 0.174312 | TRUE | 0.232761 | 0.237226 | 1.79 | 1.219027 | NA |
| 8140 | Lung | 0.931727 | 0.605505 | TRUE | 0.153405 | 0.159597 | 2.78 | 1.291264 | NA |
| 8151 | Lung | 0.997992 | 1 | TRUE | 0.09692 | 0.103313 | 4.05 | 1.321182 | NA |
| 8176 | Lung | 1 | 1 | TRUE | 0.010431 | 0.011427 | 9.87 | 0.64453 | 0.01 |
| | | | | | | | | | a ‰¤ |
| | | | | | | | | | p < 0.05 |
| 8178 | Lung | 0.96988 | 0.788991 | TRUE | 0.307849 | 0.310171 | 1.2 | 1.16786 | NA |
| 8184 | Lung | 0.917671 | 1 | TRUE | 0.096837 | 0.103313 | 4.07 | 0.74462 | NA |
| 8211 | Lung | 1 | 1 | TRUE | 0.14757 | 0.152051 | 2.99 | 0.768758 | NA |
| 8218 | Lung | 0.759036 | 0.100917 | TRUE | 0.103351 | 0.108268 | 3.87 | 1.335409 | NA |
| 8222 | Lung | 1 | 0.990826 | TRUE | 0.171705 | 0.178449 | 2.46 | 1.251303 | NA |
| 8227 | Lung | 0.953815 | 0.779817 | TRUE | 0.302782 | 0.306228 | 1.24 | 1.167536 | NA |
| 8229 | Lung | 0.98996 | 0.807339 | TRUE | 0.090121 | 0.097583 | 4.34 | 1.35274 | NA |
| 8238 | Lung | 0.977912 | 0.825688 | TRUE | 0.1521 | 0.159597 | 2.84 | 0.788829 | NA |
| 8242 | Lung | 1 | 1 | TRUE | 0.210398 | 0.21429 | 1.99 | 1.224765 | NA |
| 8251 | Lung | 0.712851 | 0.577982 | TRUE | 0.283599 | 0.287794 | 1.37 | 1.202498 | NA |
| 8257 | Lung | 1 | 1 | TRUE | 0.190633 | 0.193434 | 2.25 | 1.230688 | NA |
| 8270 | Lung | 0.787149 | 0.229358 | TRUE | 0.094414 | 0.100013 | 4.22 | 1.347786 | NA |
| 8977 | Prostate | 0.995951 | 0.941176 | TRUE | 0.077563 | 0.108268 | 3.92 | 0.208337 | NA |
| 8984 | Prostate | 0.809717 | 0.352941 | TRUE | 0.1552 | 0.185078 | 2.37 | 3.392262 | NA |
| 8992 | Prostate | 0.969636 | 0.529412 | TRUE | 0.160027 | 0.185078 | 2.38 | 0.343438 | NA |
| 9862 | Stomach | 0.90799 | 0.416667 | TRUE | 0.451477 | 0.45418 | 0.59 | 1.132645 | NA |
| 9892 | Stomach | 0.98063 | 1 | TRUE | 0.096837 | 0.103313 | 4.09 | 0.710699 | NA |
| 10760 | Thyroid | 0.849206 | 0.033898 | TRUE | 0.096837 | 0.108268 | 3.84 | 2.67015 | NA |
| | Gland | | | | | | | | |
| 10767 | Thyroid | 0.876984 | 0.101695 | TRUE | 0.077346 | 0.09717 | 4.38 | 2.954 | NA |
| | Gland | | | | | | | | |
| 10783 | Thyroid | 0.944444 | 0.440678 | TRUE | 0.32023 | 0.328182 | 1.09 | 0.590782 | NA |
| | Gland | | | | | | | | |
| 11650 | Endometrium | 0.966667 | 1 | TRUE | 0.253804 | 0.261968 | 1.6 | 0.634688 | NA |
| 11652 | Endometrium | 0.883333 | 1 | TRUE | 0.063003 | 0.076293 | 5.06 | 0.440063 | NA |
| 11662 | Endometrium | 0.838889 | 1 | TRUE | 0.42002 | 0.423951 | 0.68 | 0.74928 | NA |
| 11664 | Endometrium | 0.761111 | 1 | TRUE | 0.328998 | 0.337322 | 1.04 | 1.428949 | NA |
| 11674 | Endometrium | 1 | 0.869565 | TRUE | 0.046208 | 0.057186 | 5.69 | 0.411612 | NA |
| 11679 | Endometrium | 0.788889 | 0.043478 | TRUE | 0.150279 | 0.159597 | 2.85 | 1.836213 | NA |
| 11685 | Endometrium | 0.877778 | 0.130435 | TRUE | 0.204163 | 0.212844 | 2.07 | 1.669337 | NA |
| 11714 | Endometrium | 0.772222 | 0.086957 | TRUE | 0.1521 | 0.159597 | 2.75 | 1.83321 | NA |

In some embodiments, the nORF is not HOXB-AS3.

In some embodiments, the cancer is not colorectal cancer.

In some embodiments, the nORF is not PINT87aa (LINC-PINT).

In some embodiments, the cancer is not glioblastoma.

### EXAMPLES

The following examples further illustrate the invention but should not be construed as in any way limiting its scope.

### Example 1

nORFs are Pervasively Translated and Important for Further Investigation

nORFs are typically smaller than canonical ORFs, the peptides or micro-proteins they encode are particularly attractive as putative allosteric cellular regulators, due to their size and the potential specificity of peptide interactions. Therefore, because the accepted nomenclature itself is inconsistent, we classified and catalogued all human nORFs from various sources, prioritizing those with strong evidence for translation and distinguishing between nORFs that are in frame and out of frame with overlapping canonical ORFs and released it as an open-source database (norfs.org/home).

Identifying and Characterizing Transcripts Encoding nORFs

To identify transcripts encoding nORFs (nORF transcripts), we extracted genomic coordinates of transcripts quantified in the UCSC Toil pipeline from the GENCODE v23 reference genome annotation and compared these with the genomic coordinates of nORFs acquired from the curated nORFs.org database, using a custom pipeline (FIG. 5). All nORFs present in the database had strong experimental evidence for translation from mass spectrometry or ribosome sequencing. We used GffCompare to identify transcripts and nORFs with compatible intron chains and compared genomic coordinates to retain only transcript-nORF mappings where a nORF is completely contained within the transcript genomic start and end position. We considered only nORFs encoded by noncoding transcripts. This resulted in the identification of 1,488 nORF transcripts.

To determine if nORF transcripts are expressed in any tissue included in the study, we defined an expression threshold of 0.5 counts per million (CPM) across at least 10% of a single tissue. This allowed us to prioritize transcripts that are more likely to be accurately quantified and expressed at a biologically meaningful level. Using this threshold, we identified 926 expressed nORF transcripts for inclusion in this study.

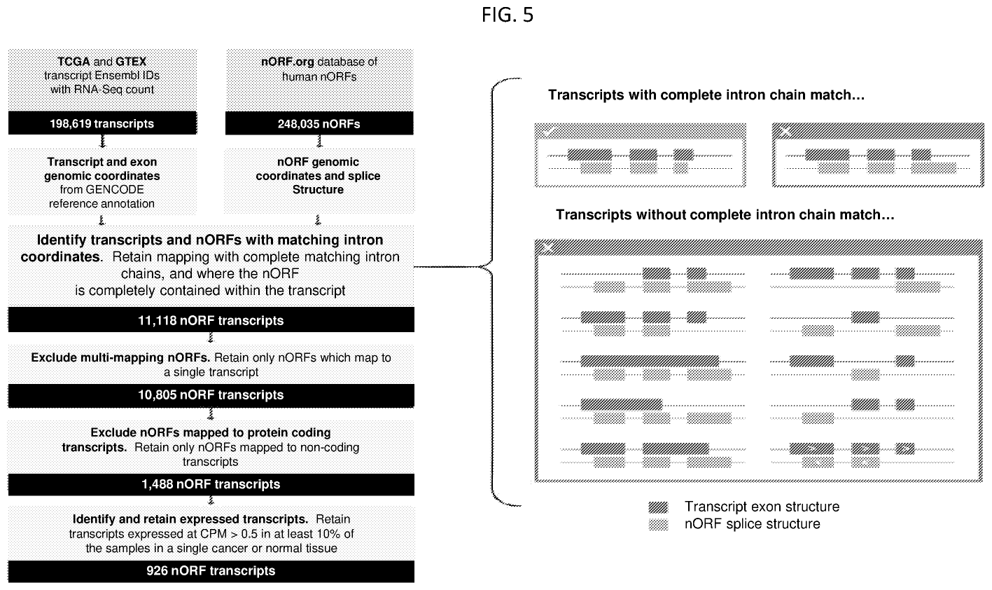

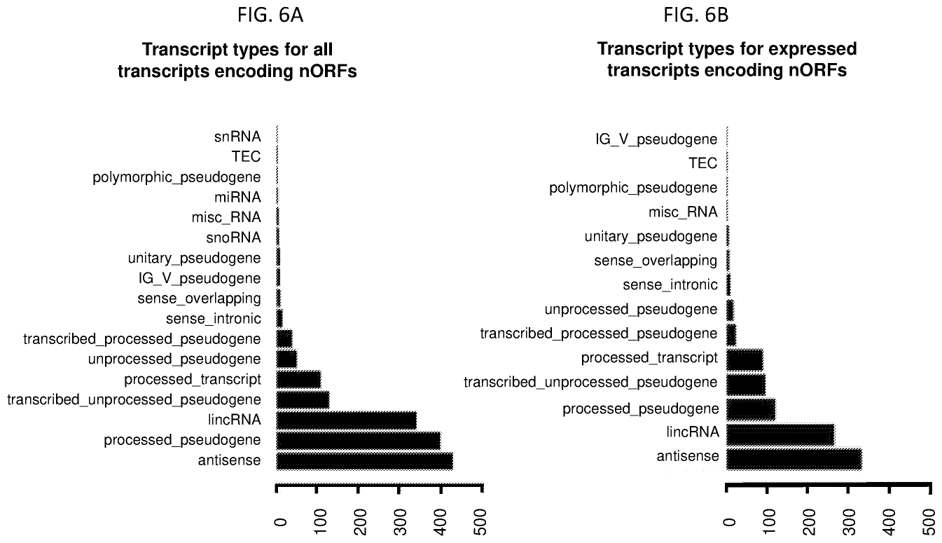

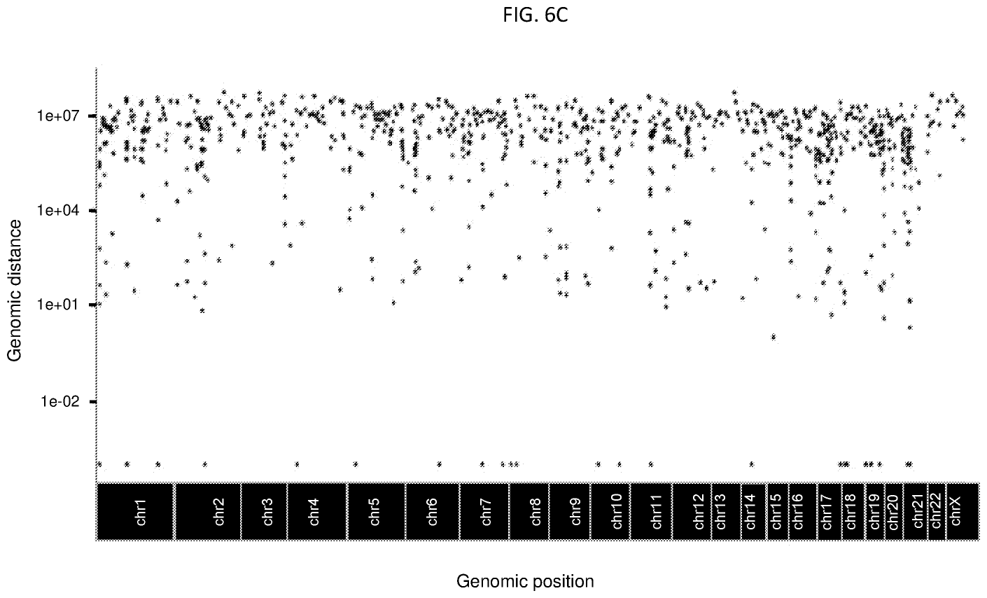

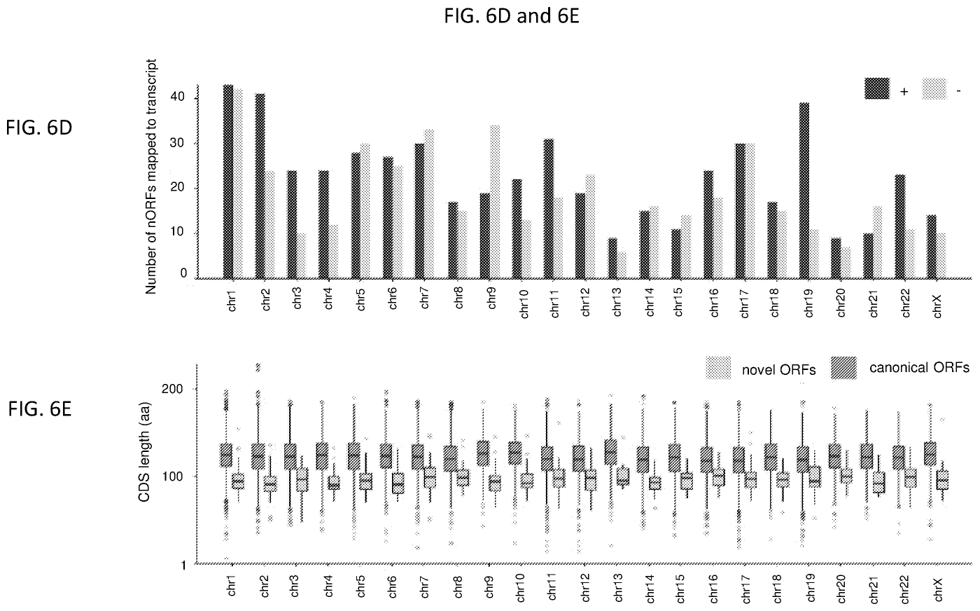

We characterized the genomic properties of all nORF transcripts (FIG. 6A) and the 926 nORF transcripts (FIG. 6B) included in this study, by genomic coordinates and biotype annotation.

We considered genomic distribution and strand bias (FIGS. 6C and 6D) to ensure there was no substantial bias in genomic location for the nORF transcripts considered in this study. Across autosomal chromosomes nORF transcripts were consistently distributed, with a small number of nORFs sharing the same start site. However, no transcripts encoding nORFs were identified on the Y chromosome—this is consistent with the lower abundance of genes present on this chromosome. Whilst some chromosomes did exhibit strong strand bias in the number of nORF transcripts identified, namely chromosome 19, overall transcripts were identified consistently in both genomic strands. Comparing the length of novel and canonical ORFs (FIG. 6E) revealed a degree of overlap in length, but median nORF length was substantially below that of canonical ORFs, with the majority of nORFs encoding proteins less than 100 amino acids in length.

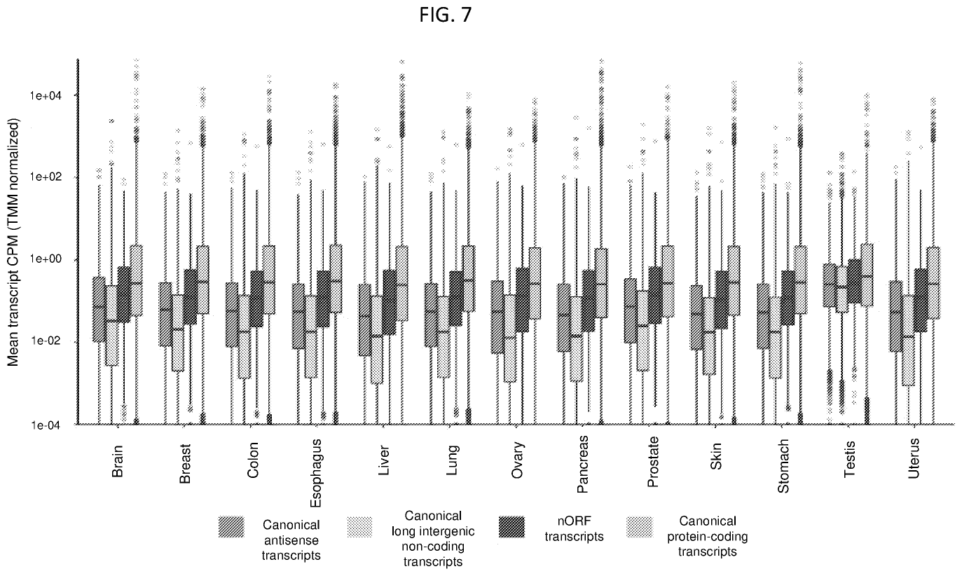

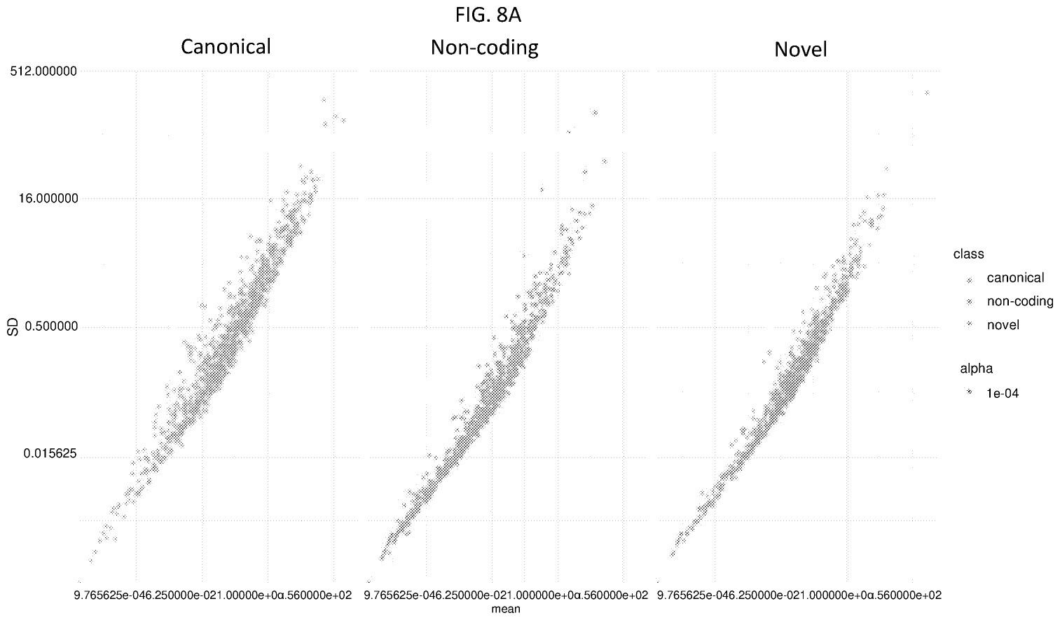

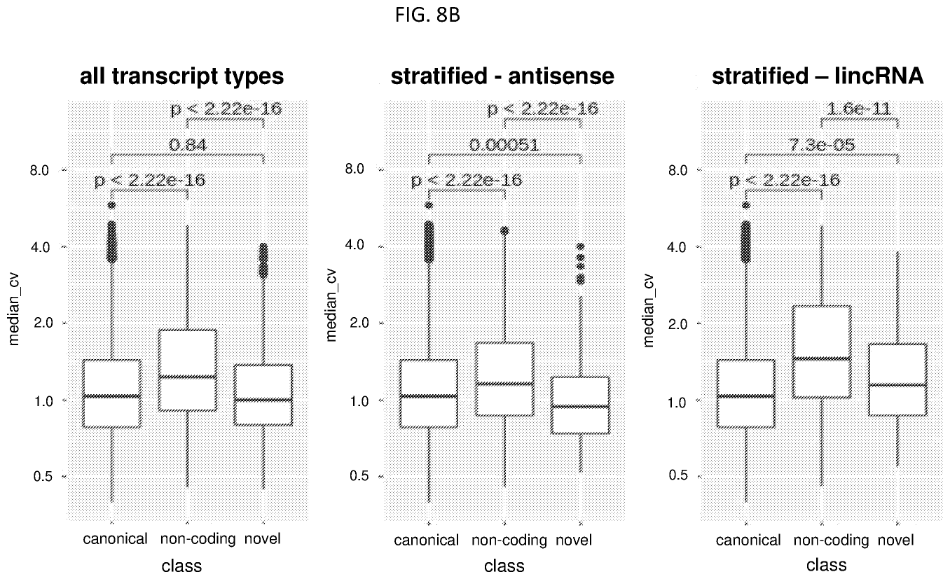

Following identification of nORF transcripts, we evaluated transcript mean expression across all GTEx normal tissues included in this study. We showed mean nORF transcript expression compared with canonical protein-coding transcripts and also compared against canonical antisense and lincRNA expression—as these are the two main transcript classifications within which nORF transcripts are identified (FIGS. 7, 8A, and 8B). The median expression of nORF transcripts was below that of canonical protein-coding transcripts, but above that of both noncoding RNA classes. We considered that many nORF transcripts have mean expression comparable with that observed in protein-coding transcripts, which provides confidence that transcripts encoding nORFs may be expressed at an adequate level for translation to occur.

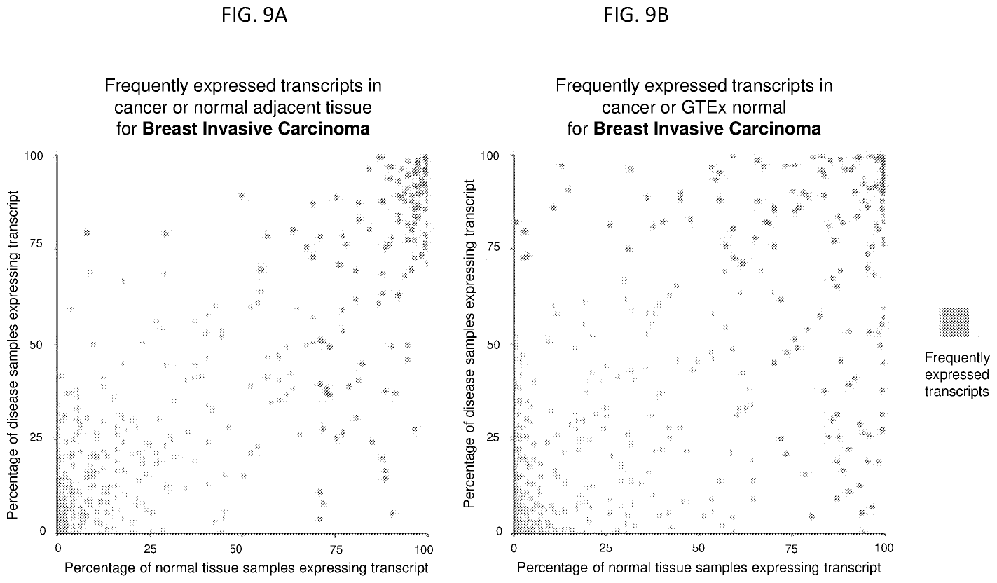

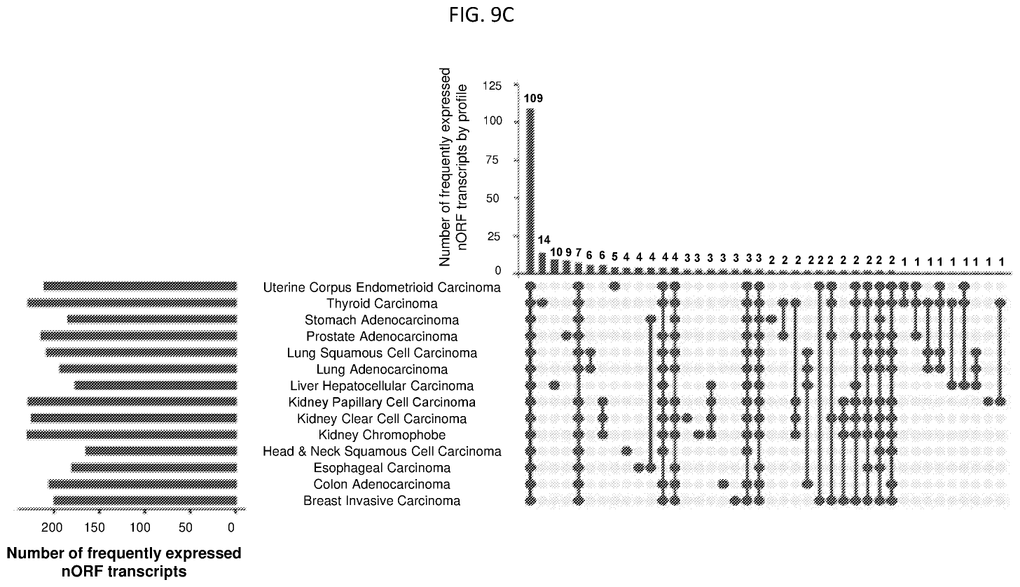

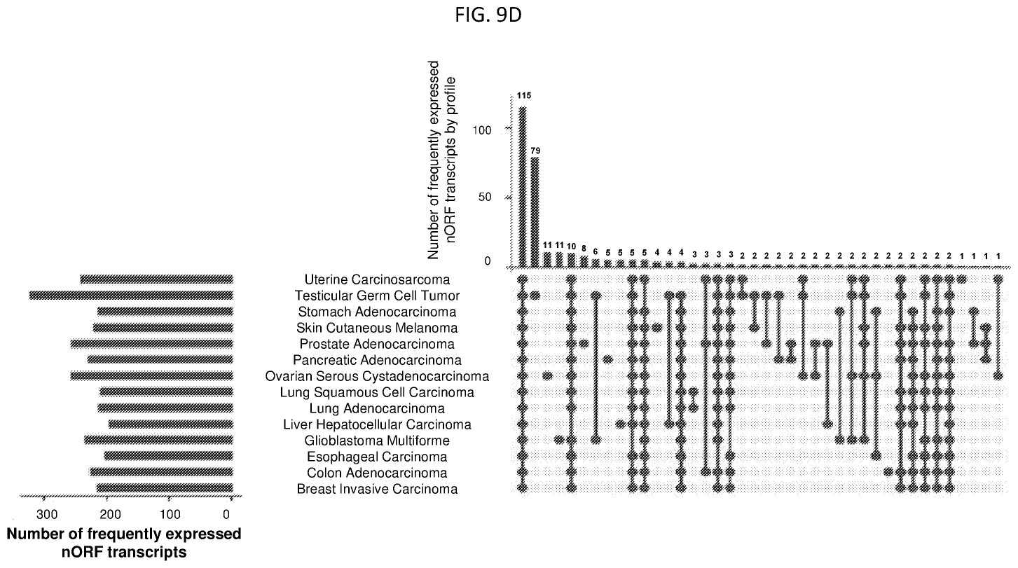

Many nORF transcripts were poorly expressed, with mean CPM values below 0.5. We identified and prioritized nORF transcripts frequently expressed in cancer tissues or the corresponding NAT or GTEx normal tissue. Both cancer and reference normal tissues were considered when identifying frequently expressed nORF transcripts, as we aimed to capture nORF transcripts both up- and down-regulated between cancer and normal tissues. Frequently expressed nORF transcripts were defined as having CPM greater than 0.5 across at least 70% of samples in either cancer or corresponding reference tissue. A representative distribution of expression across samples in cancer tissue and corresponding NAT (FIG. 9A) and GTEx normal tissue (FIG. 9B) is shown to illustrate this threshold for frequent expression. Two observations provided confidence that a suitable expression threshold had been selected: (i) expression was largely binary, with most nORF transcripts expressed in either every sample or no samples in a tissue (ii) the number of samples in cancer and normal tissue expressing a given nORF transcript were highly correlated.

When comparing cancer with NAT, we determined 359 out of 926 nORF transcripts were frequently expressed in at least one cancer type; when comparing with GTEx normal tissue, 464 out of 926 nORF transcripts were frequently expressed in at least one cancer type. The number of frequently expressed nORF transcripts identified was consistent across cancer types (FIGS. 9C and 9D).

A large proportion of nORF transcripts were frequently expressed across all cancer types—109 nORF transcripts for cancer and NAT; 115 nORF transcripts for cancer and GTEx normal tissue. On the other hand, comparatively few nORF transcripts were frequently expressed in any particular subset of cancer types—for example, just 14 nORF transcripts were only frequently expressed in thyroid carcinoma or thyroid NAT. This likely reflects consistent expression of nORF transcripts across tissues. A disproportionate number of nORF transcripts (79) are frequently expressed only in testicular germ cell tumor tissue or GTEx testis tissue, which is consistent with mean transcript expression patterns in testis tissue (FIGS. 8A and 8B)—noncoding transcript expression in the testis appears unusually distinct compared with other tissues.

Identifying Differentially Expressed nORF Transcripts

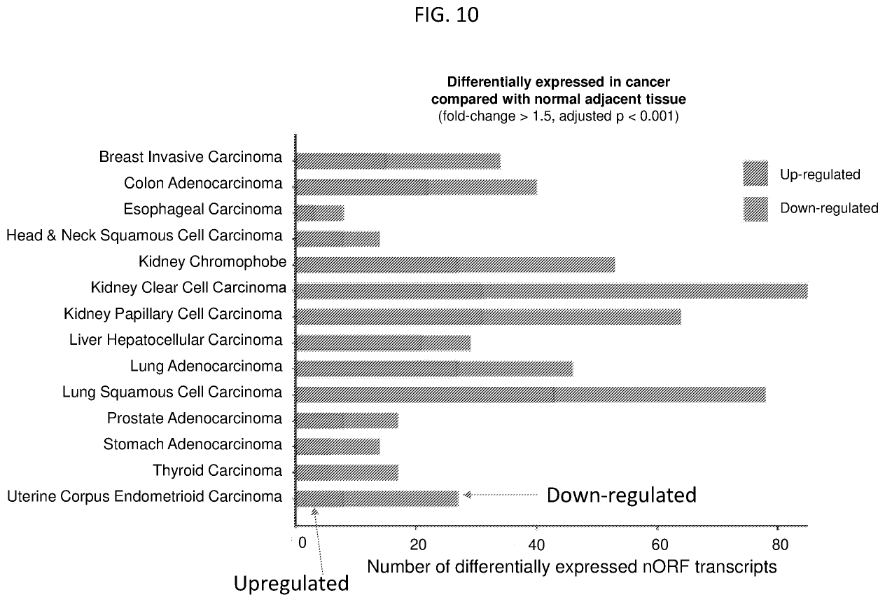

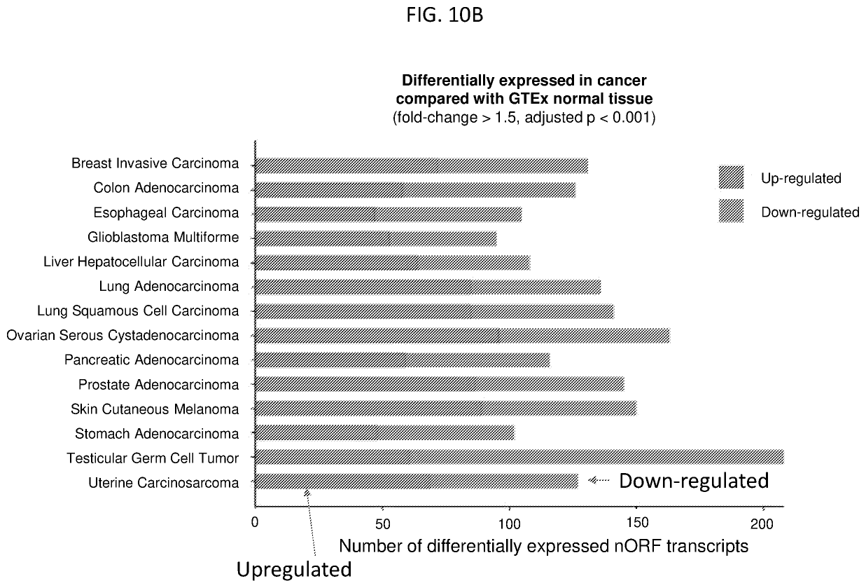

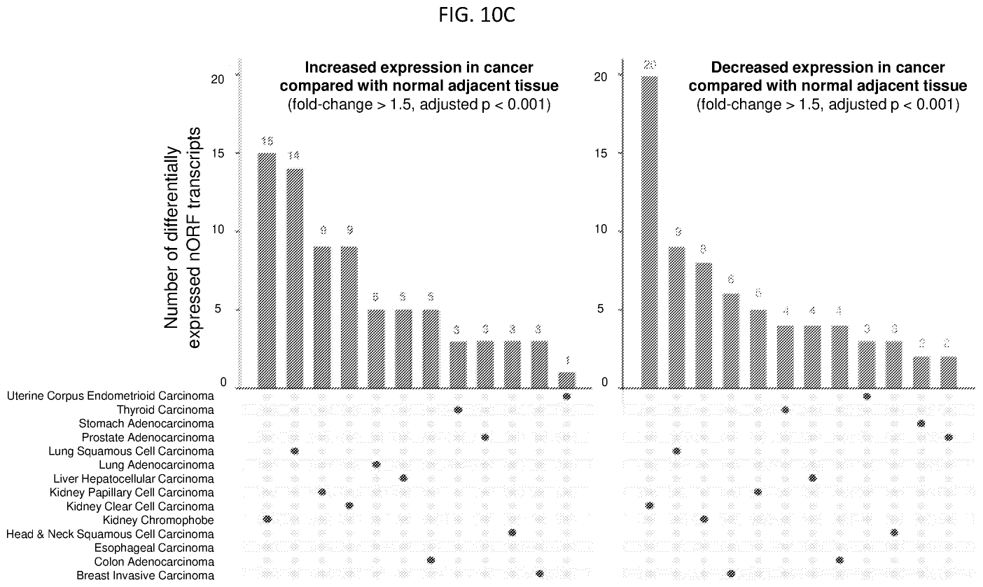

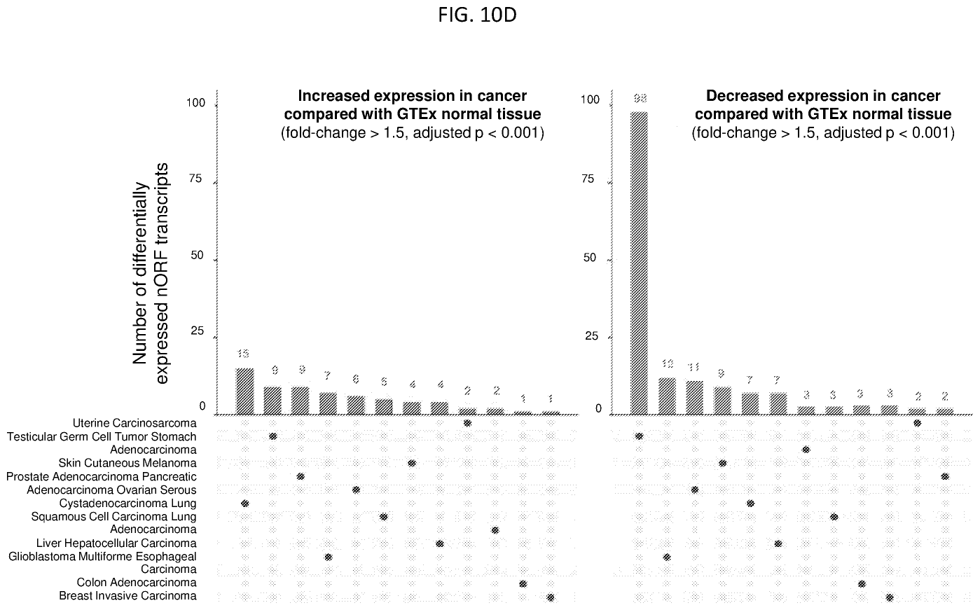

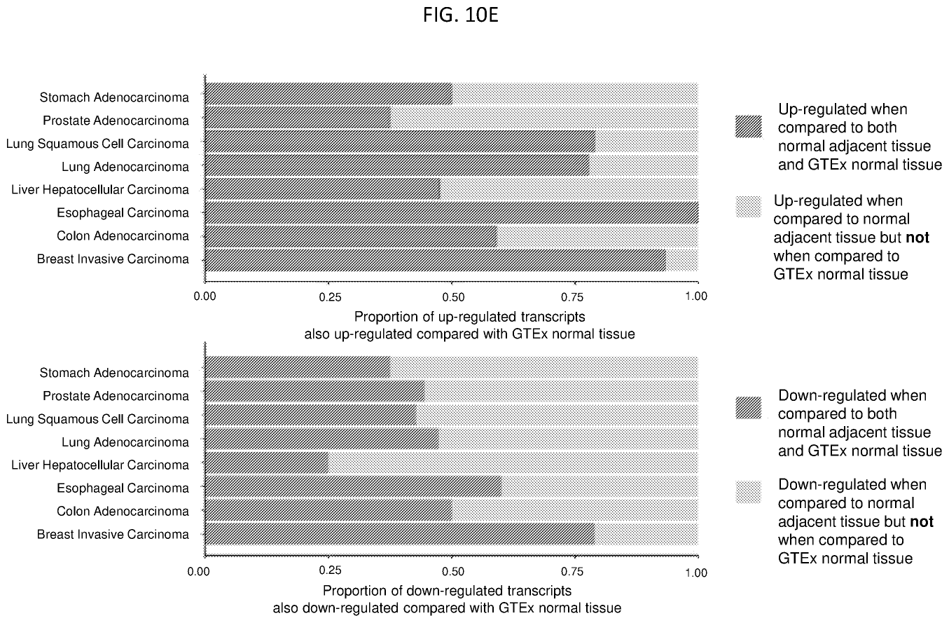

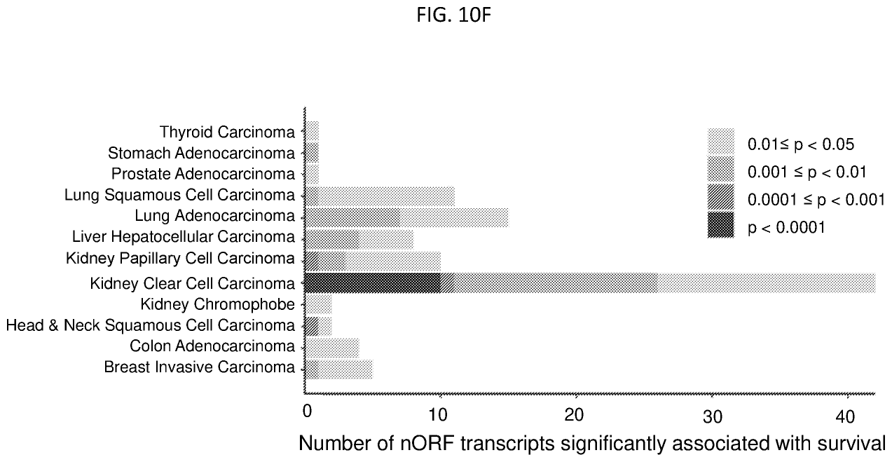

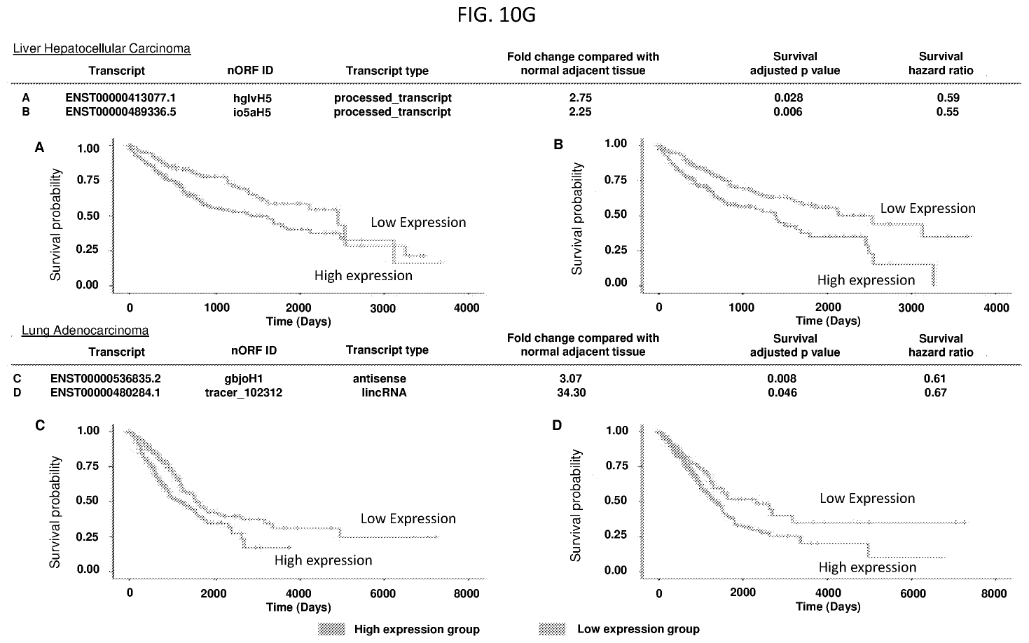

To identify nORF transcripts dysregulated in cancer, we performed differential expression analysis for cancer compared with either NAT or GTEx normal tissue. We normalized RNA-Seq expected counts from the UCSC Toil dataset using the trimmed mean of M-values (TMM) method and performed differential expression analysis using the general linear model (GLM) framework provided by edgeR, as described in Materials and Methods. A fold change threshold of 2 and adjusted p value threshold of 0.001 were used to call differentially expressed nORF transcripts. Only frequently expressed nORF transcripts were considered. Corresponding analysis using a fold change threshold of 1.5 is provided in FIG. 10.

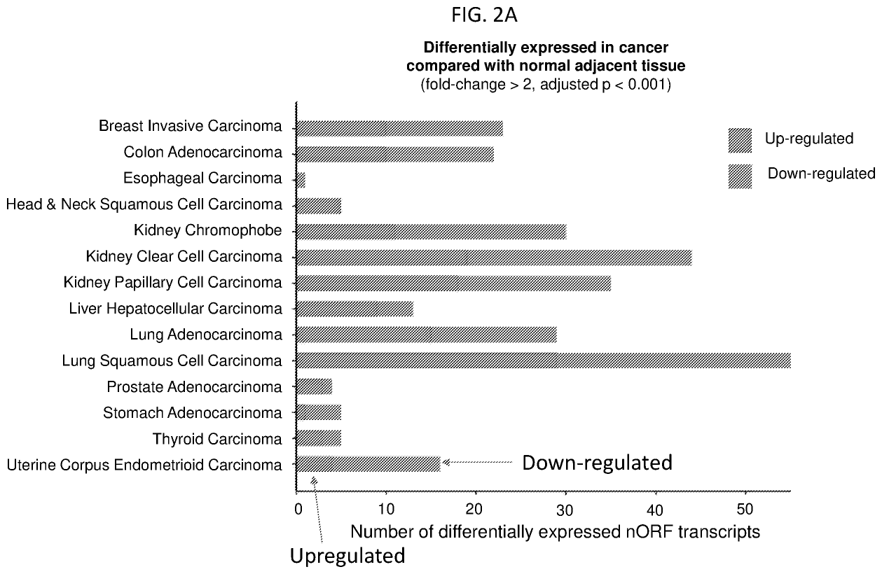

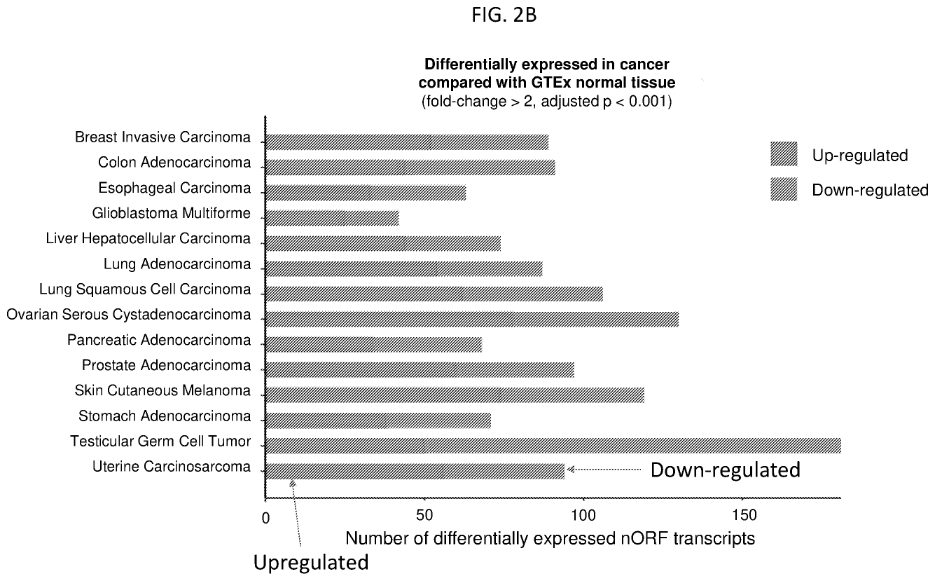

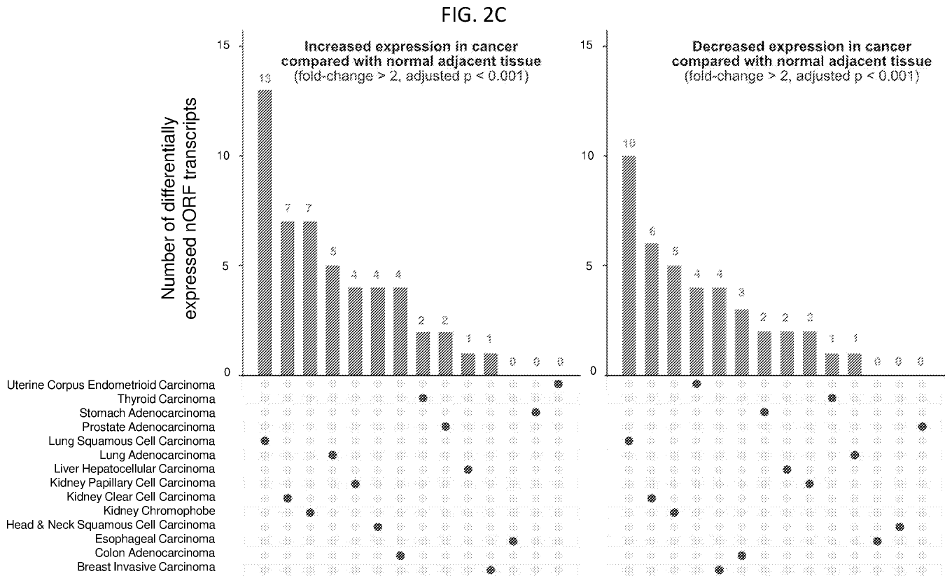

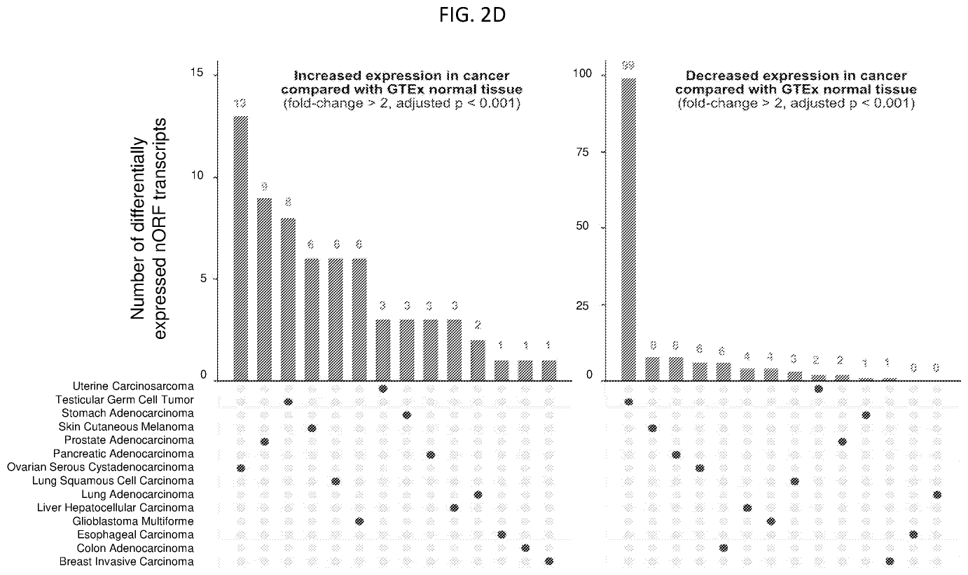

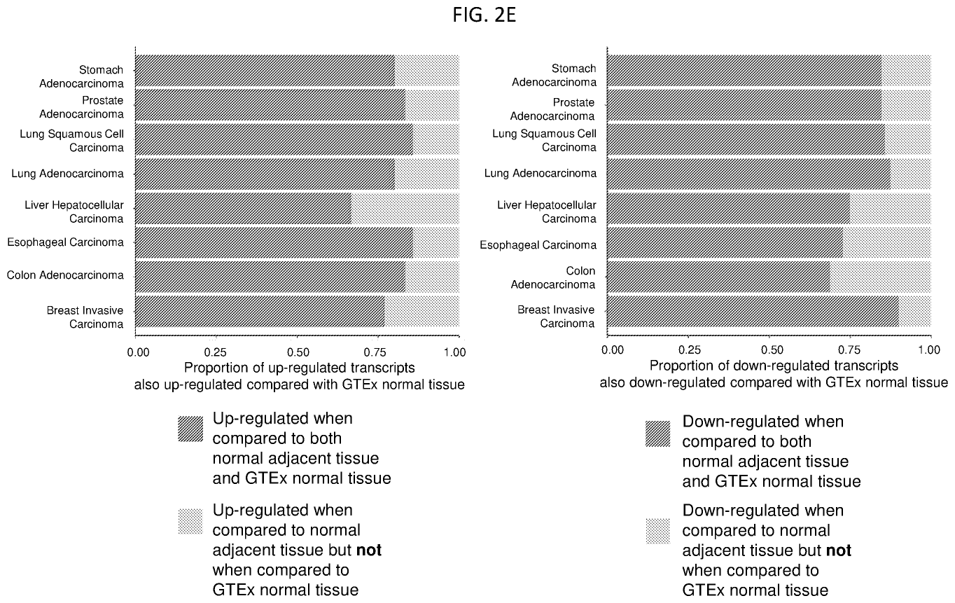

This analysis revealed 152 nORF transcripts as dysregulated in at least a single cancer type when comparing cancer with NAT (FIG. 2A), and 386 were dysregulated when compared with GTEx normal tissue (FIG. 2B). This represented a large proportion of the total number of frequently expressed nORF transcripts. Whilst the number of frequently expressed nORF transcripts was consistent across cancer types, the number of nORF transcripts differentially expressed in each cancer type was diverse. Some cancer types exhibited far more extensive dysregulation of nORF transcription, namely kidney clear cell carcinoma and lung squamous cell carcinoma.

We observed a limited number of nORF transcripts with cancer-type specific dysregulation. In lung squamous cell carcinoma 13 nORF transcripts were uniquely upregulated, and 10 uniquely down-regulated, when compared against NAT. Kidney clear cell carcinoma, kidney chromophobe and testicular germ cell tumors also exhibited a large degree of cancer-type specific dysregulation (FIGS. 2C and 2D). Overall, these results demonstrated widespread dysregulation of nORF transcripts across cancers.

To assess the reproducibility of differential expression results when comparing against NAT or GTEx normal tissue, we investigated differentially expressed nORF transcripts identified in eight cancer types with both types of reference normal tissue. Differential expression relative to GTEx normal tissue consistently revealed a larger number of dysregulated nORF transcripts. Most cancer types showed highly reproducible differential expression results between the two reference normal tissues (FIG. 2E). Controlling for confounding factors such as age, sex and ethnicity may help improve the reliability and reproducibility of this differential expression analysis. A degree of discrepancy was expected, as (i) NAT is affected by the tumor microenvironment (ii) GTEx normal tissues are more highly represented with larger sample sizes. However, in all but one disease at least 75% of nORF transcripts identified as differentially expressed when using NAT as a reference tissue are also identified when using GTEx normal tissue.

### Prognostic Value of Differentially Expressed Transcripts

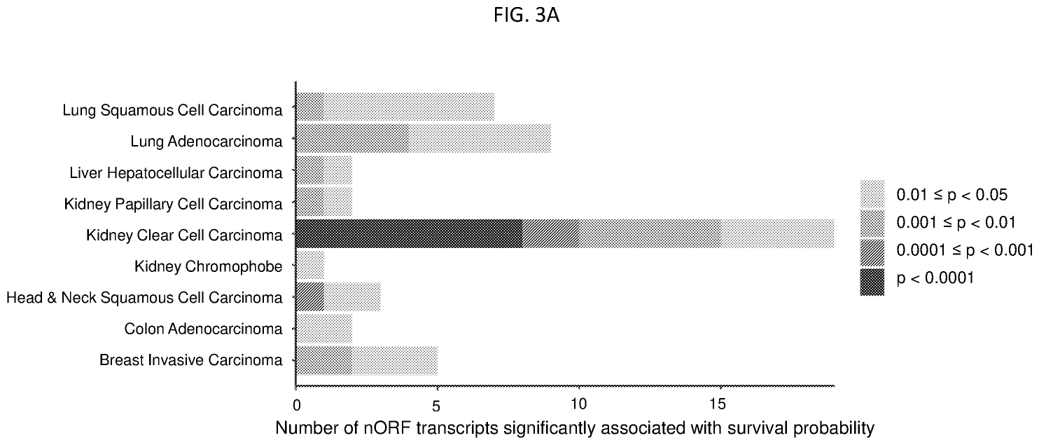

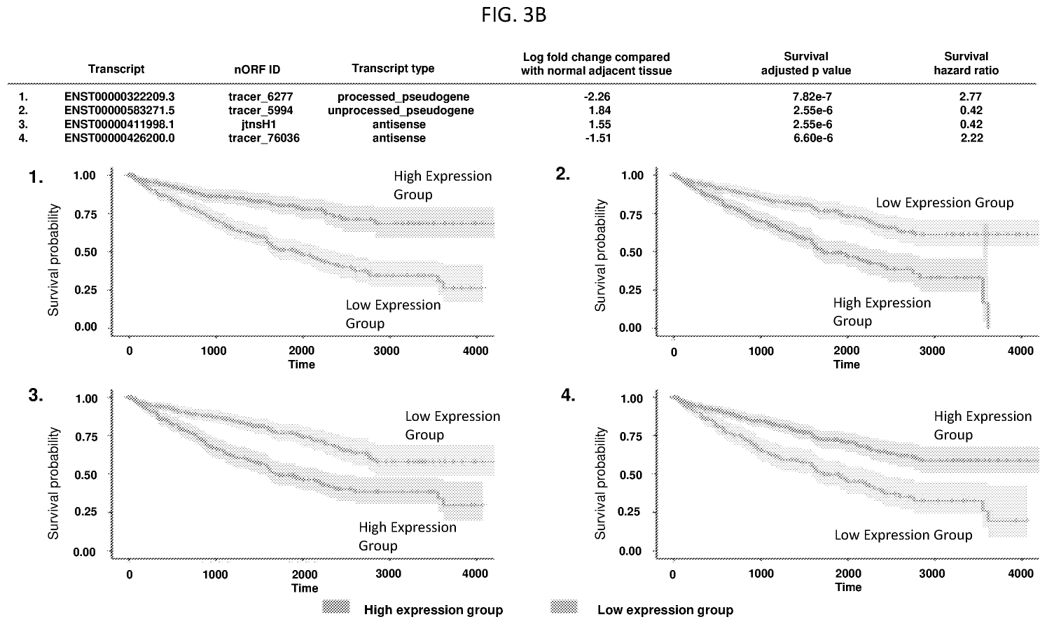

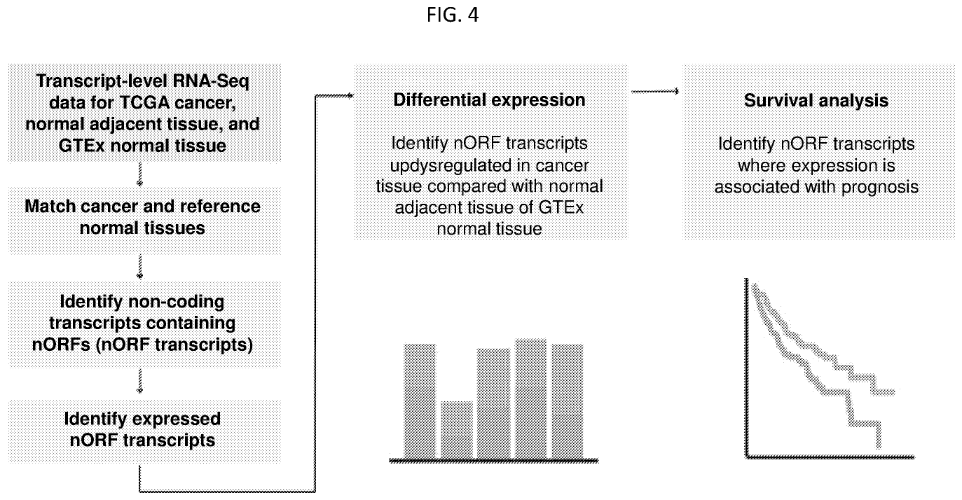

We have shown that nORF transcripts are frequently expressed across multiple cancer types and reference normal tissues, and that many of these nORF transcripts are transcriptionally dysregulated in cancers. To determine whether any differentially expressed nORF transcripts can be used as prognostic marker, we investigated the relationship between nORF transcript expression and overall patient survival, for nORF transcripts differentially expressed between cancers and NAT. We used survival data for TOGA cohorts provided by the UCSC Toil Recompute Compendium and divided each cohort into high and low expression groups for each nORF transcript, as detailed in Materials and Methods. We identified 43 nORF transcripts where expression was significantly associated with patient overall survival in at least one of the 12 cancer types included in this survival analysis, with an adjusted p value threshold of 0.05 (FIG. 3A). This suggested many nORF transcripts may have prognostic value, particularly in kidney clear cell carcinoma.

We investigated further nORF transcripts reproducibly differentially expressed both compared with NAT and GTEx normal tissue. For a subset of 33 nORF transcripts: (i) the transcript is reproducibly differentially expressed in cancer compared with NAT and GTEx normal tissue (ii) transcript expression is associated with prognosis (adjusted p<0.05) (iii) and transcripts up-regulated in cancer are associated with poor prognosis, and vice versa. Kaplan Meier survival curves are shown for the nORF transcripts most significantly associated with prognosis, in Kidney Clear Cell Carcinoma (FIG. 3B). We then embarked on a systematic investigation of predicting the structure and biological regulation of nORFs to infer their functions.

### Discussion

Through comprehensive analysis of RNA-Seq data from 22 cancer types, we have identified transcripts containing novel open reading frames and demonstrated that many nORF transcripts are frequently expressed in multiple cancers. Additionally, we have shown that many of these nORF transcripts are differentially expressed between cancer and normal tissue, and some of these nORF transcripts are uniquely differentially expressed in specific cancer types. Furthermore, we have shown that expression of some differentially expressed nORF transcripts have prognostic value—this is particularly convincing for four nORF transcripts reproducibly and uniquely identified as up-regulated in either liver hepatocellular carcinoma or lung adenocarcinoma, for which high expression was associated with poor prognosis.

### Materials and Methods

### TCGA and GTEx Transcriptome Processing

TOGA and GTEx RNA-Seq and survival data was downloaded from the TCGA TARGET GTEx′ cohort of the UCSC Toil Recompute Compendium. Transcriptome alignment had been performed using STAR (GRCh38) and transcript expression quantified using RSEM, using transcripts present in the GENCODE v23 genome annotation. Transcript-level RSEM expected counts, TOGA survival data and phenotype data were obtained. The GENCODE v23 and corresponding Ensembl v81 genome annotations were downloaded, and transcript and coding sequence properties were extracted from the annotation files using a custom script. RSEM expected counts provided by the UCSC Toil Recompute Compendium were log 2(expected_count+1) transformed, and this transformation was removed to produce raw expected counts for use in this analysis. All data processing was performed using R, R Studio, the R package Tidyverse and unix command line tools. The Ensembl genome annotation was processed in R using ensembl db, and genomic coordinates were processed using GenomicRanges. Set diagrams were produced using UpSetR.

### TCGA and GTEx Normal Sample Selection

Mappings of TOGA cancer tissue samples to normal adjacent tissue (NAT) and GTEx normal tissue were extracted from the phenotype data provided by the UCSC Toil Recompute Compendium. We included solid tumor TOGA cancer tissues with at least 50 samples, with matched NAT or GTEx normal tissue with at least 10 or 50 samples respectively—a less stringent threshold for inclusion was used for NAT because these samples were less abundant. RSEM expected count data was filtered to retain only selected samples and expressed transcripts prior to normalization and differential expression analysis. A single sample containing missing expected count values was excluded from this analysis.

### Identifying TCGA and GTEx Expressed Transcripts

Prior to library size normalization and differential expression analysis, transcripts with poor expression were excluded from analysis. Applying a CPM threshold to identify expressed transcripts prior to TMM normalization and differential expression analysis has been shown to improve false discovery rate and is recommended practice for edgeR. Expected counts were transformed to CPM and transcripts are classified as expressed if they had expected count greater than 0.5 CPM in at least 10% of the samples of a single cancer or normal tissue. Expressed transcripts are retained. Best practices for setting thresholds for transcript-level expression are poorly established, and the thresholds used in this study were, whilst informed by the literature, largely arbitrary.

### Selecting Matched Cancer and Normal Tissue Samples

To characterize the expression of transcripts encoding nORFs across multiple cancer types and corresponding normal tissues, we obtained transcript-level RNA-Seq expression data from the UCSC Toil Recompute Compendium. This dataset includes 11,194 cancer and normal adjacent tissue samples (NAT) from TCGA and 8,003 normal tissue samples from GTEx. We used metadata provided by the UCSC Toil Recompute Compendium to match cancer, NAT and GTEx normal tissues and determine the number of samples available for each tissue. To ensure consistent and reliable results, we included solid tumor TCGA cancer tissues with at least 50 samples, with matched NAT or GTEx normal tissue containing at least 10 or 50 samples respectively—a less stringent threshold for inclusion was used for NAT because these samples are less abundant. This resulted in a total of 7,885 samples across 22 cancer types from TCGA, together with 677 NAT samples and 4,010 GTEx normal samples.

NAT and GTEx normal tissues provide non-redundant reference tissues. NAT samples closely resemble cancer samples both as a result of reduced variation in patient differences and sample processing. However, NAT is affected by changes in the tumor microenvironment and samples are less abundant than GTEx normal tissue samples. Seven cancer tissues included in this study are matched to both NAT and GTEx normal tissue which allowed us to determine whether differential expression results are reproducible across different reference tissues.

### Identifying Transcripts Containing Novel Open Reading Frames

Genomic coordinates of nORFs with experimental evidence for translation were obtained from the nORFs.org database (norfs.org/home). Transcript genomic coordinates were obtained from the GENCODE v23 reference annotation. GffCompare was used to identify open reading frames and transcripts with completely matching intron chains. GffCompare performs stringent filtering to detect and remove redundant input transcripts, and this deduplication is described in detail in the documentation. Specifically, to achieve stringent deduplication of nORFs, GffCompare was run with nORF coordinates as the ‘reference set’ and transcript coordinates as the ‘query set’, with default parameters. The resultant ‘.refmap’ file containing information on overlaps between nORF and transcript coordinates was processed in R and annotated. nORF-transcript mappings identified by GffCompare were filtered to retain only those with a complete intron chain match, and for which the genomic coordinates of the nORF were completely contained within the transcript. nORFs present in multiple transcripts were excluded. Transcript biotypes were extracted from the GENCODE annotation file and open reading frames contained in protein-coding transcripts (transcripts with biotype: “protein_coding”, “IG_C_gene”, “IG_D_gene”, “IG_J_gene”, “IG_V_gene”, “TR_C_gene”, “TR_D_gene”, “TR_J_gene”, “TR_V_gene”) and rRNA transcripts were excluded. Novel and canonical ORF lengths were determined using ensembldb.

### RNA Sequencing Normalization

Normalization and differential expression were performed separately for comparison of cancer tissue with NAT and with GTEx normal tissue. RNA-Seq expected counts were normalized across samples using the trimmed mean of M-values (TMM) method to normalize for read depth and composition. As comparisons in differential expression were not made across transcripts, no normalization was introduced for effective transcript length.

### Identifying Frequently Expressed Transcripts

To identify frequently expressed transcripts, CPM values were calculated across all expressed transcripts following TMM normalization using edgeR. Transcripts were classed as frequently expressed if they had CPM greater than 0.5 in at least 70% of the samples in the normal or cancer tissue of interest.

### Transcript Differential Expression

Transcript differential expression was performed using all expressed transcripts to provide correct significance testing and improve reliability of dispersion estimation. The R package edgeR was used to perform differential expression analysis using a general linear model framework—this package was chosen as it is (i) highly cited (ii) suitable for transcript-level analysis (iii) compatible with non-integer expected counts from RSEM (iv) and exhibits fast performance on large datasets. A simple additive model with no intercept was constructed, with normal reference tissues and cancer tissues each represented by a single coefficient. The process used for differential expression analysis is detailed in the edgeR manual. Briefly, transcript-wise dispersions were estimated under the general linear model framework using the Cox-Reid profile-adjusted likelihood method, which takes into account multiple factors by fitting the described model. A negative binomial model was fitted for each transcript, and thresholded hypotheses were tested to provide meaningful p values and reliable control of false discovery rate. A fold change threshold of 1.5 or 2 was used to identify differentially expressed transcripts, with an adjusted p value threshold of 0.001. Coefficients representing cancer tissues and their corresponding normal reference tissues were compared under this framework. The Benjamini and Hochberg method was used to adjust p values for multiple testing and control false discovery rate.

### Patient Overall Survival Analysis

Overall survival (OS) analysis was performed using the R packages survival and survminer. nORF transcripts are included in survival analysis if they were differentially expressed in the cancer type of interest compared with NAT and were expressed at greater than 0.5 CPM in at least 70% of the samples in the cancer tissue cohort. For each cancer type and for the nORF transcript considered, the cohort was split into high and low expression groups. Groups were selected which were best segregated based on overall survival, using the Maximally Selected Rank Statistic, with at least 30% of patients assigned to each expression group to avoid forming groups with a small number of patients. Kaplan Meier curves were generated, and curves were compared using a log-rank test. The Benjamini and Hochberg method was used to adjust p values for multiple testing and control false discovery rate. A Cox proportional hazards regression model was fitted to overall survival data and hazard ratios were derived from the model coefficients. Both the Kaplan Meier and Cox proportional hazards regression models assume proportional hazards, where the hazard ratio between the high and low expression group remains constant over time.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a cancer in a subject comprising:
(a) identifying a sequence of a novel open reading frame (nORF) and a cancer associated therewith, wherein the sequence of the nORF is distinct from a canonical open reading frame (cORF) of a gene, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a noncancerous cell; and
(b) administering to the subject an inhibitor that reduces expression of the nORF to treat the cancer.

2. A method of treating a cancer in a subject comprising administering to the subject an inhibitor that reduces expression of a nORF; wherein the subject has previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a noncancerous cell.

3. The method of any one of claim 1 or 2, wherein the inhibitor comprises a small molecule, a polynucleotide, or a polypeptide.

4. The method of claim 3, wherein the polynucleotide comprises a miRNA, an antisense RNA, an shRNA, or an siRNA.

5. The method of claim 3, wherein the polypeptide comprises an antibody or antigen-binding fragment thereof.

6. The method of claim 5, wherein the antigen-binding fragment thereof is an scFv.

7. The method of any one of claims 3 to 6, wherein the inhibitor is encoded by a vector.

8. The method of claim 7, wherein the vector is a viral vector.

9. The method of claim 8, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

10. The method of claim 9, wherein the parvovirus viral vector is an adeno-associated virus (AAV) vector.

11. The method of claim 10, wherein the viral vector is a Retroviridae family viral vector.

12. The method of claim 11, wherein the Retroviridae family viral vector is a lentiviral vector.

13. The method of claim 11, wherein the Retroviridae family viral vector is an alpharetroviral vector or a gammaretroviral vector.

14. The method of any one of claims 10 to 13, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

15. The method of any one of claims 10 to 14, wherein the viral vector is a pseudotyped viral vector.

16. The method of claim 15, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

17. The method of claim 16, wherein the pseudotyped viral vector is a lentiviral vector.

18. The method of any one of claims 15 to 17, wherein the pseudotyped viral vector comprises one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

19. The method of claim 18, wherein the pseudotyped viral vector comprises a VSV-G envelope protein.

20. A method of treating a cancer in a subject comprising:
(a) identifying a sequence of a nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell; and
(b) administering to the subject an activator that increases expression of nORF to treat the cancer.

21. A method of treating a cancer in a subject comprising administering to the subject an activator that increases expression of a nORF; wherein the subject has previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell.

22. The method of claim 20 or 21, wherein the activator comprises a small molecule, a polynucleotide, or a polypeptide.

23. The method of claim 22, wherein the polynucleotide comprises an antisense RNA.

24. The method of claim 22, wherein the polypeptide comprises an antibody or antigen-binding fragment thereof.

25. The method of claim 24, wherein the antigen-binding fragment thereof is an scFv.

26. The method of any one of claims 20 to 25, wherein the activator is encoded by a vector.

27. The method of claim 26, wherein the vector is a viral vector.

28. A method of treating a cancer in a subject comprising:
(a) identifying a sequence of a nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell; and
(b) providing a protein encoded by the nORF to the subject treat the cancer.

29. A method of treating a cancer in a subject comprising providing a protein encoded by a nORF to the subject; wherein the subject has previously been identified with a sequence of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a noncancerous cell.

30. The method of claim 28 or 29, wherein the method comprises restoring the encoded protein product of the nORF.

31. The method of claim 30, wherein the therapy comprises providing the protein product or a polynucleotide encoding the protein product.

32. The method of claim 31, wherein the method comprises providing a vector comprising the polynucleotide encoding the protein product.

33. The method of claim 32, wherein the vector is a viral vector.

34. The method of claim 33, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

35. The method of claim 34, wherein the parvovirus viral vector is an AAV vector.

36. The method of claim 35, wherein the viral vector is a Retroviridae family viral vector.

37. The method of claim 36, wherein the Retroviridae family viral vector is a lentiviral vector.

38. The method of claim 36, wherein the Retroviridae family viral vector is an alpharetroviral vector or a gammaretroviral vector.

39. The method of any one of claims 34 to 37, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

40. The method of any one of claims 33 to 39, wherein the viral vector is a pseudotyped viral vector.

41. The method of claim 40, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

42. The method of claim 41, wherein the pseudotyped viral vector is a lentiviral vector.

43. The method of any one of claims 39 to 42, wherein the pseudotyped viral vector comprises one or more envelope proteins from a virus selected from vesicular stomatitis virus VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

44. The method of claim 43, wherein the pseudotyped viral vector comprises a VSV-G envelope protein.

45. The method of any one of claims 1 to 44, wherein the encoded protein product of the nORF is less than about 100 amino acids.

46. The method of any one of claims 1 to 45, further comprising performing a statistical analysis between the nORF and the cancer.

47. The method of claim 46, wherein the statistical analysis measures a positive or negative association between the nORF and the cancer.

48. The method of any one of claims 1 to 47, wherein the cancer is selected from the list consisting of breast invasive carcinoma, colon adenocarcinoma, esophageal carcinoma, head and neck squamous cell carcinoma, kidney chromophobe, kidney clear cell carcinoma, kidney papillary cell carcinoma, liver hepatocellular carcinoma, lung adenocarcinoma, lung squamous cell carcinoma, prostrate adenocarcinoma, stomach adenocarcinoma, thyroid carcinoma, and uterine corpus endometrioid carcinoma.

49. The method of any one of claims 1 to 48, wherein the nORF is selected from any one of Tables 1-5.
